SEQUENCE LISTING (13 sequences)

SEQ ID NO: 1 - protein, 408 aa, synthetic construct
FLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLSAIGGYIPEAPRDGQAYVRKDGEWVLLSTFL

SEQ ID NO: 2 - protein, 84 aa, synthetic construct
QNITEEFYQSTCSAVSKGYLSALRTGWYTSVITIELSNIKENKCNGTDAKVKLIKQELDKYKNAVTELQLLMQSTPACNSRARR

SEQ ID NO: 3 - protein, 408 aa, synthetic construct
FLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELLSAIGGYIPEAPRDGQAYVRKDGEWVLLSTFL

SEQ ID NO: 4 - protein, 84 aa, synthetic construct
QNITEEFYQSTCSAVSRGYFSALRTGWYTSVITIELSNIKETKCNGTDTKVKLIKQELDKYKNAVTELQLLMQNTPACNNRARR

SEQ ID NO: 5 - protein, 574 aa, Human respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 6 - protein, 574 aa, Human respiratory syncytial virus
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 7 - protein, 27 aa, synthetic construct
GYIPEAPRDGQAYVRKDGEWVLLSTFL

SEQ ID NO: 8 - protein, 126 aa, synthetic construct [sequence omitted]

SEQ ID NO: 9 - protein, 108 aa, synthetic construct
DIQMTQSPSSLSAAVGDRVTITCQASQDIVNYLNWYQQKPGKAPKLLIYVASNLETGVPSRFSGSGSGTDFSLTISSLQPEDVATYYCQQYDNLPLTFGGGTKVEIKR

SEQ ID NO: 10 - protein, 123 aa, synthetic construct
EVQLVESGGGVVQPGRSLRLSCAASGFSFSHYAMHWVRQAPGKGLEWVAVISYDGENTYYADSVKGRFSISRDNSKNTVSLQMNSLRPEDTALYYCARDRIVDDYYYYGMDVWGQGATVTVSS

SEQ ID NO: 11 - protein, 111 aa, synthetic construct
DIQMTQSPSSLSASVGDRVTITCQASQDIKKYLNWYHQKPGKVPELLMHDASNLETGVPSRFSGRGSGTDFTLTISSLQPEDIGTYYCQQYDNLPPLTFGGGTKVEIKRTV

SEQ ID NO: 12 - protein, 124 aa, synthetic construct
QVQLVQSGAEVKKPGATVKVSCKISGHTLIKLSIHWVRQAPGKGLEWMGGYEGEVDEIFYAQKFQHRLTVIADTATDTVYMELGRLTSDDTAVYFCGTLGVTVTEAGLGIDDYWGQGTLVTVSS

SEQ ID NO: 13 - protein, 108 aa, synthetic construct
EIVLTQSPGTLSLSPGERATLSCRASQIVSRNHLAWYQQKPGQAPRLLIFGASSRATGIPVRFSGSGSGTDFTLTINGLAPEDFAVYYCLSSDSSIFTFGPGTKVDFK

(19) United States
(12) Patent Application Publication
(10) Pub. No.: US 2026/0097113 A1
(43) Pub. Date: Apr. 9, 2026

(54) IMMUNOGENIC COMPOSITIONS COMPRISING AN RSV F PROTEIN TRIMER

(71) Applicant: Pfizer Inc., New York, NY (US)

(72) Inventors: Bryan Mark Balthazor, Defiance, MO (US); Miguel Angel Garcia, Midland Park, NJ (US); Sumit Goswami, Princeton, NJ (US); Lei Hu, New City, NY (US); Vamsi Krishna Mudhivarthi, King of Prussia, PA (US); Katherine Elizabeth Odneal, Ballwin, MO (US); Shuai Shi, Whippany, NJ (US); Serguei Tchessalov, Billerica, MA (US)

(21) Appl. No.: 19/115,478

(22) PCT Filed: Sep. 26, 2023
(86) PCT No.: PCT/IB2023/059541

§ 371 Date: Mar. 26, 2025

Related U.S. Application Data
(60) Provisional application No. 63/377,542, filed on Sep. 29, 2022.
(60) Provisional application No. 63/516,231, filed on Jul. 28, 2023.

(51) Int. Cl. A61K 39/155; A61K 9/08; A61K 9/19; A61K 39/00; A61K 47/02; A61K 47/18; A61K 47/26

(52) U.S. Cl. CPC A61K 39/155; A61K 9/08; A61K 9/19; A61K 47/02; A61K 47/183; A61K 47/26; A61K 2039/70; C12N 2760/18534

(57) ABSTRACT

The invention relates to aqueous and lyophilized immunogenic compositions comprising an RSV F protein trimer in the prefusion conformation. The invention further relates to methods for storing an aqueous immunogenic composition comprising an RSV F protein trimer in the prefusion conformation as well as to methods for lyophilizing such aqueous immunogenic compositions.

### REFERENCE TO SEQUENCE LISTING

This application is being filed electronically via Patent Center and includes an electronically submitted sequence listing in .xml format. The .xml file contains a sequence listing entitled “PC072868 Sequence Listing.xml” created on Aug. 12, 2022 and having a size of 15 KB. The sequence listing contained in this .xml file is part of the specification and is herein incorporated by reference in its entirety.

### FIELD OF THE INVENTION

The invention relates to aqueous and lyophilized immunogenic compositions comprising an RSV F protein trimer in the prefusion conformation. The invention further relates to methods for storing an aqueous immunogenic composition comprising an RSV F protein trimer in the prefusion conformation as well as to methods for lyophilizing such aqueous immunogenic compositions.

### BACKGROUND

Respiratory syncytial virus, or RSV, is a virus that infects the lungs and breathing passages. RSV is the leading cause of serious viral lower respiratory tract illness in infants worldwide and an important cause of respiratory illness in older adults. However, no vaccines have been approved for preventing RSV disease.

RSV is a member of the Pneumoviridae family. Its genome consists of a single-stranded, negative-sense RNA molecule that encodes 11 proteins, including nine structural proteins (three glycoproteins and six internal proteins) and two non-structural proteins. The structural proteins include three transmembrane surface glycoproteins: the attachment protein G, fusion protein F, and the small hydrophobic SH protein. There are two subtypes of RSV, A and B. They differ primarily in the G glycoprotein, while the sequence of the F glycoprotein is more conserved between the two subtypes.

The mature F glycoprotein has three general domains: ectodomain (ED), transmembrane domain TM, and a cytoplasmic tail (CT). The CT contains a single palmitoylated cysteine residue.

The F glycoprotein of human RSV is initially translated from the mRNA as a single 574-amino acid polypeptide precursor (referred to “F0” or “F0 precursor”), which contains a signal peptide sequence (amino acids 1-25) at the N-terminus. Upon translation the signal peptide is removed by a signal peptidase in the endoplasmic reticulum. The remaining portion of the F0 precursor (i.e., residues 26-574) may be further cleaved at two polybasic sites (a.a. 109/110 and 136/137) by cellular proteases (in particular furin), removing a 27-amino acid intervening sequence designated pep27 (amino acids 110-136) and generating two covalently-linked fragments designated F1 (C-terminal portion; amino acids 137-574) and F2 (N-terminal portion; amino acids 26-109). F1 contains a hydrophobic fusion peptide at its N-terminus and two heptad-repeat regions (HRA and HRB). HRA is near the fusion peptide, and HRB is near the TM domain. The F1 and F2 fragments are linked together through two disulfide bonds to form a F2-F1 heterodimer. Either the uncleaved F0 protein without the signal peptide sequence or a F1-F2 heterodimer can form a RSV F protomer. Three such protomers assemble to form the mature trimeric RSV F protein, which is a homotrimer of the three protomers.

The F proteins of subtypes A and B are about 90 percent identical in amino acid sequence. An example sequence of the F0 precursor polypeptide for the A subtype is provided in SEQ ID NO: 5 (A2 strain; GenBank GI: 138251; Swiss Prot P03420), and for the B subtype is provided in SEQ ID NO: 6 (18537 strain; GenBank GI: 138250; Swiss Prot P13843). SEQ ID NO: 5 and SEQ ID NO: 6 are both 574 amino acid sequences. The signal peptide sequence for SEQ ID NO: 5 and SEQ ID NO:6 has also been reported as amino acids 1-25 (GenBank and UniProt). In both sequences the TM domain is from approximately amino acids 530 to 550, but has alternatively been reported as 525-548. The cytoplasmic tail begins at either amino acid 548 or 550 and ends at amino acid 574, with the palmitoylated cysteine residue located at amino acid 550.

RSV F is a primary antigen explored for RSV vaccines. RSV F mediates fusion between the virion membrane and the host cellular membrane and also promotes the formation of syncytia. In the virion prior to fusion with the host cell membrane, the largest population of F molecules forms a lollipop-shaped structure, with the TM domain anchored in the viral envelope [Dormitzer, P. R., Grandi, G., Rappuoli, R., Nature Reviews Microbiol, 10, 807, 2012.; Mclellan J S, Ray W C, Peeples M E. Structure and function of respiratory syncytial virus surface glycoproteins. Curr Top Microbiol Immunol 2013; 372:83-104]. This conformation is referred to as the prefusion conformation. Prefusion RSV F is recognized by monoclonal antibodies (mAbs) D25, AM22, and MPE8, without discrimination between oligomeric states. AM14 is a mAb that binds a quaternary epitope and is specific to the trimeric form of RSV prefusion F [Gilman M S, Moin S M, Mas V et al. Characterization of a prefusion-specific antibody that recognizes a quaternary, cleavage-dependent epitope on the RSV fusion glycoprotein. PLOS Pathogens, 11 (7), 2015]. During RSV entry into cells, the F protein rearranges through an irreversible process from the metastable prefusion state (which may be referred to herein as “preF”), through an intermediate extended structure, to a highly stable postfusion state (“post-F”). During this rearrangement, the C-terminal coiled-coil of the prefusion molecule dissociates into its three constituent strands, which then wrap around the globular head and join three additional helices to form the postfusion six helix bundle. If a prefusion RSV F trimer is subjected to increasingly harsh chemical or physical conditions, such as elevated temperature, it undergoes structural changes. Initially, there is loss of trimeric structure (at least locally within the molecule), and then rearrangement to the postfusion form, and then denaturation of the domains.

To prevent viral entry, F-specific neutralizing antibodies presumably must bind the prefusion conformation of F on the virion, or potentially the extended intermediate, before the viral envelope fuses with a cellular membrane. Thus, the prefusion form of the F protein is considered the preferred conformation as the desired vaccine antigen [Ngwuta, J. O., Chen, M., Modjarrad, K., Joyce, M. G., Kanekiyo, M., Kumar, A., Yassine, H. M., Moin, S. M., Killikelly, A. M., Chuang, G. Y., Druz, A., Georgiev, I. S., Rundlet, E. J., Sastry, M., Stewart-Jones, G. B., Yang. Y., Zhang, B., Nason, M. C., Capella, C., Peeples, M., Ledgerwood, J. E., Mclellan, J. S., Kwong, P. D., Graham, B. S., Science Translat. Med., 14, 7, 309 (2015)]. Upon extraction from a membrane with surfactants such as Triton™ X-100, Triton™ X-114, NP-40, Brij™-35, Brij™-58, Tween™ 20, Tween™ 80, Octyl glucoside, Octyl thioglucoside, SDS, CHAPS, CHAPSO, or expression as an ectodomain, physical or chemical stress, or storage, the F glycoprotein readily converts to the postfusion form [Mclellan J S, Chen M, Leung S et al. Structure of RSV fusion glycoprotein trimer bound to a prefusion-specific neutralizing antibody. Science 340, 1113-1117 (2013); Chaiwatpongsakorn, S., Epand, R. F., Collins, P. L., Epand R. M., Peeples, M. E., J Virol. 85 (8): 3968-77 (2011); Yunus, A. S., Jackson T. P., Crisafi, K., Burimski, I., Kilgore, N. R., Zoumplis, D., Allaway, G. P., Wild, C. T., Salzwedel, K. Virology. 2010 Jan. 20; 396 (2): 226-37]. Therefore, the preparation of prefusion F as a vaccine antigen has remained a challenge. Since the neutralizing and protective antibodies function by interfering with virus entry, it is postulated that an F antigen that does not elicit prefusion specific antibodies is not expected to be as effective as an F antigen that elicits prefusion specific antibodies. Therefore, it is considered more desirable to utilize an F protein vaccine that contains a F protein immunogen in the prefusion form. Mutants of the RSV F protein have been provided to increase prefusion stability (see for example PCT application No WO2017/109629) and are promising vaccine candidates.

The RSV F protein is a particularly unstable protein due to its propensity to convert to the non-active and highly stable postfusion form. Constructs of the RSV F protein have been produced with the aim of stabilizing the RSV F protein as a trimer in the prefusion conformation. Such constructs include for example soluble forms of the RSV F protein comprising stabilizing mutations for example to create disulfide bonds within the F ectodomain as well as exogenous C-terminally fused trimerization domains. However, these constructs, although stabilized, may not be stable under more extreme conditions of temperature, pH or osmolality, for example, and under these conditions could be considered as unstable and prone to aggregation or loss of prefusion conformation.

Therefore, there is a need for aqueous immunogenic compositions comprising RSV F protein in the desired trimer and prefusion conformation and where the aggregation of RSV F protein and the loss of prefusion content is minimized. Such aqueous immunogenic compositions should be stable and suitable for use as a vaccine. Ideally, such composition can also be lyophilized and/or stored while minimizing the loss of prefusion content and/or aggregation of RSV F protein in the composition. It would also be advantageous to minimize the duration of the lyophilization process while maintaining acceptable levels of aggregation and prefusion content to enable large scale production of the immunogenic composition.

### SUMMARY OF THE INVENTION

The invention relates to an aqueous immunogenic composition comprising
(i) a first RSV F protein trimer in the prefusion conformation;
(ii) sodium chloride at a concentration of between about 20 mM and about 250 mM;
(iii) at least one of sucrose, mannitol and glycine at a concentration of between about 5 mg/mL and about 100 mg/mL; and
(iv) a buffer;
wherein the pH of said composition is between about 7 and about 8.

The invention also relates to a method for storing an aqueous immunogenic composition comprising an RSV F protein trimer in the prefusion conformation, comprising storing the composition at a temperature of at least about 15° C.

The invention further relates to a method for lyophilizing an aqueous immunogenic composition comprising an RSV F protein trimer in the prefusion conformation comprising the step of freezing the composition wherein said step comprises reducing the temperature to a freezing temperature comprised between −40° C. and −60° C. at a freezing ramp rate of at least about 0.3° C./min.

The invention further relates to a lyophilized immunogenic composition obtained or obtainable by any one of the lyophilization methods disclosed herein.

The invention further relates to kits comprising such lyophilized composition and a diluent for reconstituting the lyophilized composition.

### BRIEF DESCRIPTION OF DRAWINGS

FIG. 1 shows the percentage of High Molecular Weight Species (% HMMS) in various aqueous compositions comprising an RSV F protein trimer in the prefusion conformation at T0 or after 1 or 3 weeks storage at 25° C.

FIG. 2 shows the % HMMS in various aqueous compositions comprising an RSV F protein trimer in the prefusion conformation at T0 or after 1 or 3 weeks storage at 5° C.

FIG. 3 shows the % HMMS in various aqueous compositions comprising an RSV F protein trimer in the prefusion conformation at T0 or after 3 weeks storage at −20° C.

FIG. 4 shows the prefusion content in various aqueous compositions comprising an RSV F protein trimer in the prefusion conformation at T0 or after 1 or 3 weeks storage at 25° C.

FIG. 5 shows the prefusion content in various aqueous compositions comprising an RSV F protein trimer in the prefusion conformation at T0 or after 1 or 3 weeks storage at 5° C.

FIGS. 6A and 6B show the impact of pH on relative prefusion content for formulations at 4.5% sucrose, 50 mM NaCl held at 5° C. (FIG. 6A) and 25° C. (FIG. 6B) for 2 weeks.

FIGS. 6C and 6D show the impact of pH on % HMMS for formulations at 4.5% sucrose, 50 mM NaCl held at 5° C. (FIG. 6C) and 25° C. (FIG. 6D) for 2 weeks.

FIGS. 7A and 7B show the impact of sucrose level on relative prefusion content for formulations at pH 7, 50 mM NaCl held at 5° C. (FIG. 7A) and 25° C. (FIG. 7B) for 2 weeks.

FIGS. 7C and 7D show the impact of sucrose level on % HMMS for formulations at pH7, 50 mM NaCl held at 5° C. (FIG. 7C) and 25° C. (FIG. 7D) for 2 weeks.

FIGS. 8A and 8B show the impact of NaCl level on relative prefusion content for formulations at pH 7, with 4.5% sucrose held at 5° C. (FIG. 8A) and 25° C. (FIG. 8B) for 2 weeks.

FIGS. 8C and 8D show the impact of NaCl level on % HMMS for formulations at pH7, with 4.5% sucrose held at 5° C. (FIG. 8C) and 25° C. (FIG. 8D) for 2 weeks.

FIG. 9A discloses the percentage of prefusion content change as measured by AM14 ELISA and % HMMS change after lyophilization of various compositions comprising RSV F protein trimer of subtype A and B. The compositions shown in FIG. 9A as #1 to #10 correspond to the Formulation #1 to #10 of Table 12.

FIG. 9B discloses the percentage of prefusion content change as measured by AM22 Fab titration and % HMMS change after lyophilization of various compositions comprising RSV F protein trimer of subtype A and B. The compositions shown in FIG. 9A as #1 to #10 correspond to the Formulation #1 to #10 of Table 12.

### DETAILED DESCRIPTION OF THE INVENTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

### 1. Exemplary Embodiments (E) of the Invention

Exemplary embodiments (E) of the invention provided herein include:
E1. An aqueous immunogenic composition comprising
(i) a first RSV F protein trimer in the prefusion conformation;
(ii) sodium chloride at a concentration of between about 20 mM and about 250 mM;
(iii) at least one of sucrose, mannitol and glycine at a concentration of between about 5 mg/mL and about 100 mg/mL; and
(iv) a buffer;
wherein the pH of said composition is between about 7 and about 8.
E2. The aqueous immunogenic composition according to E1 wherein sodium chloride is at a concentration of about 20, 30, 40, 50, 60, 70, 80, 90, 100, 110, 120, 130, 140, 150, 160, 170, 180, 190, 200, 210, 220, 230, 240 or 250 mM.
E3. The aqueous immunogenic composition according to E1 wherein sodium chloride is at a concentration of between about 20 mM and about 100 mM.
E4. The aqueous immunogenic composition according to E1 wherein sodium chloride is at a concentration of between about 40 mM and about 60 mM.
E5. The aqueous immunogenic composition according to any one of E1 to E4 wherein sucrose is at a concentration of between about 10 mg/mL and about 100 mg/mL.
E6. The aqueous immunogenic composition according to any one of E1 to E4 wherein sucrose is at a concentration of about 5, 10, 20, 30, 40, 50, 60, 70, 80, 90 or 100 mg/mL.
E7. The aqueous immunogenic composition according to any one of E1 to E4 wherein sucrose is at a concentration of between about 10 mg/mL and about 90 mg/mL.
E8. The aqueous immunogenic composition according to any one of E1 to E4 wherein sucrose is at a concentration of between about 10 mg/mL and about 50 mg/mL.
E9. The aqueous immunogenic composition according to any one of E1 to E4 wherein sucrose is at a concentration of about 22.5 mg/mL.
E10. The aqueous immunogenic composition according to any one of E1 to E4 wherein sucrose is at a concentration of about 30 mg/mL.
E11. The aqueous immunogenic composition according to any one of E1 to E4 wherein sucrose is at a concentration of about 60 mg/mL
E12. The aqueous immunogenic composition according to any one of E1 to E4 wherein sucrose is at a concentration of about 90 mg/mL.
E13. The aqueous immunogenic composition according to any one of E1 to E4 wherein glycine is at a concentration of between about 10 mg/mL and about 100 mg/mL
E14. The aqueous immunogenic composition according to any one of E1 to E4 wherein glycine is at a concentration of about 5, 10, 20, 30, 40, 50, 60, 70, 80, 90 or 100 mg/mL.
E15. The aqueous immunogenic composition according to any one of E1 to E4 wherein glycine is at a concentration of between about 10 mg/mL and about 90 mg/mL.
E16. The aqueous immunogenic composition according to any one of E1 to E4 wherein glycine is at a concentration of between about 10 mg/mL and about 50 mg/mL.
E17. The aqueous immunogenic composition according to any one of E1 to E4 wherein glycine is at a concentration of about 22.5 mg/mL.
E18. The aqueous immunogenic composition according to any one of E1 to E4 wherein glycine is at a concentration of about 30 mg/mL.
E19. The aqueous immunogenic composition according to any one of E1 to E4 wherein glycine is at a concentration of about 60 mg/mL
E20. The aqueous immunogenic composition according to any one of E1 to E4 wherein glycine is at a concentration of about 90 mg/mL.
E21. The aqueous immunogenic composition according to any one of E1 to E4 wherein mannitol is at a concentration of between about 10 mg/mL and about 100 mg/mL.
E22. The aqueous immunogenic composition according to any one of E1 to E4 wherein mannitol is at a concentration of about 5, 10, 20, 30, 40, 50, 60, 70, 80, 90 or 100 mg/mL.
E23. The aqueous immunogenic composition according to any one of E1 to E4 wherein mannitol is at a concentration of between about 10 mg/mL and about 90 mg/mL.
E24. The aqueous immunogenic composition according to any one of E1 to E4 wherein mannitol is at a concentration of between about 10 mg/mL and about 50 mg/mL.
E25. The aqueous immunogenic composition according to any one of E1 to E4 wherein mannitol is at a concentration of about 22.5 mg/mL
E26. The aqueous immunogenic composition according to any one of E1 to E4 wherein mannitol is at a. concentration of about 30 mg/mL.
E27. The aqueous immunogenic composition according to any one of E1 to E4 wherein mannitol is at a concentration of about 60 mg/mL
E28. The aqueous immunogenic composition according to any one of E1 to E4 wherein mannitol is at a concentration of about 90 mg/mL.
E29. The aqueous immunogenic composition according to any one of E1 to E4 wherein sucrose is at a concentration of between about 10 mg/mL and about 70 mg/mL and mannitol is at a concentration of between about 10 mg/mL and about 70 mg/mL.
E30. The aqueous immunogenic composition according to E29 wherein the ratio of sucrose to mannitol is between 1 to 1 and 1 to 5, preferably between 1 to 2 and 1 to 4.
E31. The aqueous immunogenic composition according to E30 wherein the ratio of sucrose to mannitol is 1 to 2.
E32. The aqueous immunogenic composition according to any one of E1 to E4 wherein sucrose is at a concentration of about 30 mg/mL and mannitol is at a concentration of about 60 mg/mL.
E33. The aqueous immunogenic composition according to any one of E1 to E4 wherein sucrose is at a concentration of about 22.5 mg/mL and mannitol is at a concentration of about 45 mg/mL
E34. The aqueous immunogenic composition according to any one of E1 to E4 wherein sucrose is at a concentration of between about 10 mg/mL and about 70 mg/mL and glycine is at a concentration of between about 10 mg/mL and about 70 mg/mL.
E35. The aqueous immunogenic composition according to E34 wherein the ratio of sucrose to glycine is between 1 to 1 and 1 to 5, preferably 1 to 2 and 1 to 4.
E36. The aqueous immunogenic composition according to E35 wherein the ratio of sucrose to glycine is 1 to 2.
E37. The aqueous immunogenic composition according to any one of E1 to E4 wherein sucrose is at a concentration of about 30 mg/mL and glycine is at a concentration of about 60 mg/mL.
E38. The aqueous immunogenic composition according to any one of E1 to E4 wherein sucrose is at a concentration of about 22.5 mg/mL and glycine is at a concentration of about 45 mg/mL.
E39. The aqueous immunogenic composition according to any one of E1 to E4 wherein mannitol is at a concentration of between about 10 mg/mL and about 70 mg/mL and glycine is at a concentration of between about 10 mg/mL and 70 mg/mL.
E40. The aqueous immunogenic composition according to E39 wherein the ratio of mannitol to glycine is between 1 to 1 and 1 to 5, preferably between 1 to 2 and 1 to 4.
E41. The aqueous immunogenic composition according to E40 wherein the ratio of mannitol to glycine is 1 to 2.
E42. The aqueous immunogenic composition according to any one of E1 to E4 wherein mannitol is at a concentration of about 30 mg/mL and glycine is at a concentration of about 60 mg/mL
E43. The aqueous immunogenic composition according to any one of E1 to E4 wherein mannitol is at a concentration of about 22.5 mg/mL and glycine is at a concentration of about 45 mg/mL.
E44. The aqueous immunogenic composition according to any one of E1 to E43 wherein the composition further comprises a surfactant.
E45. The aqueous immunogenic composition according to E44 wherein the surfactant is selected from polysorbate 20 (Tween™20), polysorbate 40 (Tween™40), polysorbate 60 (Tween™60), polysorbate 65 (Tween™65), polysorbate 80 (Tween™80), polysorbate 85 (Tween™85), Triton™ N-101, Triton™ X-100, oxtoxynol 40, nonoxynol-9, triethanolamine, triethanolamine polypeptide oleate, polyoxyethylene-660 hydroxystearate (PEG-15, Solutol™ H15), polyoxyethylene-35-ricinoleate (Cremophor EL™), soy lecithin, poloxamer, hexadecylamine, octadecylamine, octadecyl amino acid esters, lysolecithin, dimethyl-dioctadecylammonium bromide, methoxyhexadecylgylcerol, pluronic polyols, polyamines (e.g., pyran, dextransulfate, poly IC, carbopol), peptides (e.g., muramyl peptide and dipeptide, dimethylglycine, tuftsin), oil emulsions, mineral gels (e.g., aluminum phosphate) and immune stimulating complexes (ISCOMS).
E46. The aqueous immunogenic composition according to E44 wherein the surfactant is a polysorbate.
E47. The aqueous immunogenic composition according to E46 wherein the surfactant is polysorbate 20 or polysorbate 80.
E48. The aqueous immunogenic composition according to E47 wherein the surfactant is polysorbate 80.
E49. The aqueous immunogenic composition according to any one of E44 to E48 wherein the concentration of the surfactant is from about 0.01 mg/ml to about 10 mg/ml, from about 0.01 mg/ml to about 5.0 mg/ml, from about 0.01 mg/ml to about 2.0 mg/ml, from about 0.01 mg/ml to about 1.0 mg/ml, from about 0.1 mg/ml to about 1.0 mg/ml, from about 0.1 mg/ml to about 0.5 mg/ml, from about 0.1 mg/ml to about 0.3 mg/ml or from about 0.1 mg/ml to about 0.25 mg/ml.
E50. The aqueous immunogenic composition according to any one of E44 to E48 wherein the concentration of the surfactant is about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29 or 0.30 mg/mL.
E51. The aqueous immunogenic composition according to any one of E44 to E48 wherein the concentration of the surfactant is about 0.15 mg/mL.
E52. The aqueous immunogenic composition according to any one of E44 to E48 wherein the concentration of the surfactant is about 0.20 mg/mL.
E53. The aqueous immunogenic composition according to any one of E1 to E52 wherein the buffer is selected from the group consisting of histidine, phosphate, phosphoric acid, ascorbate, maleic acid, glycine, ascorbic acid, bicarbonate and carbonic acid, gluconate, edetate, malate, imidazole, Tris, phosphate, and mixtures thereof. The buffer is preferably histidine or Tris.
E54. The aqueous immunogenic composition according E53 wherein the buffer is Tris (tris(hydroxymethyl)aminomethane).
E55. The aqueous immunogenic composition according to any one of E1 to E54 wherein the concentration of the buffer is between about 0.5 mM and about 50 mM, preferably about 5 mM to about 40 mM, more preferably about 10 mM to about 30 mM, increasingly preferably about 15 to about 25 mM. Preferably, the concentration of the buffer is about 15 mM, 16 mM, 17 mM, 18 mM, 19 mM, 20 mM, 21 mM, 22 mM, 23 mM, 24 mM, 25 mM, 26 mM, 27 mM, 28 mM, 29 mM or 30 mM.
E56. The aqueous immunogenic composition according to any one of E1 to E55 wherein the concentration of the buffer is about 15 mM.
E57. The aqueous immunogenic composition according to any one of E1 to E55 wherein the concentration of the buffer is about 20 mM.
E58. The aqueous immunogenic composition according to any one of E1 to E57 wherein the pH of the composition is about 7.0, 7.1, 7.2, 7.3, 7.4, 7.5, 7.6, 7.7, 7.8, 7.9 or 8.0 or is between about 7.1 and about 7.7, or is about 7.1, 7.2, 7.3, 7.4, 7.5, 7.6 or 7.7, more preferably 7.4.
E59. The aqueous immunogenic composition according to any one of E1 to E58 wherein the pH of the composition is about 7.4.
E60. The aqueous immunogenic composition according to E1, wherein the composition comprises
(i) a first RSV F protein trimer in the prefusion conformation;
(ii) sodium chloride at a concentration of between about 35 mM and about 65 mM;
(iii) sucrose, at a concentration of between about 20 mg/mL and about 40 mg/mL and mannitol at a concentration of between about 45 mg/mL and about 75 mg/mL;
(iv) a Tris buffer at a concentration of between about 15 mM and about 25 mM;
(v) polysorbate 80 at a concentration of between about 0.1 and about 0.3 mg/mL; wherein the pH of the composition is between 7.1 and 7.7.
E61. The aqueous immunogenic composition according to E1, wherein the composition comprises
(i) a first RSV F protein trimer in the prefusion conformation;
(ii) sodium chloride at a concentration of about 50 mM;
(iii) sucrose at a concentration of about 30 mg/mL and mannitol at a concentration of about 60 mg/mL;
(iv) a Tris buffer at a concentration of about 20 mM;
(v) polysorbate 80 at a concentration of about 0.2 mg/mL;
wherein the pH of the composition is about 7.4.
E62. The aqueous immunogenic composition according to E1, wherein the composition comprises
(i) a first RSV F protein trimer in the prefusion conformation;
(ii) sodium chloride at a concentration of about 37 mM;
(iii) sucrose at a concentration of about 22.5 mg/mL and mannitol at a concentration of about 45 mg/mL;
(iv) a Tris buffer at a concentration of about 15 mM;
(v) polysorbate 80 at a concentration of about 0.15 mg/mL;
wherein the pH of the composition is about 7.4.
E63. The aqueous immunogenic composition according to any one of E1 to E62 wherein the composition further comprises a preservative, preferably a preservative selected from the group consisting of benzethonium chloride, 2-phenoxyethanol, phenol and thimerosal, more preferably 2-phenoxyethanol. The concentration of preservative in the aqueous formulation is preferably at least 9 mg/mL, more preferably 10 mg/mL.
E64. The aqueous immunogenic composition according to any one of E1 to E63, wherein the first RSV F protein is a F protein of subtype A.
E65. The aqueous immunogenic composition according to any one of E1 to E63, wherein the first RSV F protein is a F protein of subtype B.
E66. The aqueous immunogenic composition according to E64 or E65, wherein the first RSV F protein is a mutant of wild type RSV F protein.
E67. The aqueous immunogenic composition according to E66, wherein the first RSV F protein displays introduced mutations in the amino acid sequence relative to the amino acid sequence of the corresponding wild-type RSV F protein and is immunogenic against the wild-type RSV F protein in the prefusion conformation or against a virus comprising the wild-type F protein. Said mutations can be for example amino acid substitutions, deletions, or additions relative to a wild-type RSV F protein.
E69. The aqueous immunogenic composition according to any one of E1 to E67 wherein the first RSV F protein is a mutant of a wild-type RSV F protein, wherein the introduced amino acid mutations are mutation of a pair of amino acid residues in a wild-type RSV F protein to a pair of cysteines. The introduced pair of cysteine residues allows for formation of a disulfide bond between the cysteine residues that stabilize the protein's conformation or oligomeric state, such as the prefusion conformation.
E70. The aqueous immunogenic composition according to E69, wherein the first RSV F protein comprises one of the following pairs of mutations: 55C and 188C: 155C and 290C; 103C and 148C; or 142C and 371C, such as S55C and L188C; S155C and S290C; A103C and I148C; or L142C and N371C.
E71. The aqueous immunogenic composition according to any one of E64 to E70, wherein the first RSV F protein comprises amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the prefusion conformation, but exposed to solvent in the postfusion conformation. Examples of the replacement amino acids include large aliphatic amino acids (Ile, Leu and Met) or large aromatic amino acids (His, Phe, Tyr and Trp).
E72. The aqueous immunogenic composition according to E71, wherein the first RSV F protein comprises a cavity filling mutation selected from the group consisting of:
   (1) substitution of S at positions 55, 62, 155, 190, or 290 with I, Y, L, H, or M;
   (2) substitution of T at position 54, 58, 189, 219, or 397 with I, Y, L, H, or M;
   (3) substitution of G at position 151 with A or H;
   (4) substitution of A at position 147 or 298 with I, L, H, or M;
   (5) substitution of V at position 164, 187, 192, 207, 220, 296, 300, or 495 with I, Y, H; and
   (6) substitution of R at position 106 with W.
E73. The aqueous immunogenic composition according to E72, wherein the first RSV F protein comprises at least one cavity filling mutation selected from the group consisting of: T54H, S190I, and V296I.
E74. The aqueous immunogenic composition according to any one of E64 to E73, wherein the first RSV F protein comprises electrostatic mutations, which decrease ionic repulsion or increase ionic attraction between residues in a protein that are proximate to each other in the folded structure.
E75. The aqueous immunogenic composition according to E74, wherein the first RSV F protein comprises an electrostatic substitution that reduces repulsive ionic interactions or increases attractive ionic interactions with acidic residues of Glu487 and Asp489 from another protomer of RSV F trimer.
E76. The aqueous immunogenic composition according to E74, wherein the first RSV F protein comprises an electrostatic mutation selected from the group consisting of:
   (1) substitution of E at position 82, 92, or 487 by D, F, Q, T, S, L, or H;
   (2) substitution of K at position 315, 394, or 399 by F, M, R, S, L, I, Q, or T;
   (3) substitution of D at position 392, 486, or 489 by H, S, N, T, or P; and
   (4) substitution of R at position 106 or 339 by F, Q, N, or W.
E77. The aqueous immunogenic composition according to any one of E64 to E76 wherein the first RSV F protein comprises a combination of two or more different types of mutations selected from engineered disulfide mutations, cavity filling mutations, and electrostatic mutations.
E78. The aqueous immunogenic composition according to any one of E64 to E77 wherein the first RSV F protein comprises a combination of mutations relative to the corresponding wild-type RSV F protein, wherein the combination of mutations is selected from the group consisting of:
   (1) combination of 103C, 148C, 190I, and 486S, preferably A103C, I148C, S190I, and D486S;
   (2) combination of 54H, 55C, 188C, and 486S, preferably T54H S55C L188C D486S;
   (3) combination of 54H, 103C, 148C, 190I, 296I, and 486S, preferably T54H, A103C, I148C, S190I, V296I, and D486S;
   (4) combination of 54H, 55C, 142C, 188C, 296I, and 371C, preferably T54H, S55C, L142C, L188C, V296I, and N371C;
   (5) combination of 55C, 188C, and 486S, preferably S55C, L188C, and D486S;
   (6) combination of 54H, 55C, 188C, and 190I, preferably T54H, S55C, L188C, and S190I;
   (7) combination of 55C, 188C, 190I, and 486S, preferably S55C, L188C, S190I, and D486S;
   (8) combination of 54H, 55C, 188C, 190I, and 486S, preferably T54H, S55C, L188C, S190I, and D486S;
   (9) combination of 155C, 190I, 290C, and 486S, preferably S155C, S190I, S290C, and D486S;
   (10) combination of 54H, 55C, 142C, 188C, 296I, 371C, 486S, 487Q, and 489S, preferably T54H, S55C, L142C, L188C, V296I, N371C, D486S, E487Q, and D489S;
   (11) combination of 54H, 155C, 190I, 290C, and 296I, preferably T54H, S155C, S190I, S290C, and V296I, and
   (12) combination of 155C, 190F, 290C, and 207L, preferably S155C, S190F, S290C, and V207L.
E79. The aqueous immunogenic composition according to any one of E64 to E67 wherein the first RSV F protein comprise a combination of mutations relative to the corresponding wild-type RSV F protein, wherein the combination of mutations is selected from the group consisting of:
   (1) combination of 215P and 486N, preferably S215P and D486N,
   (2) combination of 66E, 215P, and 486N, preferably K66E, S215P, and D486N,
   (3) combination of 66E, 76V, 215P, and 486N, preferably K66E, 176V, S215P, and D486N, and,
   (4) combination of 66E, 671, 76V, 215P, and 486N, preferably K66E, N671, 176V, S215P, and D486N.
E80. The aqueous immunogenic composition according to any one of E64 to E78 wherein the first RSV F protein comprises the mutations 103C, 148C, 190I, and 486S, preferably A103C, I148C, S190I, and D486S.
E81. The aqueous immunogenic composition according to any one of E64 to E80 wherein the first RSV F protein comprises a trimerization domain. The RSV F protein comprises two covalently-linked fragments designated F1 (C-terminal portion; amino acids 137-574) and F2 (N-terminal portion; amino acids 26-109. The F1 and F2 fragments are linked together through two disulfide bonds to form a F2-F1 heterodimer. Either the uncleaved F0 protein without the signal peptide sequence or a F1-F2 heterodimer can form a RSV F protomer. Three such protomers assemble to form the mature trimeric RSV F protein, which is a homotrimer of the three protomers. The trimerization domain promotes the formation of a trimer of three F2-F1 heterodimers. Several exogenous multimerization domains that promote formation of stable trimers of soluble proteins are known in the art. The trimerization domain is preferably selected from the group consisting of:
(1) the GCN4 leucine zipper (Harbury et al. 1993 Science 262:1401-1407); (2) the trimerization motif from the lung surfactant protein (Hoppe et al. 1994 February S Lett 344:191-195); (3) collagen (McAlinden et al. 2003 Biol Chem 278:42200-42207); and (4) the phage T4 fibritin foldon (Miroshnikov et al. 1998 Protein Eng 11:329-414).
E82. The aqueous immunogenic composition according to E81 wherein the trimerization domain is the phage T4 fibritin foldon of SEQ ID NO: 7.
E83. The aqueous immunogenic composition according to E81 or E82 wherein the trimerization domain is linked to the first RSV protein at the C-terminus of F1 polypeptide.
E84. The aqueous immunogenic composition according to E83 wherein the trimerization domain is linked to the first RSV protein at the C-terminus of F1 polypeptide via a linker.
E85. The aqueous immunogenic composition according to E84 wherein the linker is selected from GG, GS, or SAIG.
E86. The aqueous immunogenic composition according to E81 wherein the trimerization domain is a T4 foldon fibritin domain and is linked to the first RSV protein at the C-terminus of F1 polypeptide via a SAIG linker.
E87. The aqueous immunogenic composition according to any one of E64 to E86 wherein the first RSV F protein comprises two separate polypeptide chains, said separate polypeptide chains being the F1 polypeptide and the F2 polypeptide.
E88. The aqueous immunogenic composition according to E87 wherein the F2 polypeptide is linked to the F1 polypeptide by one, two, three, four or five disulfide bonds to form a F2-F1 heterodimer.
E89. The aqueous immunogenic composition according to any one of E64 to E86 wherein the first RSV F protein is in the form a single chain protein, wherein the F2 polypeptide is linked to the F1 polypeptide by a peptide bond or a linker.
E90. The aqueous immunogenic composition according to E89 wherein the linker is selected from G, GG. GGG, GS, or SAIG.
E91. The aqueous immunogenic composition according to E89 wherein the linker is full length pep27 sequence or a fragment thereof.
E92. The composition according to any one of E87 to E91 wherein the F1 polypeptide of the first RSV F protein lacks the entire cytoplasmic domain or the entire cytoplasmic domain and a portion of or the entire transmembrane domain.
E93. The aqueous immunogenic composition according to E87 to E92 wherein amino acid residues 514 through 574 are absent from the F1 polypeptide of the first RSV F protein.
E94. The aqueous immunogenic composition according to E87 to E93 wherein the F2 polypeptide chain has the same length as the full-length F2 polypeptide of the corresponding wild-type RSV F protein or has deletions, such as deletions of 1, 2, 3, 4, 5, 6, 7, or 8 amino acid residues from the N-terminus or C-terminus of the F2 polypeptide.
E95. The aqueous immunogenic composition according to any one of E1 to E65 wherein the first RSV F protein is an RSV protein mutant as disclosed in WO2017/109629 which is hereby incorporated by reference in its entirety.
E96. The aqueous immunogenic composition according to any one of E1 to E65 wherein the first RSV F protein is an RSV protein mutant as disclosed in WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2022/002894 and WO2018/109220 which are all hereby incorporated by reference in their entirety.
E97. The aqueous immunogenic composition according to any one of E1 to E96 wherein the composition further comprises a second RSV F protein trimer in the prefusion conformation.
E98. The aqueous immunogenic composition according to E97, wherein the second RSV F protein is a F protein of subtype A.
E99. The aqueous immunogenic composition according to E97, wherein the second RSV F protein is a F protein of subtype B.
E100. The aqueous immunogenic composition according to any one of E97 to E99, wherein the second RSV F protein is a mutant of wild type RSV F protein.
E101. The aqueous immunogenic composition according to E100, wherein the second RSV F protein displays introduced mutations in the amino acid sequence relative to the amino acid sequence of the corresponding wild-type RSV F protein and is immunogenic against the wild-type RSV F protein in the prefusion conformation or against a virus comprising the wild-type F protein. Said mutations can be, for example, amino acid substitutions, deletions, or additions relative to a wild-type RSV F protein.
E102. The aqueous immunogenic composition according to any one of E97 to E101 wherein the second RSV F protein is a mutant of a wild-type RSV F protein, wherein the introduced amino acid mutations are mutation of a pair of amino acid residues in a wild-type RSV F protein to a pair of cysteines. The introduced pair of cysteine residues allows for formation of a disulfide bond between the cysteine residues that stabilize the protein's conformation or oligomeric state, such as the prefusion conformation.
E103. The aqueous immunogenic composition according to E102, wherein the second RSV F protein comprises one of the following pairs of mutations: 55C and 188C; 155C and 290C; 103C and 148C; or 142C and 371C, such as S55C and L188C; S155C and S290C; A103C and I148C; or L142C and N371C.
E104. The aqueous immunogenic composition according to any one of E97 to E103, wherein the second RSV F protein comprises amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the prefusion conformation but exposed to solvent in the postfusion conformation. Examples of the replacement amino acids include large aliphatic amino acids (Ile, Leu and Met) or large aromatic amino acids (His, Phe, Tyr and Trp).
E105. The aqueous immunogenic composition according to E104, wherein the second RSV F protein comprises comprise a cavity filling mutation selected from the group consisting of:
   (1) substitution of S at positions 55, 62, 155, 190, or 290 with I, Y, L, H, or M;
   (2) substitution of T at position 54, 58, 189, 219, or 397 with I, Y, L, H, or M;
   (3) substitution of G at position 151 with A or H;
   (4) substitution of A at position 147 or 298 with I, L, H, or M;
   (5) substitution of V at position 164, 187, 192, 207, 220, 296, 300, or 495 with I, Y, H; and
   (6) substitution of R at position 106 with W.
E106. The aqueous immunogenic composition according to E105, wherein the second RSV F protein comprises at least one cavity filling mutation selected from the group consisting of: T54H, S190I, and V296I.
E107. The aqueous immunogenic composition according to any one of E97 to E106, wherein the second RSV F protein comprises electrostatic mutations, which decrease ionic repulsion or increase ionic attraction between residues in a protein that are proximate to each other in the folded structure.
E108. The aqueous immunogenic composition according to E107, wherein the second RSV F protein comprises an electrostatic substitution that reduces repulsive ionic interactions or increases attractive ionic interactions with acidic residues of Glu487 and Asp489 from another protomer of RSV F trimer.
E109. The aqueous immunogenic composition according to E107, wherein the second RSV F protein comprises an electrostatic mutation selected from the group consisting of:
   (1) substitution of E at position 82, 92, or 487 by D, F, Q, T, S, L, or H;
   (2) substitution of K at position 315, 394, or 399 by F, M, R, S, L, I, Q, or T;
   (3) substitution of D at position 392, 486, or 489 by H, S, N, T, or P; and
   (4) substitution of R at position 106 or 339 by F, Q, N, or W.
E110. The aqueous immunogenic composition according to any one of E97 to E109 wherein the second RSV F protein comprises a combination of two or more different types of mutations selected from engineered disulfide mutations, cavity filling mutations, and electrostatic mutations.
E111. The aqueous immunogenic composition according to any one of E97 to E110 wherein the second RSV F protein comprise a combination of mutations relative to the corresponding wild-type RSV F protein, wherein the combination of mutations is selected from the group consisting of:
   (1) combination of 103C, 148C, 190I, and 486S, preferably A103C, I148C, S190I, and D486S;
   (2) combination of 54H, 55C, 188C, 486S, preferably T54H S55C L188C D486S;
   (3) combination of 54H, 103C, 148C, 190I, 296I, and 486S, preferably T54H, A103C, I148C, S190I, V296I, and D486S;
   (4) combination of 54H, 55C, 142C, 188C, 296I, and 371C, preferably T54H, S55C, L142C, L188C, V296I, and N371C;
   (5) combination of 55C, 188C, and 486S, preferably S55C, L188C, and D486S;
   (6) combination of 54H, 55C, 188C, and 190I, preferably T54H, S55C, L188C, and S190I;
   (7) combination of 55C, 188C, 190I, and 486S, preferably S55C, L188C, S190I, and D486S;
   (8) combination of 54H, 55C, 188C, 190I, and 486S, preferably T54H, S55C, L188C,
   S190I, and D486S;
   (9) combination of 155C, 190I, 290C, and 486S, preferably S155C, S190I, S290C, and D486S;
   (10) combination of 54H, 55C, 142C, 188C, 296I, 371C, 486S, 487Q, and 489S, preferably T54H, S55C, L142C, L188C, V296I, N371C, D486S, E487Q, and D489S;
   (11) combination of 54H, 155C, 190I, 290C, and 296I, preferably T54H, S155C, S190I, S290C, and V296I, and
   (12) combination of 155C, 190F, 290C, and 207L, preferably S155C, S190F, S290C, and V207L.
E112. The aqueous immunogenic composition according to any one of E97 to E110 wherein the second RSV F protein comprise a combination of mutations relative to the corresponding wild-type RSV F protein, wherein the combination of mutations is selected from the group consisting of:
   (1) combination of 215P and 486N, preferably S215P and D486N,
   (2) combination of 66E, 215P, and 486N, preferably K66E, S215P, and D486N,
   (3) combination of 66E, 76V, 215P, and 486N, preferably K66E, 176V, S215P, and D486N, and,
   (4) combination of 66E, 671, 76V, 215P, and 486N, preferably K66E, N671, 176V, S215P, and D486N.
E113. The aqueous immunogenic composition according to any one of E97 to E111 wherein the second RSV F protein comprises the mutations 103C, 148C, 190I, and 486S, preferably A103C, I148C, S190I, and D486S.
E114. The aqueous immunogenic composition according to any one of E97 to E113 wherein the second RSV F protein comprises a trimerization domain. The trimerization domain promotes the formation of trimer of three F2-F1 heterodimers. Several exogenous multimerization domains that promote formation of stable trimers of soluble proteins are known in the art. The trimerization domain is preferably selected from the group consisting of:
(1) the GCN4 leucine zipper (Harbury et al. 1993 Science 262:1401-1407); (2) the trimerization motif from the lung surfactant protein (Hoppe et al. 1994 February S Lett 344:191-195); (3) collagen (McAlinden et al. 2003 Biol Chem 278:42200-42207); and (4) the phage T4 fibritin foldon (Miroshnikov et al. 1998 Protein Eng 11:329-414).
E115. The aqueous immunogenic composition according to E114 wherein the trimerization domain is the phage T4 fibritin foldon of SEQ ID NO: 7.
E116. The aqueous immunogenic composition according to E114 or E115 wherein the trimerization domain is linked to the second RSV protein at the C-terminus of F1 polypeptide.
E117. The aqueous immunogenic composition according to E116 wherein the trimerization domain is linked to the second RSV protein at the C-terminus of F1 polypeptide via a linker.
E118. The aqueous immunogenic composition according to E117 wherein the linker is selected from GG, GS, or SAIG.
E119. The aqueous immunogenic composition according to E114 wherein the trimerization domain is a T4 foldon fibritin domain and is linked to the second RSV protein at the C-terminus of F1 polypeptide via a SAIG linker.
E120. The aqueous immunogenic composition according to any one of E97 to E119 the second RSV F protein comprises two separate polypeptide chains, said separate polypeptide chains being the F1 polypeptide and the F2 polypeptide.
E121. The aqueous immunogenic composition according to E120 wherein the F2 polypeptide is linked to the F1 polypeptide by one, two, three, four or five disulfide bonds to form a F2-F1 heterodimer.
E122. The aqueous immunogenic composition according to any one of E97 to E119 wherein the second RSV F protein is in the form a single chain protein, wherein the F2 polypeptide is linked to the F1 polypeptide by a peptide bond or a linker.
E123. The aqueous immunogenic composition according to E122 wherein the linker is selected from G, GG, GGG, GS, or SAIG.
E124. The aqueous immunogenic composition according to E122 wherein the linker is full length pep27 sequence or a fragment thereof.
E125. The composition according to any one of E120 to E124 wherein the F1 polypeptide of the second RSV F protein lacks the entire cytoplasmic domain or the entire cytoplasmic domain and a portion of or all entire transmembrane domain.
E126. The aqueous immunogenic composition according to E120 to E125 wherein amino acid residues 514 through 574 are absent from the F1 polypeptide of the second RSV F protein.
E127. The aqueous immunogenic composition according to E120 to E126 wherein the F2 polypeptide chain has the same length as the full-length F2 polypeptide of the corresponding wild-type RSV F protein or has deletions, such as deletions of 1, 2, 3, 4, 5, 6, 7, or 8 amino acid residues from the N-terminus or C-terminus of the F2 polypeptide.
E128. The aqueous immunogenic composition according to E97 wherein the second RSV F protein is an RSV protein mutant as disclosed in WO2017/109629 which is hereby incorporated by reference in its entirety.
E129. The aqueous immunogenic composition according to any one of E97 wherein the second RSV F protein is an RSV protein mutant as disclosed in WO2009/079796, WO2010/149745, WO2011/008974, WO2014/160463, WO2014/174018, WO2014/202570, WO2015/013551, WO2015/177312, WO2017/005848, WO2017/174564, WO2017/005844 and WO2018/109220 which are all hereby incorporated by reference in their entirety.
E130. The aqueous immunogenic composition according to E99 to E129 wherein the first RSV F protein is of subtype A and the second RSV F protein is of subtype B.
E131. The aqueous immunogenic composition according to E130 wherein the first and second RSV F protein comprise the mutations A103C, I148C, S190I, and D486S.
E132. The aqueous immunogenic composition according to E130 and E131 wherein the first and the second RSV F protein comprise
(a) a F1 polypeptide lacking amino acid residues 514 to 574, and,
(b) a F2 polypeptide linked to the F1 polypeptide at least by one disulfide bond, and, (c) a T4 foldon fibritin domain linked to at the C-terminus of F1 polypeptide via a SAIG linker.
E133. The aqueous immunogenic composition according to any one of E1 to E63 and E97 to E129 wherein the first RSV F protein comprises an F1 polypeptide of SEQ ID NO: 1 and an F2 polypeptide of SEQ ID NO: 2.
E134. The aqueous immunogenic composition according to any one of E97 and E130 to 133 wherein the second RSV F protein comprises an F1 polypeptide of SEQ ID NO:3 and an F2 polypeptide of SEQ ID NO: 4
E135. The aqueous immunogenic composition according to any one of E1 to E134 wherein the concentration of the first RSV F protein is about 0.01 mg/mL to about 10 mg/mL, preferably about 0.01 mg/mL to about 5 mg/mL, more preferably about 0.01 mg/mL to about 1 mg/mL.
E136. The aqueous immunogenic composition according to E135 wherein the concentration of the first RSV F protein is about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.88, 0.85, 0.86, 0.87, 0.88, 0.89, 0.90, 0.91, 0.92, 0.93, 0.94, 0.95, 0.96, 0.97, 0.98, 0.99 or 1 mg/mL.
E137. The aqueous immunogenic composition according to E136 wherein the concentration of the first RSV F protein is about 0.08 mg/mL.
E138. The aqueous immunogenic composition according to E136 wherein the concentration of the first RSV F protein is about 0.12 mg/mL.
E139. The aqueous immunogenic composition according to any one of E97 to E138 wherein the concentration of the second RSV F protein is about 0.01 mg/mL to about 10 mg/mL, preferably about 0.01 mg/mL to about 5 mg/mL, more preferably about 0.01 mg/mL to about 1 mg/mL.
E140. The aqueous immunogenic composition according to E138 wherein the concentration of the second RSV F protein is about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.88, 0.85, 0.86, 0.87, 0.88, 0.89, 0.90, 0.91, 0.92, 0.93, 0.94, 0.95, 0.96, 0.97, 0.98, 0.99 or 1 mg/mL.
E141. The aqueous immunogenic composition according to E138 wherein the concentration of the second RSV F protein is about 0.08 mg/mL.
E142. The aqueous immunogenic composition according to E138 wherein the concentration of the second RSV F protein is about 0.12 mg/mL.
E143. The aqueous immunogenic composition according to any one of E1 to E142 wherein the aqueous immunogenic composition further comprises an adjuvant.
E144. The aqueous immunogenic composition according to E143 wherein the adjuvant is selected from aluminum salts (alum), such as aluminum hydroxide, aluminum phosphate or aluminum sulfate, saponins, such as Stimulon™ QS-21 (Antigenics, Framingham, MA.), combination of 3-O-desacyl-4′-monophosphoryl lipid A (MPL™) and QS-21 in liposomes such as AS01 or ALFQ, or synthetic polynucleotides such as oligonucleotides containing a CpG motif. In a preferred embodiment, the adjuvant is aluminum hydroxide. In another preferred embodiment, the adjuvant is an oligonucleotide containing a CpG motif. In another preferred embodiment, the adjuvant comprises aluminum hydroxide and an oligonucleotide containing a CpG motif. In another preferred embodiment, the adjuvant is AS01 or ALFQ.
E145. The aqueous immunogenic composition according to any one of E1 to E144 wherein the percentage of HMMS in the composition is less than about 25, 24, 23, 22, 21, 20, 19, 18, 17, 16, 15, 14, 13, 12, 11 or 10% after 1 week at 25° C.
E146. The aqueous immunogenic composition according to any one of E1 to E144 wherein the percentage of HMMS in the composition is less than about 20% after 1 week at 25° C.
E147. The aqueous immunogenic composition according to any one of E1 to E144 wherein the percentage of HMMS in the composition is less than about 15% after 1 week at 25° C.
E148. The aqueous immunogenic composition according to any one of E1 to E144 wherein the percentage of HMMS in the composition is less than about 10% after 1 week at 25° C.
E149. The aqueous immunogenic composition according to any one of E1 to E148 wherein the percentage of HMMS in the composition is less than about 25, 24, 23, 22, 21, 20, 19, 18, 17, 16, 15, 14, 13, 12, 11 or 10% after 2 weeks at 25° C.
E150. The aqueous immunogenic composition according to any one of E1 to E148 wherein the percentage of HMMS in the composition is less than about 20% after 2 weeks at 25° C.
E151. The aqueous immunogenic composition according to any one of E1 to E148 wherein the percentage of HMMS in the composition is less than about 15% after 2 weeks at 25° C.
E152. The aqueous immunogenic composition according to any one of E1 to E148 wherein the percentage of HMMS in the composition is less than about 10% after 2 weeks at 25° C.
E153. The aqueous immunogenic composition according to any one of E1 to E152 wherein the percentage of HMMS in the composition is less than about 25, 24, 23, 22, 21, 20, 19, 18, 17, 16, 15, 14, 13, 12, 11 or 10% after 3 weeks at 25° C.
E154. The aqueous immunogenic composition according to any one of E1 to E152 wherein the percentage of HMMS in the composition is less than about 20% after 3 weeks at 25° C.
E155. The aqueous immunogenic composition according to any one of E1 to E152 wherein the percentage of HMMS in the composition is less than about 15% after 3 weeks at 25° C.
E156. The aqueous immunogenic composition according to any one of E1 to E152 wherein the percentage of HMMS in the composition is less than about 10% after 3 weeks at 25° C.
E157. The aqueous immunogenic composition according to any one of E145 to E156 wherein the percentage of HMMS in the composition is measured by size exclusion chromatography (SEC-HPLC), preferably as disclosed in Example 1.
E158. The aqueous immunogenic composition according to any one of E1 to E157 wherein the percentage of RSV F protein in the prefusion conformation in the composition is higher than about 60% after 1 week at 25° C.
E159. The aqueous immunogenic composition according to any one of E1 to E157 wherein the percentage of RSV F protein in the prefusion conformation in the composition is higher than about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% after 1 week at 25° C.
E160. The aqueous immunogenic composition according to any one of E1 to E157 wherein the percentage of RSV F protein in the prefusion conformation in the composition is higher than about 65% after 1 week at 25° C.
E161. The aqueous immunogenic composition according to any one of E1 to E157 wherein the percentage of RSV F protein in the prefusion conformation in the composition is higher than about 70% after 1 week at 25° C.
E162. The aqueous immunogenic composition according to any one of E1 to E157 wherein the percentage of RSV F protein in the prefusion conformation in the composition is higher than about 75% after 1 week at 25° C.
E163. The aqueous immunogenic composition according to any one of E1 to E157 wherein the percentage of RSV F protein in the prefusion conformation in the composition is higher than about 80% after 1 week at 25° C.
E164. The aqueous immunogenic composition according to any one of E1 to E157 wherein the percentage of RSV F protein in the prefusion conformation in the composition is higher than about 85% after 1 week at 25° C.
E165. The aqueous immunogenic composition according to any one of E1 to E157 wherein the percentage of RSV F protein in the prefusion conformation in the composition is higher than about 90% after 1 week at 25° C.
E166. The aqueous immunogenic composition according to any one of E1 to E165 wherein the percentage of RSV F protein in the prefusion conformation in the composition is higher than about 60% after 2 weeks at 25° C.
E167. The aqueous immunogenic composition according to any one of E1 to E165 wherein the percentage of RSV F protein in the prefusion conformation in the composition is higher than about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% after 2 weeks at 25° C.
E168. The aqueous immunogenic composition according to any one of E1 to E165 wherein the percentage of RSV F protein in the prefusion conformation in the composition is higher than about 65% after 2 weeks at 25° C.
E169. The aqueous immunogenic composition according to any one of E1 to E165 wherein the percentage of RSV F protein in the prefusion conformation in the composition is higher than about 70% after 2 weeks at 25° C.
E170. The aqueous immunogenic composition according to any one of E1 to E165 wherein the percentage of RSV F protein in the prefusion conformation in the composition is higher than about 75% after 2 weeks at 25° C.
E171. The aqueous immunogenic composition according to any one of E1 to E165 wherein the percentage of RSV F protein in the prefusion conformation in the composition is higher than about 80% after 2 weeks at 25° C.
E172. The aqueous immunogenic composition according to any one of E1 to E165 wherein the percentage of RSV F protein in the prefusion conformation in the composition is higher than about 85% after 2 weeks at 25° C.
E173. The aqueous immunogenic composition according to any one of E1 to E165 wherein the percentage of RSV F protein in the prefusion conformation in the composition is higher than about 90% after 2 weeks at 25° C.
E174. The aqueous immunogenic composition according to any one of E1 to E165 wherein the percentage of RSV F protein in the prefusion conformation in the composition is higher than about 60% after 3 weeks at 25° C.
E175. The aqueous immunogenic composition according to any one of E1 to E174 wherein the percentage of RSV F protein in the prefusion conformation in the composition is higher than about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% after 3 weeks at 25° C.
E176. The aqueous immunogenic composition according to any one of E1 to E174 wherein the percentage of RSV F protein in the prefusion conformation in the composition is higher than about 65% after 3 weeks at 25° C.
E177. The aqueous immunogenic composition according to any one of E1 to E174 wherein the percentage of RSV F protein in the prefusion conformation in the composition is higher than about 70% after 3 weeks at 25° C.
E178. The aqueous immunogenic composition according to any one of E1 to E174 wherein the percentage of RSV F protein in the prefusion conformation in the composition is higher than about 75% after 3 weeks at 25° C.
E179. The aqueous immunogenic composition according to any one of E1 to E174 wherein the percentage of RSV F protein in the prefusion conformation in the composition is higher than about 80% after 3 weeks at 25° C.
E180. The aqueous immunogenic composition according to any one of E1 to E174 wherein the percentage of RSV F protein in the prefusion conformation in the composition is higher than about 85% after 3 weeks at 25° C.
E181. The aqueous immunogenic composition according to any one of E1 to E174 wherein the percentage of RSV F protein in the prefusion conformation in the composition is higher than about 90% after 3 weeks at 25° C.
E182. The aqueous immunogenic composition according to any one of E158 to E181 wherein the percentage of RSV F protein in the prefusion conformation in the composition is measured according to a SEC-Fab titration assay using an antibody binding specifically to the RSV F protein in the prefusion conformation, preferably as disclosed in example 1.
E183. The aqueous immunogenic composition according to any one of E158 to E182 wherein the percentage of RSV F protein in the prefusion conformation in the composition is measured according to an Elisa assay using an antibody binding specifically to the RSV F protein in the prefusion conformation, preferably as disclosed in example 1.
E184. The aqueous immunogenic composition according to E182 or E183 wherein the antibody is AM22.
E185. The aqueous immunogenic composition according to any one of E182 or E183 wherein the antibody is AM14.
E186. The aqueous immunogenic composition according to any one of E1 to E185 for use as a vaccine.
E187. The aqueous immunogenic composition according to any one of E1 to E185 for eliciting an immune response to RSV in a subject.
E188. The aqueous immunogenic composition according to any one of E1 to E185 for reducing or preventing RSV-associated diseases in a subject.
E189. The aqueous immunogenic composition according to any one of E1 to E185 for use in the manufacture of a medicament for reducing or preventing RSV-associated diseases in a subject.
E190. A method of eliciting an immune response to RSV in a subject, comprising administering to the subject an aqueous immunogenic composition according to any one of E1 to E185.
E191. A method of reducing or preventing RSV-associated diseases in a subject, comprising administering to the subject an aqueous immunogenic composition according to any one of E1 to E185
E192. The composition of E186 to E189 or the method of E190 or E191 wherein the subject is a human, preferably a child, a pregnant woman or human of at least 50, 55 or 60 years of age.
E193. A method for storing an aqueous immunogenic composition comprising an RSV F protein trimer in the prefusion conformation, comprising storing the composition at a temperature of at least about 15° C.
E194. The method according to E193 wherein the temperature is between about 15 and about 30° C.
E195. The method according to E194 wherein the temperature is about 15, 16, 17, 18, 19, 20, 21, 22, 23, 24, 25, 26, 27, 28, 29 or 30° C.
E196. The method according to E195 wherein the temperature is about 20° C.
E197. The method according to any one of E193 to E196 wherein the RSV F protein is a F protein of subtype A.
E198. The method according to any one of E193 to E196 wherein the RSV F protein is a F protein of subtype B.
E199. The method according to any one of E193 to E198 wherein the RSV F protein is a mutant of wild type RSV F protein.
E200. The method according to any one of E193 to E199 wherein the RSV F protein displays introduced mutations in the amino acid sequence relative to the amino acid sequence of the corresponding wild-type RSV F protein and is immunogenic against the wild-type RSV F protein in the prefusion conformation or against a virus comprising the wild-type F protein. Said mutations can be for example amino acid substitutions, deletions, or additions relative to a wild-type RSV F protein.
E201. The method according to any one of E193 to E200 wherein the RSV F protein is a mutant of a wild-type RSV F protein, wherein the introduced amino acid mutations are mutation of a pair of amino acid residues in a wild-type RSV F protein to a pair of cysteines. The introduced pair of cysteine residues allows for formation of a disulfide bond between the cysteine residues that stabilize the protein's conformation or oligomeric state, such as the prefusion conformation.
E202. The method according to E201 wherein the RSV F protein comprises one of the following pairs of mutations: 55C and 188C; 155C and 290C; 103C and 148C; and 142C and 371C, such as S55C and L188C; S155C and S290C; A103C and I148C; and L142C and N371C.
E203. The method according to any one of E193 to E202 wherein the RSV F protein comprises amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the prefusion conformation, but exposed to solvent in the postfusion conformation. Examples of the replacement amino acids include large aliphatic amino acids (Ile, Leu and Met) or large aromatic amino acids (His, Phe, Tyr and Trp).
E204. The method according to E203 wherein the RSV F protein comprises a cavity filling mutation selected from the group consisting of:
   (1) substitution of S at positions 55, 62, 155, 190, or 290 with I, Y, L, H, or M;
   (2) substitution of T at position 54, 58, 189, 219, or 397 with I, Y, L, H, or M;
   (3) substitution of G at position 151 with A or H;
   (4) substitution of A at position 147 or 298 with I, L, H, or M;
   (5) substitution of V at position 164, 187, 192, 207, 220, 296, 300, or 495 with I, Y, H; and
   (6) substitution of R at position 106 with W.
E205. The method according to E203 wherein the RSV F protein comprises at least one cavity filling mutation selected from the group consisting of: T54H, S190I, and V296I.
E206. The method according to any one of E193 to E205 wherein the RSV F protein comprises electrostatic mutations, which decrease ionic repulsion or increase ionic attraction between residues in a protein that are proximate to each other in the folded structure.
E207. The method according to E206 wherein the RSV F protein comprises an electrostatic substitution that reduces repulsive ionic interactions or increases attractive ionic interactions with acidic residues of Glu487 and Asp489 from another protomer of RSV F trimer.
E208. The method according to E206 wherein the RSV F protein comprises an electrostatic mutation selected from the group consisting of:
   (1) substitution of E at position 82, 92, or 487 by D, F, Q, T, S, L, or H;
   (2) substitution of K at position 315, 394, or 399 by F, M, R, S, L, I, Q, or T;
   (3) substitution of D at position 392, 486, or 489 by H, S, N, T, or P; and
   (4) substitution of R at position 106 or 339 by F, Q, N, or W.
E209. The method according to any one of E193 to E208 wherein the RSV F protein comprises a combination of two or more different types of mutations selected from engineered disulfide mutations, cavity filling mutations, and electrostatic mutations.
E210. The method according to any one of E193 to E201 wherein the RSV F protein comprises a combination of mutations relative to the corresponding wild-type RSV F protein, wherein the combination of mutations is selected from the group consisting of:
   (1) combination of 103C, 148C, 190I, and 486S, preferably A103C, I148C, S190I, and D486S;
   (2) combination of 54H 55C 188C 486S, preferably T54H S55C L188C D486S;
   (3) combination of 54H, 103C, 148C, 190I, 296I, and 486S, preferably T54H, A103C, I148C, S190I, V296I, and D486S;
   (4) combination of 54H, 55C, 142C, 188C, 296I, and 371C preferably T54H, S55C, L142C, L188C, V296I, and N371C;
   (5) combination of 55C, 188C, and 486S preferably S55C, L188C, and D486S;
   (6) combination of 54H, 55C, 188C, and 190I preferably T54H, S55C, L188C, and S190I;
   (7) combination of 55C, 188C, 190I, and 486S preferably S55C, L188C, S190I, and D486S;
   (8) combination of 54H, 55C, 188C, 190I, and 486S preferably T54H, S55C, L188C, S190I, and D486S;
   (9) combination of 155C, 190I, 290C, and 486S preferably S155C, S190I, S290C, and D486S;
   (10) combination of 54H, 55C, 142C, 188C, 296I, 371C, 486S, 487Q, and 489S preferably T54H, S55C, L142C, L188C, V296I, N371C, D486S, E487Q, and D489S;
   (11) combination of 54H, 155C, 190I, 290C, and 296I preferably T54H, S155C, S190I, S290C, and V296I, and
   (12) combination of 155C, 190F, 290C, and 207L preferably S155C, S190F, S290C, and V207L.
E211. The aqueous immunogenic composition according to any one of E193 to E201 wherein the RSV F protein comprises a combination of mutations relative to the corresponding wild-type RSV F protein, wherein the combination of mutations is selected from the group consisting of:
   (1) combination of 215P and 486N preferably S215P and D486N,
   (2) combination of 66E, 215P, and 486N preferably K66E, S215P, and D486N,
   (3) combination of 66E, 76V, 215P, and 486N preferably K66E, 176V, S215P, and D486N, and,
   (4) combination of 66E, 671, 76V, 215P, and 486N preferably K66E, N671, 176V, S215P, and D486N.
E212. The method according to any one of E193 to E202 wherein the RSV F protein comprises the mutations 103C, 148C, 190I, and 486S, preferably A103C, I148C, S190I, and D486S.
E213. The method according to any one of E193 to E212 wherein the RSV F protein comprises a trimerization domain. The trimerization domain promotes the formation of trimer of three F2-F1 heterodimers. Several exogenous multimerization domains that promote formation of stable trimers of soluble proteins are known in the art. The trimerization domain is preferably selected from the group consisting of:
(1) the GCN4 leucine zipper (Harbury et al. 1993 Science 262:1401-1407); (2) the trimerization motif from the lung surfactant protein (Hoppe et al. 1994 February S Lett 344:191-195); (3) collagen (McAlinden et al. 2003 Biol Chem 278:42200-42207); and (4) the phage T4 fibritin foldon (Miroshnikov et al. 1998 Protein Eng 11:329-414).
E214. The method according to E213 wherein the trimerization domain is the phage T4 fibritin foldon of SEQ ID NO: 7.
E215. The method according to E213 or E214 wherein the trimerization domain is linked to the first RSV protein at the C-terminus of F1 polypeptide.
E216. The method according to E215 wherein the trimerization domain is linked to the first RSV protein at the C-terminus of F1 polypeptide via a linker.
E217. The method according to E216 wherein the linker is selected from GG, GS, or SAIG.
E218. The method according to E217 wherein the trimerization domain is a T4 foldon fibritin domain and is linked to the first RSV protein at the C-terminus of F1 polypeptide via a SAIG linker.
E219. The method according to any one of E193 to E218 wherein the first RSV F protein comprises two separate polypeptide chains, said separate polypeptide chains being the F1 polypeptide and the F2 polypeptide.
E220. The method according to E219 wherein the F2 polypeptide is linked to the F1 polypeptide by one, two, three, four or five disulfide bonds to form a F2-F1 heterodimer.
E221. The method according to any one of E193 to E218 wherein the first RSV F protein is in the form a single chain protein, wherein the F2 polypeptide is linked to the F1 polypeptide by a peptide bond or a linker.
E222. The method according to E221 wherein the linker is selected from G, GG, GGG, GS, or SAIG.
E223. The method according to E221 wherein the linker is full length pep27 sequence or a fragment thereof.
E224. The method according to E219 to E223 wherein the F1 polypeptide of the first RSV F protein lacks the entire cytoplasmic domain or the entire cytoplasmic domain and a portion of or all entire transmembrane domain.
E225. The method according to E219 to E224 wherein amino acid residues 514 through 574 are absent from the F1 polypeptide of the first RSV F protein.
E226. The method according to E219 to E225 wherein the F2 polypeptide chain has the same length as the full-length F2 polypeptide of the corresponding wild-type RSV F protein or has deletions, such as deletions of 1, 2, 3, 4, 5, 6, 7, or 8 amino acid residues from the N-terminus or C-terminus of the F2 polypeptide.
E227. The method according to any one of E193 to E198 wherein the RSV F protein is a RSV protein mutant as disclosed in WO2017/109629 which is hereby incorporated by reference in its entirety.
E228. The aqueous immunogenic composition according to any one of E193 to E198 wherein the RSV F protein is a RSV protein mutant as disclosed in WO2009/079796, WO2010/149745, WO2011/008974, WO2014/160463, WO2014/174018, WO2014/202570, WO2015/013551, WO2015/177312, WO2017/005848, WO2017/174564, WO2017/005844 and WO2018/109220 which are all hereby incorporated by reference in their entirety.
E229. The method according to any one of E193 to E196 wherein the aqueous immunogenic composition is a composition according to any one of E1 to E189.
E230. A method for lyophilizing an aqueous immunogenic composition comprising an RSV F protein trimer in the prefusion conformation comprising the step of freezing the composition wherein said step comprises reducing the temperature to a freezing temperature comprised between −40° C. and −60° C. at a freezing ramp rate of at least about 0.3° C./min.
E231. The method according to E230 wherein the freezing temperature is between about −45° C. and about −55° C.
E232. The method according to E231 wherein the freezing temperature is about −50° C.
E233. The method according to any one of E230 to E232 wherein the composition is maintained at the freezing temperature for at least about 45 mins, for example between 45 mins and 120 mins.
E234. The method according to E233 wherein the composition is maintained at the freezing temperature for at least about 60 mins, for example between 60 mins and 120 mins.
E235. The method according to E233 wherein the composition is maintained at the freezing temperature for about 60 mins.
E236. The method according to any one of E230 to E235 wherein the freezing ramp rate is at least about 0.4, 0.5, 0.6, 0.7, 0.8, 0.9 or 1° C./min, for example at least about 0.4° C./min.
E237. The method according to E237 wherein the freezing ramp rate is at least 1° C./min. For example, the freezing ramp rate is about 1, 1.1, 1.2, 1.3, 1.4, 1.5, 1.6, 1.7, 1.8, 1.9 or 2° C./min.
E238. The method according to any one of E230 to E237 wherein the aqueous immunogenic composition is a composition according to any one of E1 to E189.
E239. A method for lyophilizing an aqueous immunogenic composition comprising an RSV F protein trimer in the prefusion conformation comprising the following steps:
(a) providing an aqueous composition comprising an RSV F protein trimer in the prefusion conformation,
(b) freezing the composition wherein said step comprises reducing the temperature to a freezing temperature comprised between about −40° C. and about −60° C. and at a freezing ramp rate of at least about 0.3° C./min (for example between about 0.3 and about 2° C./min) and, maintaining the composition at the freezing temperature for at least about 30 min, for example between 30 mins and 120 mins.
(c) annealing the frozen composition;
(d) refreezing the annealed composition wherein said step comprises reducing the temperature to a refreezing temperature comprised between about −40° C. and about −60° C. and at a refreezing ramp rate of at least 0.3° C./min, and maintaining the composition at the refreezing temperature for at least about 30 min, for example between 30 mins and 120 mins;
(e) drying the refrozen composition, and
(f) obtaining a lyophilized composition comprising the RSV F protein trimer in the prefusion conformation.
E240. The method according to E239 wherein the freezing temperature in step (b) is between about −45° C. and about −55° C.
E241. The method according to E240 wherein the freezing temperature in step (b) is about 50° C.
E242. The method according to any one of E239 to E241 wherein the composition in step (b) is maintained at the freezing temperature for at least about 45 mins, for example between 45 mins and 120 mins.
E243. The method according to E242 wherein the composition in step (b) is maintained at the freezing temperature for at least about 60 mins, for example between 60 mins and 120 mins.
E244. The method according to any one of E239 to E243 wherein the freezing ramp rate in step (b) is at least about 0.4, 0.5, 0.6, 0.7, 0.8, 0.9 or 1° C./min, for example between about 0.3 and about 2° C./min.
E245. The method according to E244 wherein the freezing ramp rate in step (b) is at least about 0.4° C./min.
E246. The method according to E245 wherein the freezing ramp rate in step (b) is at least about 1° C./min.
E247. The method according to any one of E239 to E246 wherein the annealing step is conducted at an annealing temperature comprised between about-25 and about −5° C.
E248. The method according to E247 wherein the annealing temperature is comprised between about −15° C. and about −5° C.
E249. The method according to E248 wherein the annealing temperature is about −10° C.
E250. The method according to any one of E239 to E249 wherein the freezing temperature is increased to the annealing temperature at a ramp rate comprised between about 0.3 and about 1° C./min.
E251. The method according to any one of E239 to E250 wherein the composition is maintained at the annealing temperature for at least about 60 mins, for example between about 60 mins and 120 mins.
E252. The method according to any one of E239 to E251 wherein the refreezing temperature in step (d) is between about −45° C. and about −55° C.
E253. The method according to E252 wherein the refreezing temperature in step (d) is about −50° C.
E254. The method according to any one of E239 to E253 wherein the composition in step (d) is maintained at the refreezing temperature for at least about 45 mins, for example between about 45 mins and about 120 mins.
E255. The method according to E254 wherein the composition in step (d) is maintained at the refreezing temperature for at least about 60 mins.
E256. The method according to any one of E239 to E255 wherein the annealing temperature is decreased to the refreezing temperature at a refreezing ramp rate comprised between about 0.3 and about 2° C./min.
E257. The method according to E256 wherein the refreezing ramp rate in step (d) is at least about 0.4, 0.5, 0.6, 0.7, 0.8, 0.9 or 1° C./min
E258. The method according to E257 wherein the refreezing ramp rate in step (d) is at least about 0.4° C./min.
E259. The method according to E258 wherein the refreezing ramp rate in step (d) is at least about 1° C./min.
E260. The method according to any one of E239 to E259 wherein step (e) comprises a first drying step conducted at a first drying temperature comprised between about −10° C. and about 20° C.
E261. The method according to E260 wherein the first drying step is conducted at a first drying temperature comprised between −5° C. and 5° C.
E262. The method according to E261 wherein the first drying step is conducted at 0° C.
E263. The method according to any one of E260 to E262 wherein the refreezing temperature is increased to the first drying temperature at a first drying ramp rate comprised between about 0.1° C. and about 1° C./min.
E264. A method according to E263 wherein the first drying ramp rate in step (e) is about 0.5° C./min.
E265. The method according to any one of E260 to E264 wherein the first drying temperature is maintained for at least about 1 min, for example between about 1 min and 30 mins.
E266. The method according to any one of E260 to E265 wherein the first drying step is conducted at a chamber pressure comprised between about 150 mTorr and about 250 mTorr.
E267. The method according to E266 wherein the first drying step is conducted at a chamber pressure of about 200 mTorr.
E268. The method according to any one of E260 to E267 wherein step (e) comprises a second drying step conducted after the first drying step at a second drying temperature comprised between about 40° C. and about 60° C.
E269. The method according to E268 wherein the second drying step is conducted at a temperature comprised between about 45° C. and about 55° C.
E270. The method according to E269 wherein the second drying step is conducted at about 50° C.
E271. The method according to any one of E268 to E270 wherein the first drying temperature is increased to the second drying temperature at a second drying ramp rate comprised between about 0.1 and about 0.5° C./min.
E272. The method according to E271 wherein the second drying ramp rate in step (e) is about 0.2° C./min
E273. The method according to any one of E268 to E272 wherein the second drying temperature is maintained for at least about 400 mins to about 650 mins, preferably between about 500 mins and about 600 mins.
E274. The method according to E273 wherein the second drying temperature is maintained for about 575 mins.
E275. The method according to any one of E268 to E274 wherein the second drying step is conducted at a chamber pressure comprised between about 150 mTorr and about 250 mTorr.
E276. A method according to E275 wherein the second drying step is conducted at a chamber pressure of about 200 mTorr.
E277. The method according to any one of E239 to E276 wherein the volume of aqueous composition provided in step (a) is of between about 0.1 mL and about 10 mL, preferably about 0.1 mL and about 5 mL, more preferably about 0.1 mL and about 1 mL.
E278. The method according to E277 wherein the volume of aqueous composition provided in step (a) is about 0.1, 0.2, 0.3, 0.4, 0.5, 0.6, 0.7, 0.8, 0.9 or 1.0 mL, preferably about 0.5 mL.
E279. The method according to E277 wherein the volume of aqueous composition provided in step (a) is about 1.0, 1.1, 1.2, 1.3, 1.4, 1.5, 1.6, 1.7, 1.8, 1.9 or 2.0 mL, preferably about 1.5 mL.
E280. The method according to any one of E239 to E279 wherein the residual moisture in the lyophilized composition is less than about 2%, preferably less than about 1%.
E281. The method according to E280 wherein the residual moisture in the lyophilized composition is less than about 0.5%.
E282. The method according to any one of E239 to E281 wherein the percentage of RSV F protein in the prefusion conformation in the lyophilized composition is higher than about 65%, 66%, 67%, 68%, 69% or 70% after 8 months at 25° C.
E283. The method according to E282 wherein the percentage of RSV F protein in the prefusion conformation in the lyophilized composition is higher than about 70% after 8 months at 25° C.
E284. The method according to any one of E239 to E283 wherein the percentage of RSV F protein in the prefusion conformation in the lyophilized composition is higher than about 55%, 56%, 57%, 58%, 59% or 60% after 8 months at 40° C.
E285. The method according to E284 wherein the percentage of RSV F protein in the prefusion conformation in the lyophilized composition is higher than about 60% after 8 months at 40° C.
E286. The method according to any one of E282 to E285 wherein the percentage of RSV F protein in the prefusion conformation in the lyophilized composition is measured according to any of the assays disclosed herein.
E287. The method according to any one of E282 to E285 wherein the percentage of RSV F protein in the prefusion conformation in the lyophilized composition is measured according to a SEC-Fab titration assay using an antibody binding specifically to the RSV F protein in the prefusion conformation.
E288. The method according to any one of E282 to E287 wherein the percentage of RSV F protein in the prefusion conformation in the lyophilized composition is measured according to a SEC-Fab titration assay using an antibody binding specifically to the RSV F protein in the prefusion conformation such as AM22 or AM14, preferably AM14.
E289. The method according to any one of E239 to E288 wherein the aqueous immunogenic composition is a composition according to any one of E1 to E189.
E290. A lyophilized immunogenic composition obtained or obtainable by any one of the methods according to E239 to E289.
E291. A lyophilized immunogenic composition obtained by lyophilizing an aqueous immunogenic composition according to any one of E1 to E189.
E292. A lyophilized immunogenic composition comprising 0.08 mg of a first RSV F protein trimer as defined in any of E64 to E96, 0.08 mg of a second RSV F protein trimer as defined in any of E98 to E129, 0.15 mg of Tris base, 1.41 mg Tris-HCl, 15 mg sucrose, 31 mg mannitol, 0.10 mg PS80 and 1.49 mg NaCl.
E293. The lyophilized immunogenic composition according to E290 or E292 wherein the residual moisture is less than about 2%, preferably less than 1%.
E294. The lyophilized immunogenic composition according to E290 or E293 wherein the residual moisture is less than about 0.5%.
E295. The lyophilized immunogenic composition according to any one of E290 to E294 wherein the percentage of RSV F protein in the prefusion conformation is higher than about 65%, 66%, 67%, 68%, 69% or 70% after 8 months at 25° C.
E296. The lyophilized immunogenic composition according to any one of E290 to E294 wherein the percentage of RSV F protein in the prefusion conformation is higher than about 70% after 8 months at 25° C.
E297. The lyophilized immunogenic composition according to any one of E290 to E294 wherein the percentage of RSV F protein in the prefusion conformation is higher than about 55%, 56%, 57%, 58%, 59% or 60% after 8 months at 40° C.
E298. The lyophilized immunogenic composition according to any one of E290 to E294 wherein the percentage of RSV F protein in the prefusion conformation is higher than about 60% after 8 months at 40° C.
E299. The lyophilized immunogenic composition according to any one of E295 to E298 wherein the percentage of RSV F protein in the prefusion conformation is measured according to any of the assay disclosed herein.
E300. The lyophilized immunogenic composition according to any one of E295 to E298 wherein the percentage of RSV F protein in the prefusion conformation is measured according to a SEC-Fab titration assay using an antibody binding specifically to the RSV F protein in the prefusion conformation.
E301. The lyophilized immunogenic composition according to any one of E295 to E300 wherein the percentage of RSV F protein in the prefusion conformation is measured according to a SEC-Fab titration assay using an antibody binding specifically to the RSV F protein in the prefusion conformation such as AM22 or AM14, preferably AM14.
E302. A kit comprising
   i) a lyophilized composition according to any one of E290 to E301, and
   ii) a diluent for reconstituting the lyophilized composition.
E303. The kit of E302 wherein the diluent is water for injection.
E304. The kit of E302 or E303 wherein the volume of the diluent for reconstitution is between about 0.1 mL and about 10 mL, preferably about 0.1 mL and about 5 mL, more preferably about 0.1 mL and about 1 mL.
E305. The kit of E304 wherein the volume of the diluent for reconstitution is of about 0.1, 0.2, 0.3, 0.4, 0.5, 0.6, 0.7, 0.8, 0.9 or 1.0 mL, preferably about 0.50, 0.55, 0.60, 0.65 or 0.70 mL.
E306. A kit comprising,
   i) a lyophilized composition, and
   ii) a diluent comprising sodium chloride at a concentration of between about 20 mM and about 300 mM;
wherein the reconstitution of the lyophilized composition with the diluent results in an aqueous immunogenic composition according to any one of E1 to E189.
E307. The kit of E306 wherein the volume of the diluent for reconstitution is of between about 0.1 mL and about 10 mL, preferably about 0.1 mL and about 5 mL, more preferably about 0.1 mL and about 1 mL.
E308. The kit of E306 or E307 wherein the diluent comprises sodium chloride at a concentration of about 20, 30, 40, 50, 60, 70, 80, 90, 100, 110, 120, 130, 140, 150, 160, 170, 180, 190, 200, 210, 220, 230, 240, 250, 260, 270, 280, 290 or 300 mM.
E309. The kit of E302 to E308 wherein the diluent comprises a preservative.
E310. The kit of E309 wherein the preservative is selected from the group consisting of chlorobutanol, m-cresol, methylparaben, propylparaben, 2-phenoxyethanol, benzethonium chloride, benzalkonium chloride, benzoic acid, benzyl alcohol, phenol, thimerosal and phenylmercuric nitrate.
E311. The kit of E310 wherein the preservative is selected from the group consisting of benzethonium chloride, 2-phenoxyethanol, phenol and thimerosal. In a preferred embodiment, the preservative is 2-phenoxyethanol.
E312. The kit of E311 wherein the preservative is 2-phenoxyethanol.
E313. The kit of any one of E309 to E312 wherein the concentration of preservative in the diluent is selected to achieve a concentration of preservative of at least 9 mg/mL in the aqueous composition resulting from reconstitution of the lyophilized composition with the diluent.
E314. The kit of any one of E309 to E312 wherein the concentration of preservative in the diluent is selected to achieve a concentration of preservative of 10 mg/mL in the aqueous composition resulting from reconstitution of the lyophilized composition with the diluent.

The section headings used herein are for organizational purposes only and are not to be construed as limiting the subject matter described.

### 2. Definitions

As used herein, the term “about” when used to modify a numerically defined parameter means that the parameter may vary by as much as 10% below or above the stated numerical value for that parameter. For example, a dose of about 5 mg means 5 mg+10%, i.e., it may vary between 4.5 mg and 5.5 mg.

The term “F0 polypeptide” (F0) refers to the precursor polypeptide of the RSV F protein, which is composed of a signal polypeptide sequence, a F1 polypeptide sequence, a pep27 polypeptide sequence, and a F2 polypeptide sequence. With rare exceptions the F0 polypeptides of the known RSV strains consist of 574 amino acids.

The term “F1 polypeptide” (F1) refers to a polypeptide chain of a mature RSV F protein. Native F1 includes approximately residues 137-574 of the RSV F0 precursor and is composed of (from N- to C-terminus) an extracellular region (approximately residues 137-524), a transmembrane domain (approximately residues 525-550), and a cytoplasmic domain (approximately residues 551-574). As used herein, the term encompasses both native F1 polypeptides and F1 polypeptides including modifications (e.g., amino acid substitutions, insertions, or deletions) from the native sequence, for example, modifications designed to stabilize an RSV F protein mutant or to enhance the immunogenicity of an RSV F protein mutant. The term “F2 polypeptide” (F2) refers to the polypeptide chain of a mature RSV F protein. Native F2 includes approximately residues 26-109 of the RSV F0 precursor. As used herein, the term encompasses both native F2 polypeptides and F2 polypeptides including modifications (e.g., amino acid substitutions, insertions, or deletions) from the native sequence, for example, modifications designed to stabilize an RSV F protein mutant in a prefusion conformation or to enhance the immunogenicity of an RSV F protein mutant. In native RSV F protein, the F2 polypeptide is linked to the F1 polypeptide by two disulfide bonds to form a F2-F1 heterodimer.

The term “foldon” or “foldon domain” refers to an amino acid sequence that is capable of forming trimers. One example of such foldon domains is the peptide sequence derived from bacteriophage T4 fibritin, which has the sequence of GYIPEAPRDGQAYVRKDGEWVLLSTFL (SEQ ID NO:7).

The term “AM14” refers to an antibody described in WO 2008/147196 A2, which has a heavy chain variable domain comprising an amino acid sequence of SEQ ID NO:10 and a light chain variable domain comprising an amino acid sequence of SEQ ID NO:11.

The term “AM22” refers to an antibody described in WO 2011/043643 A1, which has a heavy chain variable domain comprising an amino acid sequence of SEQ ID NO: 12 and a light chain variable domain comprising an amino acid sequence of SEQ ID NO:13.

The term “D25” refers to an antibody described in WO 2008/147196 A2, which has a heavy chain variable domain comprising an amino acid sequence of SEQ ID NO:8 and a light chain variable domain comprising an amino acid sequence of SEQ ID NO:9.

The term “prefusion conformation” refers to a structural conformation adopted by an RSV F protein or mutant thereof that can be specifically bound by (i) antibody D25 or AM22 when the RSV F protein or mutant is in the form of a monomer or trimer, or (ii) by antibody AM14 when the RSV F protein mutant is in the form of a trimer. The prefusion trimer conformation is a subset of prefusion conformations.

The term “postfusion conformation” refers to a structural conformation adopted by the RSV F protein that is not specifically bound by D25, AM22, or AM14. Native F protein adopts the postfusion conformation subsequent to the fusion of the virus envelope with the host cellular membrane. RSV F protein may also assume the postfusion conformation outside the context of a fusion event, for example, under stress conditions such as heat and low osmolality, when extracted from a membrane, when expressed as an ectodomain, or upon storage.

The term “soluble protein” refers to a protein capable of dissolving in aqueous liquid and remaining dissolved. The solubility of a protein may change depending on the concentration of the protein in the water-based liquid, the buffering condition of the liquid, the concentration of other solutes in the liquid, for example salt and protein concentrations, and the temperature of the liquid.

The term “vaccine” refers to a pharmaceutical composition comprising an immunogen that is capable of eliciting a prophylactic or therapeutic immune response in a subject. Typically, a vaccine elicits an antigen-specific immune response to an antigen of a pathogen, for example a viral pathogen.

The term “immunogenic” refers to the ability of a substance to cause, elicit, stimulate, or induce an immune response against a particular antigen, in an animal, whether in the presence or absence of an adjuvant. Immunogenicity can be measured by any method or assay known in the art, such as for example animal vaccination models, serum bactericidal assays (SBA), flow cytometry, and in vitro potency assays.

The term “immune response” refers to any detectable response of a cell or cells of the immune system of a host mammal to a stimulus (such as an immunogen), including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypeptide) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells.

The term “immune response” also encompasses any detectable response to a particular substance (such as an antigen or immunogen) by one or more components of the immune system of a vertebrate animal in vitro. The term “immunogen” refers to a compound, composition, or substance that is immunogenic as defined herein below.

The term ‘immunogenic composition” refers to a composition comprising an immunogen.

The term “mutant” of a wild-type RSV F protein, “mutant” of a RSV F protein, “RSV F protein mutant,” or “modified RSV F protein” refers to a polypeptide that displays introduced mutations relative to a wild-type F protein and is immunogenic against the wild-type F protein.

The term “mutation” refers to deletion, addition, or substitution of amino acid residues in the amino acid sequence of a protein or polypeptide as compared to the amino acid sequence of a reference protein or polypeptide. Throughout the specification and claims, the substitution of an amino acid at one particular location in the protein sequence is referred to using a notation “(amino acid residue in wild type protein) (amino acid position) (amino acid residue in engineered protein)”. For example, a notation Y75A refers to a substitution of a tyrosine (Y) residue at the 75th position of the amino acid sequence of the reference protein by an alanine (A) residue (in a mutant of the reference protein). In cases where there is variation in the amino acid residue at the same position among different wild-type sequences, the amino acid code preceding the position number may be omitted in the notation, such as “75A.”

The term “native” or “wild-type” protein, sequence, or polypeptide refers to a naturally existing protein, sequence, or polypeptide that has not been artificially modified by selective mutations.

The term “pep27 polypeptide” or “pep27” refers to a 27-amino acid polypeptide that is excised from the F0 precursor during maturation of the RSV F protein. The sequence of pep27 is flanked by two furin cleavage sites that are cleaved by a cellular protease during F protein maturation to generate the F1 and F2 polypeptides.

The term “specifically bind,” in the context of the binding of an antibody to a given target molecule, refers to the binding of the antibody with the target molecule with higher affinity than its binding with other tested substances. For example, an antibody that specifically binds to the RSV F protein in prefusion conformation is an antibody that binds RSV F protein in prefusion conformation with higher affinity than it binds to the RSV F protein in the postfusion conformation.

As used herein, the term “buffer” refers to an added composition that allows a liquid antibody formulation to resist changes in pH, typically by action of its acid-base conjugate components. When a concentration of a buffer is referred to, it is intended that the recited concentration represent the molar concentration of the free acid or free base form of the buffer.

### 3. The RSV F Protein

The RSV F protein to be included in the aqueous immunogenic composition disclosed herein can be any RSV F protein in the prefusion conformation.

In some embodiments, the RSV F protein is an RSV F protein of subtype A. In some embodiments, the RSV F protein is an RSV F protein of subtype B. In some embodiments, the RSV F protein is a mutant of wild type RSV F protein. In some embodiments, the RSV F protein is a mutant of wild type RSV F protein of subtype A. In some embodiments, the RSV F protein is a mutant of wild type RSV F protein of subtype B. In some embodiments, the mutants display introduced mutations in the amino acid sequence relative to the amino acid sequence of the corresponding wild-type RSV F protein and are immunogenic against the wild-type RSV F protein in the prefusion conformation or against a virus comprising the wild-type F protein. The amino acid mutations in the mutants include amino acid substitutions, deletions, or additions relative to a wild-type RSV F protein.

In some embodiments, the RSV F protein is an RSV protein mutant as disclosed in WO2017/109629 which is hereby incorporated by reference in its entirety.

In some embodiments, the RSV F protein is a mutant of a wild-type RSV F protein, wherein the introduced amino acid mutations are mutation of a pair of amino acid residues in a wild-type RSV F protein to a pair of cysteines (“engineered disulfide mutation”). The introduced pair of cysteine residues allows for formation of a disulfide bond between the cysteine residues that stabilize the protein's conformation or oligomeric state, such as the prefusion conformation. Examples of specific pairs of such mutations include: 55C and 188C; 155C and 290C; 103C and 148C; and 142C and 371C, such as S55C and L188C; S155C and S290C; A103C and I148C; and L142C and N371C.

In still other embodiments, the RSV F protein mutants comprise amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the prefusion conformation, but exposed to solvent in the postfusion conformation. Examples of the replacement amino acids include large aliphatic amino acids (Ile, Leu and Met) or large aromatic amino acids (His, Phe, Tyr and Trp). In some specific embodiments, the RSV F protein mutant comprises a cavity filling mutation selected from the group consisting of:
(1) substitution of S at positions 55, 62, 155, 190, or 290 with I, Y, L, H, or M;
(2) substitution of T at position 54, 58, 189, 219, or 397 with I, Y, L, H, or M;
(3) substitution of G at position 151 with A or H;
(4) substitution of A at position 147 or 298 with I, L, H, or M;
(5) substitution of V at position 164, 187, 192, 207, 220, 296, 300, or 495 with I, Y, H; and
(6) substitution of R at position 106 with W.

In some particular embodiments, the RSV F protein mutant comprises at least one cavity filling mutation selected from the group consisting of: T54H, S190I, and V296I.

In still other embodiments, the RSV F protein mutants comprise electrostatic mutations, which decrease ionic repulsion or increase ionic attraction between residues in a protein that are proximate to each other in the folded structure. In several embodiments, the RSV F protein mutant includes an electrostatic substitution that reduces repulsive ionic interactions or increases attractive ionic interactions with acidic residues of Glu487 and Asp489 from another protomer of RSV F trimer. In some specific embodiments, the RSV F protein mutant comprises an electrostatic mutation selected from the group consisting of:
(1) substitution of E at position 82, 92, or 487 by D, F, Q, T, S, L, or H;
(2) substitution of K at position 315, 394, or 399 by F, M, R, S, L, I, Q, or T;
(3) substitution of D at position 392, 486, or 489 by H, S, N, T, or P; and
(4) substitution of R at position 106 or 339 by F, Q, N, or W.

In still other embodiments, the RSV F protein mutants comprise a combination of two or more different types of mutations selected from engineered disulfide mutations, cavity filling mutations, and electrostatic mutations. In some particular embodiments, the RSV F protein mutants comprise a combination of mutations relative to the corresponding wild-type RSV F protein, wherein the combination of mutations is selected from the group consisting of:
(1) combination of A103C, I148C, S190I, and D486S;
(2) combination of T54H S55C L188C D486S;
(3) combination of T54H, A103C, I148C, S190I, V296I, and D486S;
(4) combination of T54H, S55C, L142C, L188C, V296I, and N371C;
(5) combination of S55C, L188C, and D486S;
(6) combination of T54H, S55C, L188C, and S190I;
(7) combination of S55C, L188C, S190I, and D486S;
(8) combination of T54H, S55C, L188C, S190I, and D486S;
(9) combination of S155C, S190I, S290C, and D486S;
(10) combination of T54H, S55C, L142C, L188C, V296I, N371C, D486S, E487Q, and D489S;
(11) combination of T54H, S155C, S190I, S290C, and V296I, and,
(12) combination of S155C, S190F, S290C, and V207L.

In some embodiments, the RSV F protein is of subtype A and comprises the mutations S155C, S190F, S290C, and V207L.

In some embodiments, the RSV F protein is of subtype B and comprises the mutations S155C, S190F, S290C, and V207L.

In some embodiments, the RSV F protein is of subtype A and comprises the mutations A103C, I148C, S190I, and D486S.

In some embodiments, the RSV F protein is of subtype B and comprises the mutations A103C, I148C, S190I, and D486S.

In some embodiments, the RSV F protein to be included in the composition disclosed herein comprises a trimerization domain. In some embodiments, the trimerization domain promotes the formation of trimer of three F2-F1 heterodimers.

Several exogenous multimerization domains that promote formation of stable trimers of soluble proteins are known in the art. Examples of such multimerization domains that can be linked to a mutant provided by the present disclosure include: (1) the GCN4 leucine zipper (Harbury et al. 1993 Science 262:1401-1407); (2) the trimerization motif from the lung surfactant protein (Hoppe et al. 1994 February S Lett 344:191-195); (3) collagen (McAlinden et al. 2003 Biol Chem 278:42200-42207); and (4) the phage T4 fibritin foldon (Miroshnikov et al. 1998 Protein Eng 11:329-414). In some embodiments, a foldon domain is linked to a F mutant at the C-terminus of F1 polypeptide. In specific embodiments, the foldon domain is a T4 fibritin foldon domain, such as the amino acid sequence GYIPEAPRDGQAYVRKDGEWVLLSTFL (SEQ ID NO: 7).

Typically, the multimerization domain is positioned C-terminal to the F1 polypeptide. It may join directly to the F1 polypeptide chain. Optionally, the multimerization domain is connected to the F1 polypeptide via a linker, such as an amino acid linker, for example the sequence GG, GS, or SAIG. The linker can also be a longer linker (for example, including the repeat sequence GG). Numerous conformationally neutral linkers are known in the art that can be used in the mutants provided by the present disclosure. In some embodiments, the F mutant comprising a foldon domain include a protease cleavage site for removing the foldon domain from the F1 polypeptide, such as a thrombin site between the F1 polypeptide and the foldon domain. In a preferred embodiment, the RSV F protein to be included in the composition disclosed herein comprises a T4 fibritin foldon domain linked at the C-terminus of the F1 polypeptide by an SAIG linker.

In view of the substantial conservation of RSV F sequences, a person of ordinary skill in the art can easily compare amino acid positions between different native RSV F sequences to identify corresponding RSV F amino acid positions between different RSV strains and subtypes. For example, across nearly all identified native RSV F0 precursor proteins, the furin cleavage sites fall in the same amino acid positions. Thus, the conservation of native RSV F protein sequences across strains and subtypes allows use of a reference RSV F sequence for comparison of amino acids at particular positions in the RSV F protein. For the purposes of this disclosure (unless context indicates otherwise), the RSV F protein amino acid positions are given with reference to the amino acid sequence of the full length native F precursor polypeptide of the RSV A2 strain; corresponding to GenInfo Identifier GI 138251 and Swiss Prot identifier P03420.

In some embodiments, the RSV F protein is in the mature form of the RSV F protein, which comprises two separate polypeptide chains, namely the F1 polypeptide and F2 polypeptide. In some other embodiments, the F2 polypeptide is linked to the F1 polypeptide by one or two disulfide bonds to form a F2/F1 heterodimer. In still other embodiments, the RSV F mutants are in the form a single chain protein, wherein the F2 polypeptide is linked to the F1 polypeptide by a peptide bond or peptide linker. Any suitable peptide linkers for joining two polypeptide chains together may be used. Examples of such linkers include G, GG, GGG, GS, and SAIG linker sequences. The linker may also be the full length pep27 sequence or a fragment thereof.

The F1 polypeptide chain of the mutant may be of the same length as the full length F1 polypeptide of the corresponding wild-type RSV F protein; however, it may also have deletions, such as deletions of 1 up to 60 amino acid residues from the C-terminus of the full-length F1 polypeptide. A full-length F1 polypeptide of the RSV F mutants corresponds to amino acid positions 137-574 of the native RSV F0 precursor, and includes (from N- to C-terminus) an extracellular region (residues 137-524), a transmembrane domain (residues 525-550), and a cytoplasmic domain (residues 551-574). It should be noted that amino acid residues 514 onwards in a native F1 polypeptide sequence are optional sequences in a F1 polypeptide of the RSV F protein to be included in the immunogenic composition provided herein, and therefore may be absent from the F1 polypeptide of the mutant.

In some embodiments, the F1 polypeptide of the RSV F mutants lacks the entire cytoplasmic domain. In other embodiments, the F1 polypeptide lacks the cytoplasmic domain and a portion of or all entire transmembrane domain. In some specific embodiments, the mutant comprises a F1 polypeptide wherein the amino acid residues from position 510, 511, 512, 513, 514, 515, 520, 525, or 530 through 574 are absent. Typically, for mutants that are linked to trimerization domain, such as a foldon, amino acids 514 through 754 can be absent. Thus, in some specific embodiment, amino acid residues 514 through 574 are absent from the F1 polypeptide of the mutant. In still other specific embodiments, the F1 polypeptide of the RSV F mutants comprises or consists of amino acid residues 137-513 of a native F0 polypeptide sequence, such as any of alternative F0 precursor sequence such as those disclosed in SEQ ID Nos: 1, 2, 4, 6, and 81-270 of WO2017109629.

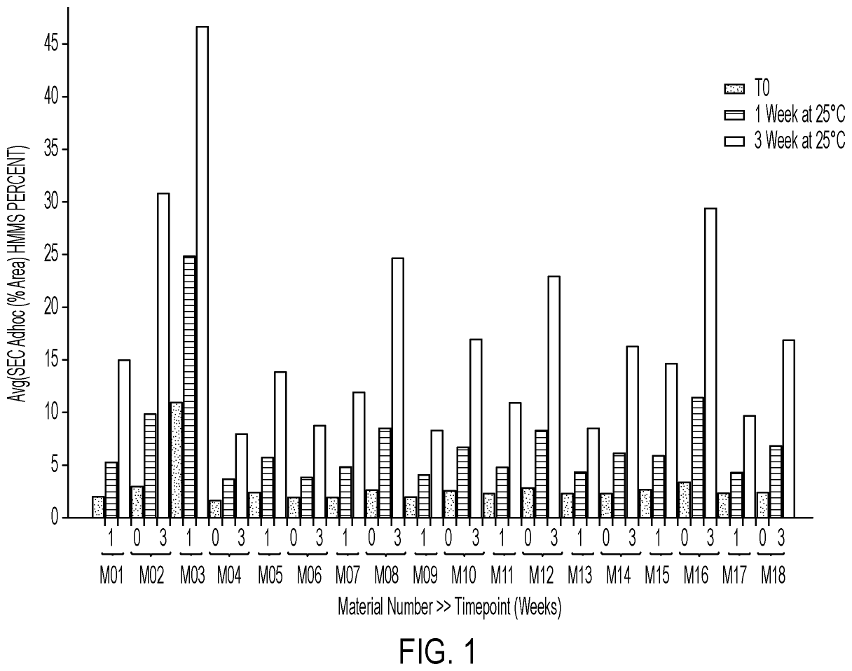

On the other hand, the F1 polypeptide of the RSV F mutant may include a C-terminal linkage to a trimerization domain, such as a foldon. Many of the sequences of the RSV F mutants disclosed herein include a sequence of protease cleavage site, such as thrombin cleavage site (LVPRGS), protein tags, such as 6× His-tag (HHHHHH) and Streptag II (WSHPGFEK), or linker sequences (such as GG and GS) (See FIG. 1) that are not essential for the function of the RSV

F protein, such as for induction of an immune response. A person skilled in the art will recognize such sequences, and when appropriate, understand that these sequences are not included in a disclosed RSV F mutant.

In the RSV F mutants provided by the present disclosure, the F2 polypeptide chain may be of the same length as the full-length F2 polypeptide of the corresponding wild-type RSV F protein; it may also have deletions, such as deletions of 1, 2, 3, 4, 5, 6, 7, or 8 amino acid residues from the N-terminus or C-terminus of the F2 polypeptide.

The mutant in F0 form (i.e., a single chain polypeptide comprising the F2 polypeptide joined to the F1 polypeptide with or without partial or full-length pep 27) or F2-F1 heterodimer form may form a protomer. The mutant may also be in the form of a trimer, which comprises three of the same protomer. Further, the mutants may be glycosylated proteins (i.e., glycoproteins) or non-glycosylated proteins. The mutant in F0 form may include, or may lack, the signal peptide sequence.

The F1 polypeptide and F2 polypeptide of the RSV F protein mutants to which one or more mutations are introduced can be from any wild-type RSV F proteins known in the art or discovered in the future, including, without limitations, the F protein amino acid sequence of RSV subtype A, and subtype B strains, including A2 Ontario and Buenos Aires, or any other subtype. In some embodiments, the RSV F mutant comprises a F1 and/or a F2 polypeptide from a RSV A virus, for example, a F1 and/or F2 polypeptide from a RSV F0 precursor protein set forth in any one of SEQ ID NOs: 1, 2, 4, 6, and 81-270 of WO2017109629 to which one or more mutations are introduced. In some other embodiments, the RSV F mutant comprises a F1 and/or a F2 polypeptide from a RSV B virus, for example, a F1 and/or F2 polypeptide from a RSV F0 precursor protein set forth in any one of SEQ ID NOs: 2, and 211-263 of WO2017/109629 to which one or more mutations are introduced. In still other embodiments, the RSV F mutant comprises a F1 and/or a F2 polypeptide from a RSV bovine virus, for example, a F1 and/or F2 polypeptide from a RSV F0 precursor protein set forth in any one of SEQ ID NOs: 264-270 of WO2017109629 to which one or more mutations are introduced.

In some embodiments, the RSV F protein is an RSV protein mutant as disclosed WO2009/079796, WO2010/149745, WO2011/008974, WO2014/160463, WO2014/174018, WO2014/202570, WO2015/013551, WO2015/177312, WO2017/005848, WO2017/174564, WO2017/005844 and WO2018/109220. The RSV F proteins disclosed in these references are hereby incorporated by reference in their entirety.

In a preferred embodiment, the RSV F protein comprises an F1 polypeptide of SEQ ID NO: 1 and an F2 polypeptide of SEQ ID NO: 2

In a preferred embodiment, the RSV F protein comprises an F1 polypeptide of SEQ ID NO: 3 and an F2 polypeptide of SEQ ID NO: 4

The RSV F protein included in the aqueous immunogenic formulation disclosed herein can be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Examples of suitable mammalian cells include Chinese hamster ovary (CHO) cells, human embryonic kidney cells (HEK293 or Expi 293 cells, typically transformed by sheared adenovirus type 5 DNA), NIH-3T3 cells, 293-T cells, Vero cells, and Hela cells. Suitable avian cells include, for example, chicken embryonic stem cells (e.g., EBx® cells), chicken embryonic fibroblasts, chicken embryonic germ cells, quail fibroblasts (e.g. ELL-O), and duck cells. Suitable insect cell expression systems, such as baculovirus-vectored systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668. Similarly, bacterial and mammalian cell expression systems are also known in the art and described in, e.g., Yeast Genetic Engineering (Barr et al., eds., 1989) Butterworths, London.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used. The RSV F protein to be included in the composition disclosed herein can also be prepared according to the methods disclosed in WO2022/070129.

The RSV F protein used in the compositions disclosed herein can be purified using any suitable methods. For example, methods for purifying RSV F protein mutant polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the RSV F protein mutant polypeptides can include a “tag” that facilitates purification, such as an epitope tag or a histidine (HIS) tag. Such tagged polypeptides can conveniently be purified, for example from conditioned media, by chelating chromatography or affinity chromatography. The RSV F protein to be included in the composition disclosed herein can also be prepared according to the methods disclosed in WO2020/026147, WO2022/023895 and WO2022/023896.

### 4. Immunogenic Aqueous Compositions Comprising an RSV F Protein Trimer in the Prefusion Conformation

The aqueous immunogenic compositions of the invention are disclosed in the above embodiments of section 1.

As demonstrated in the examples, the presence of sodium chloride and at least one of sucrose, mannitol and glycine within the disclosed concentration ranges in aqueous composition comprising an RSV F protein trimer in the prefusion conformation is advantageous as it helps to stabilize said protein in the required form (trimer in the prefusion conformation) and also limit the presence of HMMS in the composition.

For example, as shown in Examples 2 to 5, the presence of sodium chloride reduces the percentage of aggregated protein in the aqueous immunogenic composition.

Also, as shown by Example 2, the presence of sucrose in the composition helps to preserve prefusion conformation content and reduced the increase in % HMMS.

The examples also suggest that a pH range of about 7.0 to 8.0 is suitable to minimize loss of prefusion content and increases in % HMMS. In particular, Example 4 suggests that aggregation increases at pH less than 6.5 or higher than 8.0.

In certain embodiments, the immunogenic composition of the invention comprises a surfactant. A surfactant (or a surface-active agent) is generally defined as (a) a molecule or compound comprising a hydrophilic group or moiety and a lipophilic (hydrophobic) group or moiety and/or (b) a molecule, substance or compound that lowers or reduces surface tension of a solution. As defined herein, a “surfactant” of the present invention is any molecule or compound that lowers the surface tension of an immunogenic composition.

A surfactant used in a composition as disclosed herein comprises any surfactant or any combination of surfactants which stabilizes and inhibits aggregation of an immunogenic composition described herein. Thus, a surfactant for use in the composition disclosed herein includes, but is not limited to, polysorbate 20 (Tween™20), polysorbate 40 (Tween™40), polysorbate 60 (Tween™60), polysorbate 65 (Tween™65), polysorbate 80 (Tween™80), polysorbate 85 (Tween™85), Triton™ N-101, Triton™ X-100, oxtoxynol 40, nonoxynol-9, triethanolamine, triethanolamine polypeptide oleate, polyoxyethylene-660 hydroxystearate (PEG-15, Solutol H15), polyoxyethylene-35-ricinoleate (Cremophor ELT), soy lecithin, poloxamer, hexadecylamine, octadecylamine, octadecyl amino acid esters, lysolecithin, dimethyl-dioctadecylammonium bromide, methoxyhexadecylgylcerol, pluronic polyols, polyamines (e.g., pyran, dextransulfate, poly IC, carbopol), peptides (e.g., muramyl peptide and dipeptide, dimethylglycine, tuftsin), oil emulsions, mineral gels (e.g., aluminum phosphate) and immune stimulating complexes (ISCOMS).

A person of skill in the art may readily determine a suitable surfactant or surfactant combination by measuring the surface tension of a particular immunogenic composition formulation in the presence and absence of the surfactant(s). Alternatively, a surfactant is evaluated qualitatively (e.g., visual inspection of particulate formation) or quantitatively (e.g., light scattering, sedimentation velocity centrifugation, optical density, antigenicity) for its ability to reduce, inhibit or prevent aggregation of an immunogenic composition.

In some embodiments, the surfactant is a polysorbate. In some embodiments, the polysorbate is polysorbate 20 or polysorbate 80. In some embodiments, the polysorbate is polysorbate 80. In some embodiments, the concentration of the surfactant is of about 0.01 mg/mL and about 10 mg/mL.

The concentration of the surfactant generally ranges from about 0.01 mg/ml to about 10 mg/ml, from about 0.01 mg/ml to about 5.0 mg/ml, from about 0.01 mg/ml to about 2.0 mg/ml, from about 0.01 mg/ml to about 1.5 mg/ml, from about 0.01 mg/ml to about 01.0 mg/ml, from about 0.01 mg/ml to about 0.5 mg/ml, from about 0.01 mg/ml to about 0.4 mg/ml, from about 0.01 mg/ml to about 0.3 mg/ml, from about 0.01 mg/ml to about 0.2 mg/ml, from about 0.01 mg/ml to about 0.15 mg/ml, from about 0.01 mg/ml to about 0.1 mg/ml, or from about 0.01 mg/ml, to about 0.05 mg/ml. Further preferably the concentration of the surfactant is about 0.5 mg/ml, about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about 0.14 mg/ml, about 0.15 mg/ml, about 0.16 mg/ml, about 0.17 mg/ml, about 0.18 mg/ml, about 0.19 mg/ml, or about 0.2 mg/ml. Preferably, the concentration of the surfactant is of about 0.01 mg/mL and about 0.05 mg/mL. Most preferably, the concentration of the surfactant is about 0.02 mg/mL

In certain embodiments, the immunogenic composition of the invention comprises an adjuvant. An adjuvant is a substance that enhances the immune response when administered together with an immunogen or antigen. A number of cytokines or lymphokines have been shown to have immune modulating activity, and thus may be used as adjuvants, including, but not limited to, the interleukins 1-α, 1-β, 2, 4, 5, 6, 7, 8, 10, 12 (see, e.g., U.S. Pat. No. 5,723,127), 13, 14, 15, 16, 17 and 18 (and its mutant forms), the interferons-α, β and γ, granulocyte-macrophage colony stimulating factor (GMCSF, see, e.g., U.S. Pat. No. 5,078,996 and ATCC Accession Number 39900), macrophage colony stimulating factor (MCSF), granulocyte colony stimulating factor (GCSF), and the tumor necrosis factors α and β (TNF). Still other adjuvants useful in this invention include chemokines, including without limitation, MCP-1, MIP-1a, MIP-1B, and RANTES.

In certain embodiments, an adjuvant used to enhance an immune response of an immunogenic composition formulation includes, without limitation, MPL™ (3-O-deacylated monophosphoryl lipid A: Corixa, Hamilton, MT), which is described in U.S. Pat. No. 4,912,094, which is hereby incorporated by reference in its entirety. Also suitable for use as adjuvants are synthetic lipid A analogs or aminoalkyl glucosamine phosphate compounds (AGP), or derivatives or analogs thereof, which are available from Corixa (Hamilton, MT), and which are described in U.S. Pat. No. 6,113,918, which is hereby incorporated by reference in its entirety. One such AGP is 2-[(R)-3-Tetradeca11noyloxytetradecanoylamino]ethyl 2-Deoxy-4-O-phosphono-3-O—[(R)-3-tetradecanoyoxytetradecanoyl]-2-[(R)-3-tetradecanoyloxytetradecanoyl-amino]-b-D-glucopyranoside, which is also known as 529 (formerly known as RC529). This 529 adjuvant is formulated as an aqueous form or as a stable emulsion (RC529-SE).

Still other adjuvants include mineral oil and water emulsions, aluminum salts (alum), such as aluminum hydroxide, aluminum phosphate, aluminum sulfate etc., Amphigen, Avridine, L121/squalene, D-lactide-polylactide/glycoside, pluronic polyols, muramyl dipeptide, killed Bordetella, saponins, such as Stimulon™ QS-21 (Antigenics, Framingham, MA.) described in U.S. Pat. No. 5,057,540, which is hereby incorporated by reference in its entirety, and particles generated therefrom such as ISCOMS (immunostimulating complexes), adjuvant systems comprising combination of immunostimulant such as MPL™ and QS-21 in liposomes such as AS01, as described in chapter 14 of Immunopotentiators in Modern Vaccines, p. 265-285, 2017 Elsevier, which is hereby incorporated by reference in its entirety, or ALFQ described in U.S. Pat. No. 10,434,167 which is hereby incorporated by reference in its entirety, ISCOMATRIX (CSL Limited, Parkville, Australia), described in U.S. Pat. No. 5,254,339, Mycobacterium tuberculosis, bacterial lipopolysaccharides, synthetic polynucleotides such as oligonucleotides containing a CpG motif (U.S. Pat. No. 6,207,646, which is hereby incorporated by reference in its entirety), IC-31 (Intercell AG, Vienna, Austria), described in European Patent Nos. 1,296,713 and 1,326,634, a pertussis toxin (PT), or an E. coli heat-labile toxin (LT), particularly LT-K63, LT-R72, PT-K9/G129; see, e.g., International Patent Publication Nos. WO 93/13302 and WO 92/19265, hereby incorporated by reference in its entirety.

Also useful as adjuvants (and carrier proteins) are cholera toxins and mutants thereof, including those described in published International Patent Application number WO 00/18434 (wherein the glutamic acid at amino acid position 29 is replaced by another amino acid (other than aspartic acid), preferably a histidine). Similar CT toxins or mutants are described in published International Patent Application number WO 02/098368 (wherein the isoleucine at amino acid position 16 is replaced by another amino acid, either alone or in combination with the replacement of the serine at amino acid position 68 by another amino acid; and/or wherein the valine at amino acid position 72 is replaced by another amino acid). Other CT toxins are described in published International Patent Application number WO 02/098369 (wherein the arginine at amino acid position 25 is replaced by another amino acid; and/or an amino acid is inserted at amino acid position 49; and/or two amino acids are inserted at amino acid positions 35 and 36).

In certain embodiments, the aqueous immunogenic composition of the invention comprises a preservative. A preservative is a substance conferring resistance to one or more micro-organisms and is useful for example producing multi-dose vaccine formulations having advantageous properties with respect to long term stability of the different antigenic determinants in the immunogenic composition of choice.

In certain embodiments, the preservative is selected from the group consisting of chlorobutanol, m-cresol, methylparaben, propylparaben, 2-phenoxyethanol, benzethonium chloride, benzalkonium chloride, benzoic acid, benzyl alcohol, phenol, thimerosal and phenylmercuric nitrate. In certain embodiments, the preservative is selected from the group consisting of benzethonium chloride, 2-phenoxyethanol, phenol and thimerosal. In a preferred embodiment, the preservative is 2-phenoxyethanol.

In certain embodiments, the concentration of preservative in the aqueous immunogenic composition is at least 9 mg/mL. In one embodiment, the concentration of the preservative in the aqueous immunogenic composition is 10 mg/mL.

### 5. Lyophilized Immunogenic Compositions Comprising an RSV F Protein Trimer in the Prefusion Conformation

The lyophilized immunogenic compositions of the invention are disclosed in the embodiments of above section 1. Such lyophilized compositions can be obtained by lyophilization of the aqueous immunogenic compositions disclosed herein disclosed using lyophilization methods known to the skilled person or preferably using the lyophilization methods disclosed herein (see section 1 and Examples 6 and 7).

The lyophilized immunogenic composition can be reconstituted with any suitable diluent. In certain embodiments, the diluent is water for injection. In certain embodiments, the diluent comprises sodium chloride, preferably at a concentration of between about 20 mM and about 300 mM.

In certain embodiments, the diluent comprises a preservative. In certain embodiments, the preservative is selected from the group consisting of chlorobutanol, m-cresol, methylparaben, propylparaben, 2-phenoxyethanol, benzethonium chloride, benzalkonium chloride, benzoic acid, benzyl alcohol, phenol, thimerosal and phenylmercuric nitrate. In certain embodiments, the preservative is selected from benzethonium chloride, 2-phenoxyethanol, phenol and thimerosal. In a preferred embodiment, the preservative is 2-phenoxyethanol.

In certain embodiments, the concentration of preservative in the diluent is selected to achieve a concentration of at least 9 mg/mL in the reconstituted composition. In one embodiment, the concentration of the preservative in the diluent is selected to achieve a concentration of preservative of 10 mg/mL in the reconstituted composition.

In certain embodiments, the volume of diluent to be used for reconstitution of the lyophilized composition is the same as fill volume (volume of the composition pre-lyophilization).

In certain embodiments, the volume of diluent to be used for reconstitution of the lyophilized composition is the lower than the fill volume. In certain embodiments, the volume of diluent to be used for reconstitution of the lyophilized composition is the greater than the fill volume. In particular, the fill volume and the volume of diluent for reconstitution can be adjusted to modulate the osmolarity of the aqueous composition resulting from the reconstitution of the lyophilized composition with the diluent.

### 6. Use of the Aqueous Immunogenic Compositions Comprising an RSV F Protein Trimer in the Prefusion Conformation

The present disclosure also relates to uses of aqueous immunogenic compositions comprising a RSV F protein trimer in the prefusion conformation as disclosed in section 1 as a vaccine.

In several embodiments, the present disclosure provides a method of eliciting an immune response to RSV in a subject, comprising administering to the subject an aqueous immunogenic compositions comprising a RSV F protein trimer in the prefusion conformation as disclosed herein.

In some particular embodiments, the present disclosure provides a method of reducing or preventing RSV-associated diseases in a subject, comprising administering to the subject an aqueous immunogenic composition comprising RSV F protein trimer in the prefusion conformation as disclosed herein.

In some embodiments, the subject is a human. In some particular embodiments, the human is a child, such as an infant. In some other particular embodiments, the human is woman, particularly a pregnant woman. In some other particular embodiments, the human is at least 50, 55 or 60 years old.

Subjects that can be selected for prophylaxis include those that are at risk for developing an RSV infection because of exposure or the possibility of exposure to RSV. Because nearly all humans are infected with RSV by the age of 2, the entire birth cohort is included as a relevant population for immunization. This could be done, for example, by beginning an immunization regimen anytime from birth to 6 months of age, from 6 months of age to 5 years of age, in pregnant women (or women of child-bearing age) to protect their infants by passive transfer of antibody, family members of newborn infants or those still in utero, and subjects greater than 50 years of age. Subjects at greatest risk of RSV infection with severe symptoms (e.g. requiring hospitalization) include children with prematurity, bronchopulmonary dysplasia, and congenital heart disease.

Administration of the compositions provided by the present disclosure, such as can be carried out using standard routes of administration. Non-limiting embodiments include parenteral administration, such as intradermal, intramuscular, subcutaneous, transcutaneous, mucosal, or oral administration.

The total dose of the composition provided to a subject during one administration can be varied as is known to the skilled practitioner.

It is also possible to provide one or more booster administrations of one or more of the vaccine compositions. If a boosting vaccination is performed, typically, such a boosting vaccination will be administered to the same subject at a moment between one week and 10 years, preferably between two weeks and six months, after administering the composition to the subject for the first time (which is in such cases referred to as “priming vaccination”, for example, for active immunization of RSV naïve infants). Boosting vaccination can also be performed at regular intervals such as for example on yearly basis to maintain optimal protection every RSV season. Such boosting regimen is appropriate for example for adults such as for example adults over 60. In alternative boosting regimens, it is also possible to administer different vectors, e.g., one or more adenovirus, or other vectors such as modified vaccinia virus of Ankara (MVA), or DNA, or protein, to the subject after the priming vaccination. It is, for instance, possible to administer to the subject a recombinant viral vector hereof as a prime, and boosting with an aqueous immunogenic composition as disclosed herein.

In certain embodiments, the administration comprises a priming administration and at least one booster administration. In certain other embodiments, the administration is provided annually. In still other embodiments, the administration is provided annually together with an influenza vaccine.

The vaccines provided by the present disclosure may be used together with one or more other vaccines. For example, in adults they may be used together with an influenza vaccine, Prevnar, tetanus vaccine, diphtheria vaccine, pertussis vaccine or a COVID-19 vaccine. For pediatric use, vaccines provided by the present disclosure may be used with any other vaccine indicated for pediatric patients.

In order that this invention may be better understood, the following examples are set forth. These examples are for the purpose of illustration only and are not to be construed as limiting the scope of the invention in any manner. The following Examples illustrate some embodiments of the invention.

### EXAMPLES

### Example 1—Material and Methods

### a) RSV F Protein

Unless otherwise specified, the RSV F protein(s) used in the Examples are as follows:
RSV F protein of subtype A comprising a polypeptide of SEQ ID NO: 1 and a polypeptide of SEQ ID NO: 2 (RSV A in the below example section).
RSV F protein of subtype A comprising a polypeptide of SEQ ID NO: 3 and a polypeptide of SEQ ID NO: 4 (RSV B in the below example section).

Any RSV F protein could be used in the below examples, including those described in any of WO2017/109629, WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2022/002894 and WO2018/109220.

In particular, any RSV F protein mutant disclosed in WO2017/109629, WO2020026147 and WO2022/002894 can be used in the methods and compositions disclosed therein and in the experiments from the below examples.

### b) Handling of the RSV F Protein Trimer in Prefusion Conformation

Due to potential aggregation and loss of prefusion content, RSV F protein trimers in the prefusion conformation should not be kept at 2-8° C. or on ice. It is recommended for frozen samples larger than 1 mL to be thawed in 25° C. water bath. Thawed samples should be kept at room temperature when performing analysis, and frozen at ≤−40° C. when not in use. It is recommended that samples be flash frozen with liquid nitrogen or ethanol/dry ice slurry prior to storing at ≤−40° C.

### c) Size Exclusion Chromatography (SEC-HPLC).

Percentage of Trimer, HMMS and LMMS were measured by size exclusion chromatography (SEC-HPLC). SEC-HPLC is an analytical method known to the skilled person and used to determine the relative content of high molecular mass species (HMMS), trimer and low molecular mass species (LMMS) in the RSV F protein of subtype A or B samples obtained by the methods of the invention. SEC-HPLC separates molecules by their hydrodynamic volume.

When the analyte is applied to the head of the column bed, molecules that are smaller than the pores of the packing material can diffuse into and out of the pores, whereas those that are larger do not enter the pores. As a result, the larger molecules pass through the column more quickly and smaller molecules more slowly. Once the species elute, they are detected by UV absorption at 280 nm. Low Molecular Mass species (LMMS) is the term used for all species of apparent molecular mass less than the trimer as measured by SEC-HPLC. They elute after the trimer peak. High Molecular Mass species (HMMS) is the term used for all peaks of apparent molecular mass greater than the trimer as measured by SEC-HPLC. They elute before the trimer peak and include aggregates.

### d) Prefusion and Trimer Content by AM14 Fab Titration by SEC

The prefusion conformation content in the RSV F protein trimer was determined through binding with AM14 Fab. AM14 Fab binds to an epitope on RSV F protein that is both trimeric and prefusion specific. RSV F protein trimer in prefusion conformation is reconstituted with purified water and incubated in solution with excess AM14 Fab at two ratios. The remaining free AM14 Fab in each sample is then separated by size-exclusion high performance liquid chromatography (SE-HPLC) and quantitated by UV absorbance at 280 nm against a single point standard curve of AM14 Fab. The AM14 Fab prefusion content of RSV composition is calculated based on the 1:1 (RSV A and/or B monomer: AM14 Fab) binding stoichiometry.

### e) Prefusion and Trimer Content by AM14 Fab Titration by AM14 ELISA

The ELISA measures the dose-dependent binding of the trimeric prefusion specific antibody to prefusion F protein in serially diluted RSV A, RSV B, and composition samples in solution. A microtiter plate (Plate 1) is coated with RSV A, RSV B, and composition reference material, as applicable. In a separate dilution plate (Plate 2), serial dilutions of reference material and samples are co-incubated with the trimeric prefusion specific antibody and a secondary horseradish peroxidase (HRP)-labeled antibody, which binds to the trimeric prefusion specific antibody for signal detection. The trimeric prefusion specific antibody is added in excess, and during this incubation the trimeric prefusion specific antibody binds to any trimeric prefusion F protein present in solution in Plate 2. After incubation, the contents in Plate 2 are transferred to Plate 1 and incubated. During this second incubation, any excess the trimeric prefusion specific antibody not bound to F protein in Plate 2 will bind to the reference material coated on Plate 1. After incubation, Plate 1 is washed to remove any unbound material. Peroxidase substrate is added and the colorimetric response is measured spectrophotometrically. Assessment of prefusion content is based on a comparison between the dose response of the test sample to that of RSV A reference material, RSV B reference material, or composition reference material, as applicable.

### f) Prefusion Content by AM22 Fab Titration by SEC

The prefusion conformation content in the RSV F protein was determined through binding with AM22 Fab. AM22 Fab binds to an epitope on RSV F protein that is prefusion specific. RSV F protein trimer in prefusion conformation is reconstituted with purified water and incubated in solution with excess AM22 Fab at two ratios. The remaining free AM22 Fab in each sample is then separated by size-exclusion high performance liquid chromatography (SE-HPLC) and quantitated by UV absorbance at 280 nm against a single point standard curve of AM22 Fab. The AM22 Fab prefusion content of RSV composition is calculated based on the 1:1 (RSV A and/or B monomer: AM22 Fab) binding stoichiometry.

### Example 2. Effect of pH, Buffer and Excipient on Aggregation and Prefusion Content of an Aqueous Immunogenic Composition Comprising RSV Protein F Trimer of Subtype B in the Prefusion Conformation

The purpose of this study was to evaluate simple formulations to elucidate the effects of buffer choice, excipients, and pH to be used in a composition comprising a RSV F protein trimer in the prefusion conformation. The RSV F protein is a particularly unstable protein due to its propensity to convert to the active postfusion form. The RSV F protein constructs used in this study have been stabilized in the prefusion conformation. Although more stable, these constructs are still considered not completely stabilized. The unstable nature makes it difficult to find a formulation that will retain quality attributes for an extended storage period. These studies were conducted with RSV B as defined in Example 1.

The 18 different formulations used in this study are described in Table 1. Each buffer was prepared by dissolution of the necessary components followed by titration to the appropriate pH. Each formulation was buffer exchanged using Amicon 10 kDa MWCO filter centrifuge tubes for a total buffer flow through greater than 10× to obtain the formulation with the desired composition.

TABLE 1
Formulation Description

| Sample Name | Buffer | pH | Excipients |
|---|---|---|---|
| M01 | 20 mM Tris | 7.4 | N/A |
| M02 | 20 mM Tris | 8.0 | N/A |
| M03 | 20 mM HEPES | 7.4 | N/A |
| M04 | 20 mM Tris | 7.4 | NaCl 50 mM |
| M05 | 20 mM Tris | 8.0 | NaCl 50 mM |
| M06 | 20 mM HEPES | 7.4 | NaCl 50 mM |
| M07 | 20 mM Tris | 7.4 | Sucrose 60 mg/mL |
| M08 | 20 mM Tris | 8.0 | Sucrose 60 mg/mL |
| M09 | 20 mM Tris | 7.4 | Sucrose 120 mg/mL |
| M10 | 20 mM Tris | 8.0 | Sucrose 120 mg/mL |
| M11 | 20 mM Tris | 7.4 | Mannitol 60 mg/mL |
| M12 | 20 mM Tris | 8.0 | Mannitol 60 mg/mL |
| M13 | 20 mM Tris | 7.4 | Glycine 60 mg/mL |
| M14 | 20 mM Tris | 8.0 | Glycine 60 mg/mL |
| M15 | 20 mM Tris | 7.4 | Proline 60 mg/mL |
| M16 | 20 mM Tris | 8.0 | Proline 60 mg/mL |
| M17 | 20 mM Tris | 7.4 | Arginine 60 mg/mL |
| M18 | 20 mM Tris | 8.0 | Arginine 60 mg/mL |

Samples were stored at −20° C., 5° C., and 25° C. for the times listed in Table 2.

TABLE 2
Stability schedule

| | | |
|---|---|---|
| Temperature | 1 Week | 3 Weeks |

| | | | |
|---|---|---|---|
| −20° | C. | NT | X |
| 2-8° | C. | X | X |
| 25° | C. | X | X |

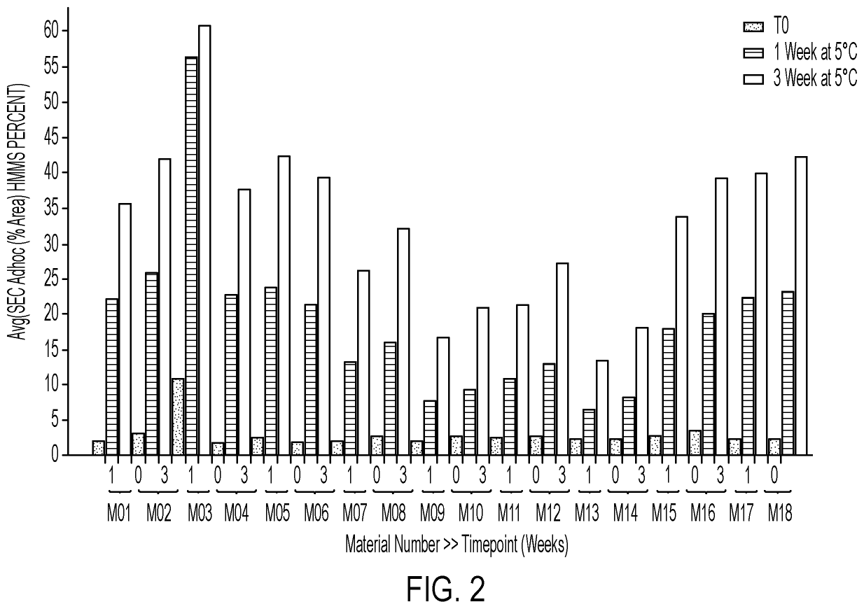

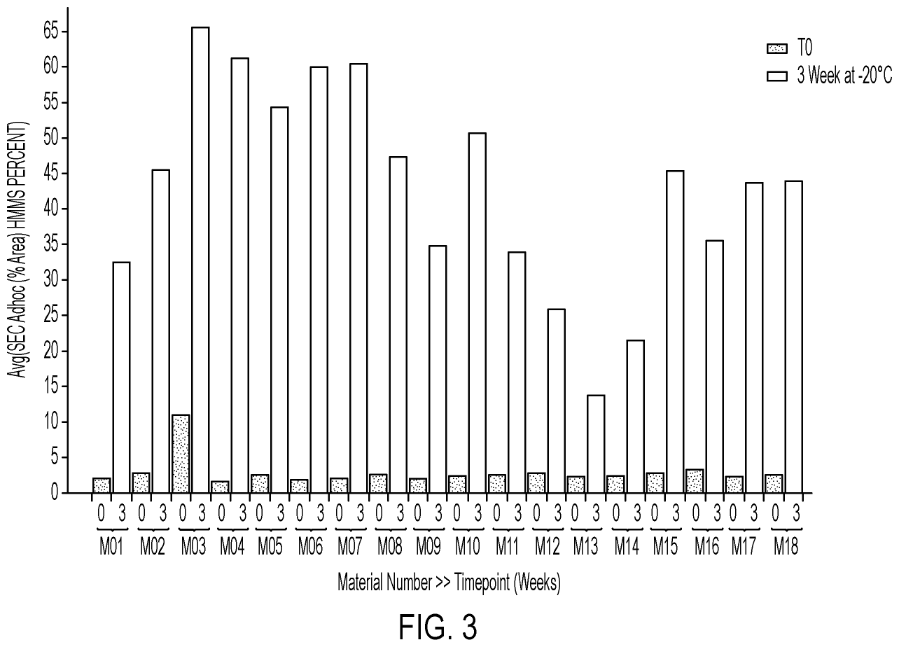

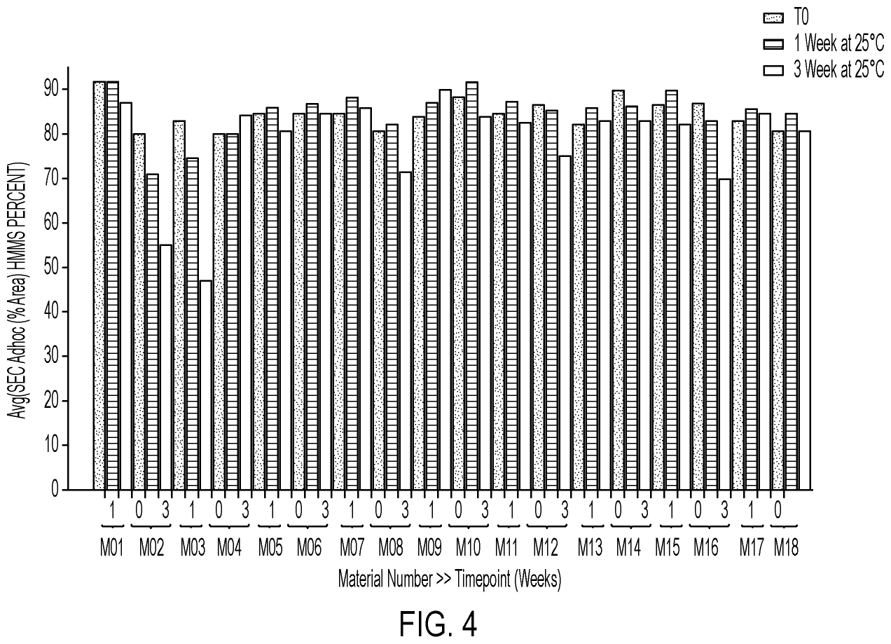

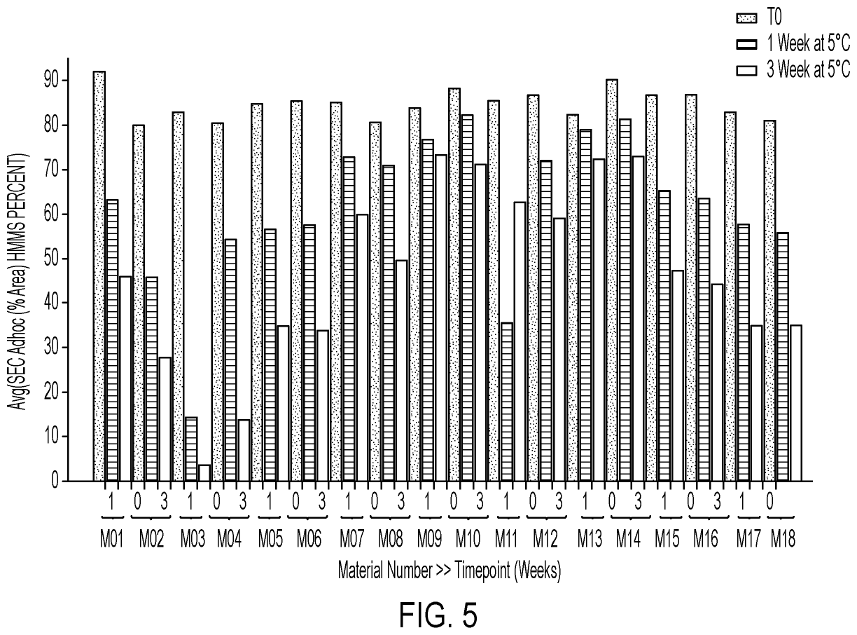

The results from all experiments are shown in FIGS. 1 to 5. The percentage of HMMS was obtained by SEC-HPLC and the prefusion content was obtained by AM22-Fab titration as disclosed in Example 1. The results of this study indicate that Tris buffer provides more stability than HEPES at all conditions in all samples tested. Indeed, the comparison of MO1 (Tris, pH 7.4) and M03 (HEPES, pH 7.4) at all conditions tested shows a lower percentage of HMMS and a higher prefusion content when Tris is used as compared to HEPES. HEPES buffer was tested due to its greater pH stability at lower temperatures, but it did not improve stability at the 5° C. or −20° C. conditions when compared to Tris. However, addition of NaCl to the buffers at 50 mM concentration diminished differences in the buffers ability to preserve quality attributes.

Sodium chloride was shown to preserve prefusion content and to minimize aggregation. Such effect is illustrated for example by the comparison of M03 (HEPES, pH 7.4) and M06 (HEPES, pH 7.4, NaCl) at all conditions tested.

Arginine and proline were tested as potential alternatives to NaCl but did not provide any more stability than NaCl.

Another teaching of this study is the effect of sucrose on aggregation and prefusion content. Increasing amounts of sucrose showed increased retention of prefusion conformation and a smaller increase in % HMMS over the time range tested. Indeed, the comparison of the % HMMS and prefusion content values obtained with conditions M01 and M02 (Tris, pH 7.4 or 8.0, no sucrose) and the values obtained with conditions M07 to M09 (same conditions with sucrose at 60 mg/mL or 120 mg/mL) show a lower % HMMS and a higher prefusion content when sucrose added to the composition.

Temperature is a factor known to affect stability of proteins and, generally, proteins are more stable at low temperature such as 4° C. as compared for example to room temperature (see for example Campa et al, Vaccines 2021, 9, 1114). However, the present study clearly displays that RSV F protein trimers are more stable at 25° C. than at 5° C. For every formulation after three weeks at 25° C. the % HMMS was lower and the prefusion content was higher than after three weeks at 5° C. The study also showed that freezing at −20° C. is a less favorable condition for RSV F protein prefusion stability compared to 25° C. and 5° C. These data illustrate the unique behavior of the stabilized RSV prefusion F protein and challenges in designing an immunogenic aqueous composition comprising such antigen.

The data of FIGS. 1 to 5 demonstrate that the presence of sodium chloride and sucrose are beneficial to the stability of the RSV F protein trimer in the prefusion conformation in an immunogenic composition.

These data also indicate that aqueous compositions as defined herein and in particular in embodiments E1 to E189 should advantageously be stored according to the method of embodiment E193 to E229.

### Example 3. Effect of Sucrose, NaCl and pH on Aggregation and Prefusion Content of an Aqueous Immunogenic Composition Comprising RSV Protein F Trimer of Subtype a in the Prefusion Conformation

This study was performed to evaluate components of compositions comprising RSV F protein trimers in the prefusion conformation for use as a vaccine for the prevention of RSV disease. The antigen in the prefusion conformation, recombinantly produced in CHO cells, elicits greater RSV neutralizing antibody titers than postfusion antigens in experimental animal models. As the stabilization of prefusion F protein is critical, the objective of formulation development was to select excipients and conditions that could maintain the prefusion stability over storage time.

A study was conducted with RSV A to evaluate three primary formulation factors: 1) pH (6.0-8.0); 2) NaCl concentration (0-100 mM); and 3) Sucrose concentration (0-9.0%). The study design is described in Table 3.

TABLE 3
Study Design for Assessing pH, NaCl and Sucrose for a composition
comprising a RSV F protein trimer in the prefusion conformation

| Sample^{a} # | pH | NaCl (mM) | Sucrose (%) |
|---|---|---|---|
| 1 | 8 | 100 | 0 |
| 2 | 8 | 0 | 0 |
| 3 | 8 | 0 | 9 |
| 4 | 6 | 50 | 4.5 |
| 5 | 6 | 100 | 9 |
| 6 | 7 | 0 | 4.5 |
| 7 | 6 | 100 | 0 |
| 8 | 7 | 50 | 0 |
| 9 | 8 | 50 | 4.5 |
| 10 | 7 | 50 | 4.5 |
| 11 | 7 | 50 | 9 |
| 12 | 8 | 100 | 9 |
| 13 | 7 | 100 | 4.5 |
| 14 | 7 | 50 | 4.5 |
| 15 | 6 | 0 | 9 |
| 16 | 6 | 0 | 0 |
| 17^{b} | 7 | 50 | 0 (+4.5% |
| | | | Trehalose) |
| 18^{c} | 7 | 50 | 3 (+3% |
| | | | mannitol) |
| 19^{c} | 7 | 50 | 0 (+3% Trehalose +3% |
| | | | mannitol) |

^{a}All samples contained 20 mM His-Tris buffer and 0.02% PS 80
^{b}For comparison of sucrose vs trehalose
^{c}For assessment of mannitol's effect on stability

A total of 16 formulations were prepared to assess the effect of pH, NaCl and sucrose in a composition comprising an RSV F protein trimer in the prefusion conformation. An additional three formulations were included to compare the choice of the cryoprotectant species (sucrose vs. trehalose) and to assess the effect of mannitol on the antigen stability. Samples were prepared at 0.24 mg/mL RSV A in a matrix of 20 mM His-Tris buffer with 0.02% PS-80. A mixture of the His-Tris buffer systems was used as it gave the ability to control pH well in the range of 6.0-8.0. PS-80 was added to mitigate any potential protein adsorption onto the container. The samples were held at 5° C. and 25° C. for two weeks and monitored for prefusion content by AM14 ELISA, % aggregation by SEC-HPLC, protein concentration by UV280, pH and appearance. No differences were observed in protein concentration, pH and appearance. AM14 prefusion content and aggregation results are summarized in Table 4.

TABLE 4
Study Results for Formulations Held at 5° C. and 25° C. for 2 Weeks

| Formulations | | | | | | | | | | | | | |
|---|---|---|---|---|---|---|---|---|---|---|---|---|---|
| (0.24 mg/mL RSVA) | | | | AM14 prefusion content (%)^{a} | | | | | Aggregates (%) | | | | |
| | | NaCl | Sucrose | | 1 wk | | 2 wk | | | 1 wk | | 2 wk | |
| | pH | (mM) | (%) | T0 | 5° C. | 25° C. | 5° C. | 25° C. | T0 | 5° C. | 25° C. | 5° C. | 25° C. |
| F1 | 8 | 100 | 0 | 120 | 96 | 112 | 100 | 93 | 5 | 5 | 9 | 5 | 10 |
| F2 | 8 | 0 | 0 | 86 | 86 | 58 | 75 | 56 | 5 | 7 | 15 | 9 | 23 |
| F3 | 8 | 0 | 9 | 95 | 104 | 81 | 70 | 72 | 6 | 6 | 12 | 13 | 18 |
| F4 | 6 | 50 | 4.5 | 57 | 76 | 59 | 62 | 86 | 5 | 5 | 6 | 9 | 10 |
| F5 | 6 | 100 | 9.0 | 91 | 78 | 97 | 76 | 98 | 5 | 5 | 9 | 7 | 11 |
| F6 | 7 | 0 | 4.5 | 99 | 101 | 102 | 77 | 83 | 6 | 6 | 9 | 11 | 13 |
| F7 | 6 | 100 | 0 | 88 | 61 | 75 | 38 | 55 | 7 | 7 | 9 | 8 | 10 |
| F8 | 7 | 50 | 0 | 100 | 99 | 86 | 101 | 85 | 6 | 6 | 15 | 7 | 10 |
| F9 | 8 | 50 | 4.5 | 94 | 94 | 84 | 92 | 60 | 6 | 6 | 11 | 6 | 10 |
| F10 | 7 | 50 | 4.5 | 104 | 93 | 82 | 71 | 81 | 5 | 7 | 10 | 8 | 9 |
| F11 | 7 | 50 | 9.0 | 102 | 101 | 91 | 90 | 82 | 5 | 5 | 8 | 7 | 9 |
| F12 | 8 | 100 | 9.0 | 100 | 98 | 82 | 72 | 55 | 6 | 5 | 9 | 7 | 10 |
| F13 | 7 | 100 | 4.5 | 97 | 76 | 90 | 75 | 37 | 5 | 5 | 9 | 7 | 8 |
| F14 | 7 | 50 | 4.5 | 102 | 85 | 77 | 78 | 78 | 5 | 6 | 7 | 9 | 10 |
| F15 | 6 | 0 | 9 | 106 | 86 | 99 | 84 | 91 | 6 | 11 | 14 | 18 | 15 |
| F16 | 6 | 0 | 0 | 80 | 65 | 87 | 67 | 76 | 6 | 11 | 12 | 25 | 19 |
| F17 | 7 | 50 | 4.5 Trh | 86 | 59 | 105 | 64 | 64 | 5 | 6 | 7 | 7 | 8 |
| F18 | 7 | 50 | 3 Suc + | 91 | 56 | 83 | 85 | 78 | 5 | 10 | 12 | 8 | 9 |
| | | | 3 Mtl | | | | | | | | | | |
| F19 | 7 | 50 | 3 Trh + | 93 | 74 | 75 | 75 | 84 | 5 | 6 | 9 | 6 | 8 |
| | | | 3 Mtl | | | | | | | | | | |

^{a}Relative to standard

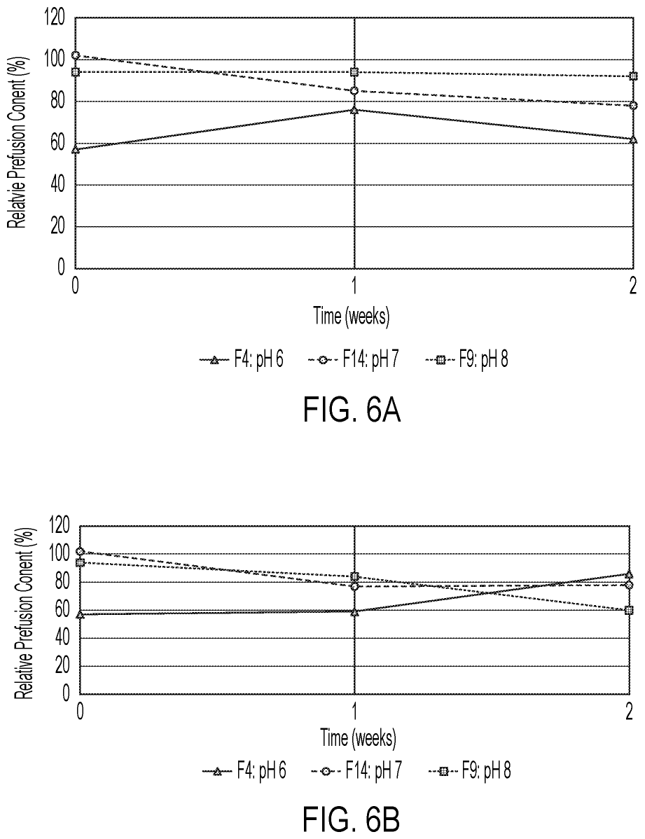

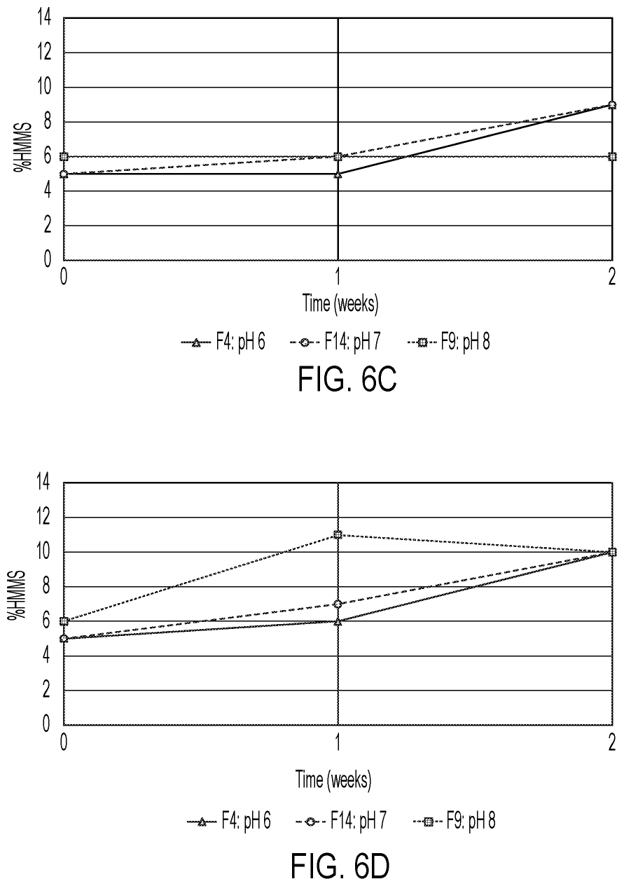

Some of the results of this experiment are also summarized in FIGS. 6A to 6D, 7A to 7D and 8A to 8D.

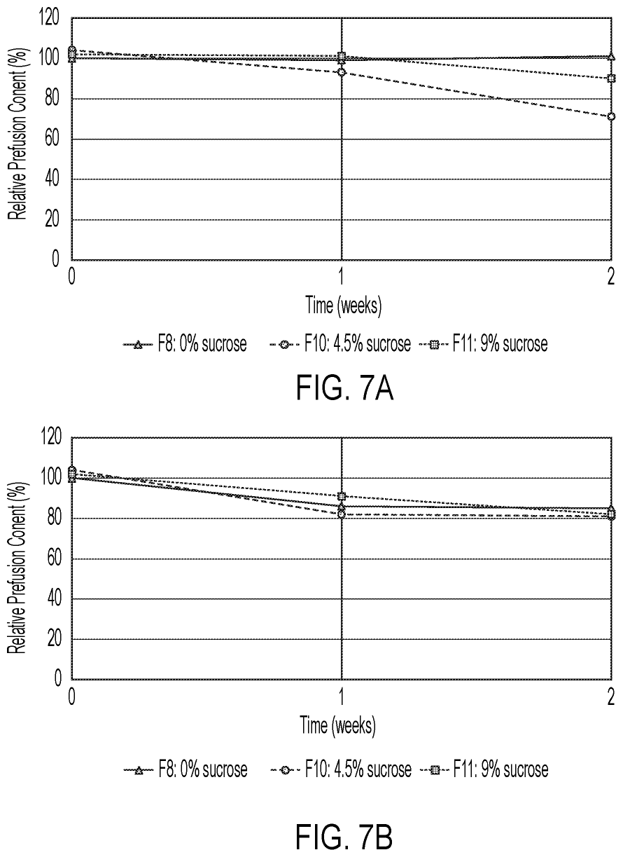

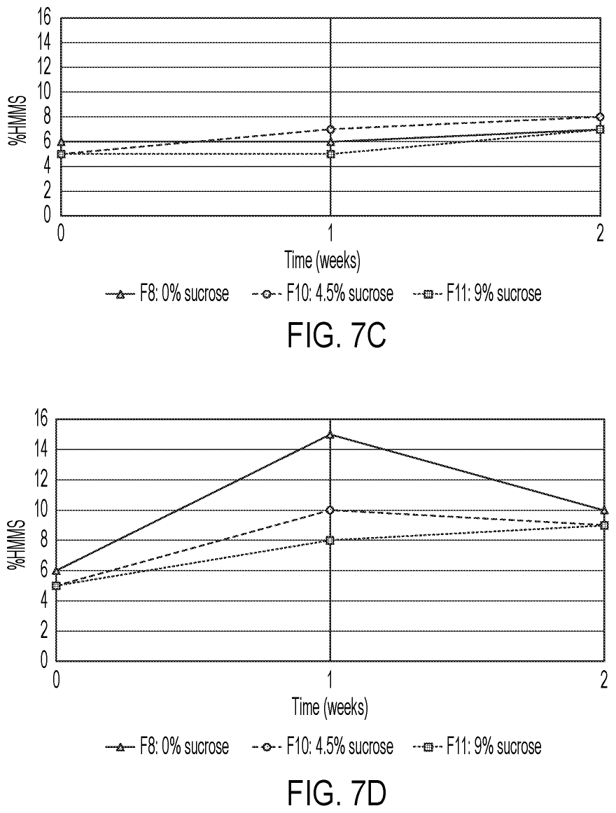

When comparing formulations at pH 7 with 50 mM NaCl and varying concentrations of sucrose (Sample F8, F11 and F11 in Table 4), no significant difference in aggregation is observed after 2 weeks of storage at both temperatures (see FIGS. 7C and 7D). A direct comparison of sucrose and trehalose formulations (Sample F10 and F17 in Table 4) suggested that they had a similar effect on maintaining the prefusion stability. Therefore, trehalose was not further considered, and sucrose was included in the formulation as a cryoprotectant in particular to maximize stability storage of the liquid formulation in the frozen state prior to lyophilization.

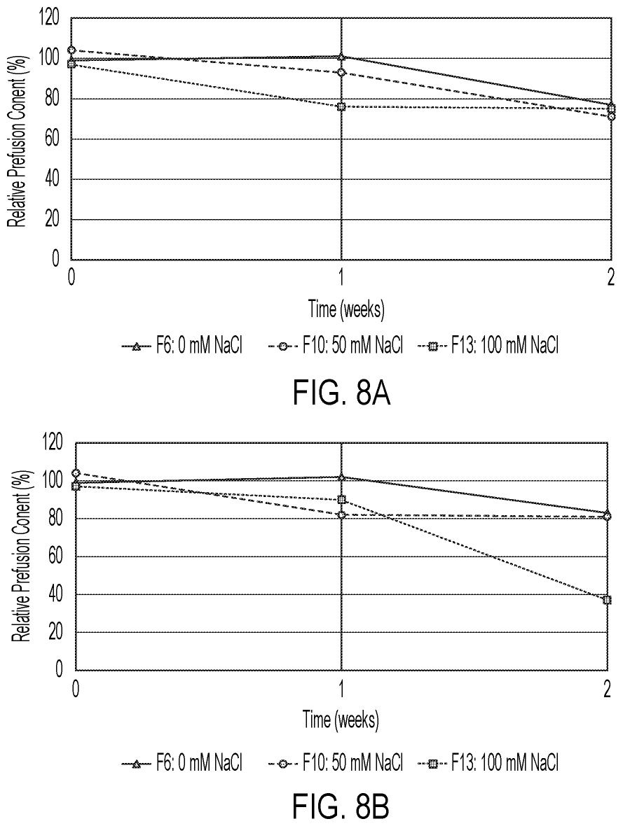

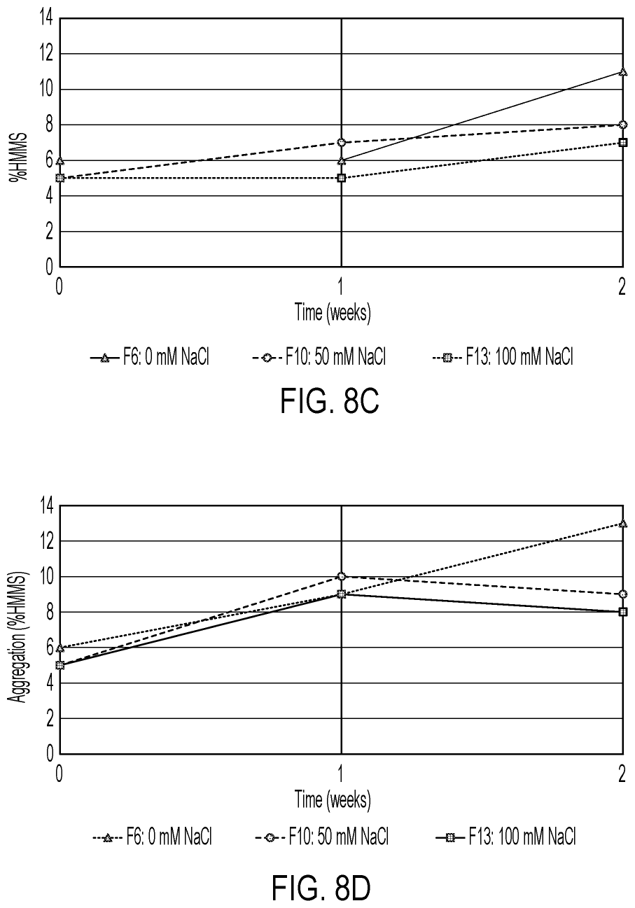

Also, NaCl was shown to be highly beneficial with respect to controlling RSV F protein aggregation (see FIGS. 8C and 8D).

### Example 4—Effect of Alternative Excipient and Concentration Levels on Aggregation of RSV F Protein of Subtype B

In addition to the formulation parameters evaluated in the design of experiment (DoE) study, additional conditions were explored to investigate whether the stability of the protein antigen could be further improved. Excipients from different classes were selected with the aim to further improve the composition. Samples containing 0.5 mg/mL RSV B were prepared in the presence of different excipients (Table 5) and held at 5° C. and 25° C. for one week. A preferred formulation derived from the data of Example 3 (20 mM Tris, 50 mM NaCl, 4% sucrose, 0.02% PS 80 at pH 7.4) was used as a control. The sample aggregation levels at initial and after 3 and 7 days were evaluated using SEC. Samples that exhibited high aggregation levels at initial were excluded from further testing. The results are shown in below Table 5 and suggest the following:
1) the RSV F protein samples stored at 5° C. were more susceptible to aggregation than at 25° C.; This confirms the unique behavior of RSV F protein trimer in the prefusion conformation already observed in the experiments of Example 2.
2) excipients/conditions explored offered no significant improvement when compared to the composition components identified in Examples 2 and 3.
3) various amounts of sucrose and sodium chloride can be used to reduce the percentage of aggregates as well as the impact of pH variation on aggregation.

TABLE 5
Aggregation Results for Additional Excipients Screening

| | Formulation composition (0.5 mg/mL RSV F | | | | | | | | | | |
|---|---|---|---|---|---|---|---|---|---|---|---|
| | protein) | | | | | | % Aggregates | | | | |
| | | NaCl | Tris | PS 80 | Sucrose | | | 3 Days | | 7 Days | |
| Sample | pH | (mM) | (mM) | (%) | (%) | Other | T0 | 5° C. | 25° C. | 5° C. | 25° C. |
| A | 7.4 | 50 | 20 | 0.02 | 4 | 25 mM | 82^{a} | Discontinued | | | |
| | | | | | | Glutamic | | | | | |
| B | 7.4 | 50 | 20 | 0.02 | 4 | 100 mM Arg | 15 | 12 | 12 | 21 | 11 |
| C | 7.4 | 50 | 20 | 0.02 | 4 | 50 mM Arg + | 78^{a} | Discontinued | | | |
| | | | | | | 25 mM Glu | | | | | |
| D | 7.4 | 50 | 20 | 0.02 | 9 | NA | 13 | 11 | 11 | 18 | 11 |
| E | 7.4 | 50 | 20 | 0.02 | 4 | 0.5 mg/L | 16 | Discontinued | | | |
| | | | | | | CuSO4 | | | | | |
| F | 7.4 | 50 | 20 | 0.4 | 4 | NA | 12 | 14 | 13 | 25 | 11 |
| G | 8.5 | 50 | 20 | 0.02 | 4 | NA | 14 | 13 | 13 | 26 | 14 |
| H | 6.2 | 50 | 20 | 0.02 | 4 | 20 mM His | 21 | 14 | 34 | Discontinued | |
| I | 7.4 | 200 | 20 | 0.02 | 4 | NA | 15 | 13 | 13 | 24 | 12 |
| Ctl | 7.4 | 50 | 20 | 0.02 | 4 | NA | 15 | 14 | 13 | 24 | 11 |

^{a}pH spike was noticed at Glu addition, which may result in the high aggregation

### Example 5. Stress Studies to Assess Robustness of a Composition Comprising RSV F Protein of Subtype A and B, Sucrose and Sodium Chloride

To ensure the aqueous immunogenic compositions comprising a RSV F protein trimer in prefusion conformation are able to sustain the freezing/thawing (F/T) and other stresses to be encountered during processing and handling, the stability of both RSV F protein of subtype A and B in a composition according to the invention (20 mM Tris, 50 mM NaCl, 4% sucrose and 0.02% PS 80 at pH 7.4) were assessed in the following stress studies:
1. Three to five−70° C. to RT freezing and thawing cycles;
2. 24 hour agitation (500 rpm) at ambient RT;
3. One week liquid stability at 5° C. and 25° C.

Samples were held in polypropylene tubes for RSV A F/T study (1 mL fill in 3 mL tubes), in Type 1 glass vials for RSV B F/T study, as well as agitation and 1 week liquid stability studies of RSVA and RSVB (0.5 mL fill in 2 mL vials). Samples were monitored with respect to AM14 prefusion content (using the ELISA assay disclosed in example 1), aggregation (SEC HPLC, see Example 1) and total protein concentration.

The results (Table 6) demonstrated that RSV A was stable under all conditions tested. For RSV B, AM 14 prefusion was stable upon one week storage at 25° C. with a 5% increase in aggregation. At 5° C., RSV B stability was reduced compared to 25° C. and showed an 11% increase in aggregation. RSV B was stable for up to five F/T cycles. Upon agitation RSV B showed a slight decrease in the AM14 prefusion content compared to control, which is acceptable in view of the assay variablity.

TABLE 6
Stability of RSV A and RSV B Under Stress Conditions

| | # of Freeze | | Agitation | | | | |
|---|---|---|---|---|---|---|---|
| | thaw Cycles | | (500 rpm at RT) | | | | |
| | (−80° C./RT) | | Static | | 1-Week Liquid Stability | | |
| RSV A | 1X | 3X | Control | 24 hrs | T0 | 5° C. | 25° C. |
| AM14 Prefusion (%) | 84 | 83 | 83 | 84 | 88 | 87 | 97 |
| % Aggregates | 9 | 9 | 4 | 4 | 7 | 9 | 11 |
| Concentration (mg/mL) | 1.1 | 1.0 | 1.03 | 1.02 | 0.49 | 0.50 | 0.50 |
| by UV280 | | | | | | | |

| | | | Static | | | | |
|---|---|---|---|---|---|---|---|
| RSV B | 1X | 5X | Control | 24 hrs | T0 | 5° C. | 25° C. |
| AM14 Prefusion (%) | 70 | 77 | 77 | 66 | 77 | 67 | 72 |
| % Aggregates | 12 | 13 | 13 | 14 | 9 | 20 | 14 |
| Concentration (mg/mL) | 1.24 | 1.21 | 1.22 | 1.23 | 1.24 | 1.22 | 1.21 |
| by UV280 | | | | | | | |

### Example 6. Lyophilization of Aqueous Compositions Comprising Sucrose, Sodium Chloride and a RSV F Protein Trimer in Prefusion Conformation

Compositions comprising RSV A were made and assessed at two concentration levels: 480 μg/mL and 120 μg/mL in 2 different matrices: 1) 9% sucrose and 2) 2% sucrose and 4% mannitol, both in 20 mM Tris, 50 mM NaCl, 0.02% PS 80 at pH 7.4. Samples were lyophilized using SP Scientific LyoStar 3® using conditions disclosed in Table 7 and Table 8. Stability, with respect to AM14 prefusion content, aggregation, protein concentration, pH and moisture, was monitored pre- to post-lyophilization and short-term at 5° C. and 25° C. storage. All samples had acceptable cake appearance post lyophilization with moisture below 0.5%. No changes in pH and protein concentration were observed post reconstitution (data not shown). AM14 prefusion content and aggregation (SEC HPLC-see Example 1) results demonstrated that a lyophilized formulation was feasible for the aqueous composition comprising RSV prefusion F protein of subtype A disclosed herein. The AM14 prefusion content was maintained after lyophilization and remained stable for the short term it was assessed. The increase in aggregation was within 10% (Tables 9 and 10).

TABLE 7
Lyophilization Cycle Used for Samples
Containing 9% Sucrose (96 hours Cycle)

| | Temp | Rate | Hold | Pressure |
|---|---|---|---|---|
| Step | (° C.) | (° C./min) | (min) | (mTorr) |
| Freezing | −50 | 0.5 | 120 | N/A |
| Annealing | −10 | 0.5 | 300 | N/A |
| Re-Freezing | −50 | 0.5 | 120 | N/A |
| Extra Freeze/ | −50 | N/A | 10 | 25 |
| Vacuum Release | | | | |
| Primary Drying | −35 | 0.5 | 3600 | 25 |
| Secondary Drying | 40 | 0.25 | 900 | 25 |
| Storage | 5 | 0.5 | N/A | 25 |

TABLE 8
Lyophilization Cycle Used for Samples Containing
2% Sucrose + 4% Mannitol (55 hrs Cycle)

| | Temp | Rate | Hold | Pressure |
|---|---|---|---|---|
| Step | (° C.) | (° C./min) | (min) | (mTorr) |
| Freezing | −50 | 0.5 | 120 | N/A |
| Annealing | −10 | 0.5 | 300 | N/A |
| Re-Freezing | −50 | 0.5 | 180 | N/A |
| Extra Freeze/Vacuum | −50 | N/A | 30 | 100 |
| Release | | | | |
| Primary Drying | −5 | 0.2 | 1440 | 100 |
| Pre-secondary Drying | 5 | 0.2 | 5 | 100 |
| Secondary Drying | 40 | 0.5 | 600 | 100 |
| Storage | 5 | 0.5 | N/A | 100 |

TABLE 9
Short term Stability of Lyophilized
RSV A Monovalent in 9% Sucrose Matrix

| | % AM 14 Prefusion Content | | | | % Aggregate | | | |
|---|---|---|---|---|---|---|---|---|
| | | Post | | | | Post | | |
| RSV A. | | Lyo | 1 Month | | | Lyo | 1 Month | |
| (μg/mL) | Pre-Lyo | T0 | RT | 5° C. | Pre-Lyo | T0 | RT | 5° C. |
| 480 | 79 | 78 | 76 | 73 | 3 | 4 | 7 | 8 |
| 480 | 88 | 82 | 96 | 79 | 7 | 8 | 8 | 9 |
| 120 | 81 | 77 | 75 | 76 | 3 | 3 | 5 | 7 |
| | (rpt^{a} 75, 76) | | | | | | | |
| 120 | 93 | 86 | 78 | 82 | 7 | 10 | 11 | 11 |

^{a}Repeated results

TABLE 10
Short term Stability of Lyophilized RSV A Monovalent in 2% Sucrose and 4% Mannitol Matrix

| | % AM 14 Prefusion Content | | | | | | | % Aggregate | | | | | | |
|---|---|---|---|---|---|---|---|---|---|---|---|---|---|---|
| | | | | | 5° C. | | | | Post | | | 5° C. | | |
| RSV A | Pre- | Post | RT | | | | 2.5 | Pre- | Lyo | RT | | | | 2.5 |
| (μg/mL) | Lyo | Lyo T0 | 1 m | 2 m | 1 m | 2 m | m | Lyo | T0 | 1 m | 2 m | 1 m | 2 m | m |
| 480 | 71 | 73 | 65 | NT | 68 | NT | 65 | 4 | 4 | 6 | NT | 7 | NT | 7 |
| 480 | 71 | 71 | 81 | 68 | 78 | 61 | NT | 7 | 8 | 7 | 8 | 7 | 11 | NT |
| 480 | 67 | 72 | 65 | NT | 61 | 59 | NT | 4 | 6 | 7 | NT | 5 | 7 | NT |
| 120 | 72 | 68 | 63 | NT | 57 | NT | 54 | 3 | 4 | 6 | NT | 7 | NA | 6 |
| 120 | 68 | 78 | 58 | 65 | 65 | 61 | NT | 8 | 8 | 9 | 7 | 8 | 7 | NT |
| 120 | 65 | 62 | 61 | NT | 46 | 55 | NT | 6 | 6 | 7 | NT | 8 | 6 | NT |

NT: Not tested

Lyophilization feasibility was also assessed for aqueous compositions comprising RSV B prefusion F alone (480 μg/mL) or RSV A and RSV B (480 μg/mL total, 240 μg/mL each) in 20 mM Tris, 50 mM NaCl, 0.02% PS 80, 2% sucrose and 4% mannitol at pH 7.4. Samples were monitored for AM14 prefusion content and % aggregation (SEC HPLC-see Example 1) from pre- to post-lyophilization and after 2 months storage at 5° C. Preliminary data (Table 11) 10 suggested that RSV B was viable to be lyophilized as well. Increased aggregation was observed to a greater extent with RSV B monovalent than in the RSV A and RSV B bivalent composition.

TABLE 11
Short term Stability of Lyophilized RSV B Monovalent and RSV
A + RSV B Bivalent in 2% Sucrose and 4% Mannitol Matrix

| | AM14 Prefusion Content^{a} (%) | | | % Aggregates by SEC | | |
|---|---|---|---|---|---|---|
| | | Postlyo | | | Postlyo | |
| | | | 2 | | | 2 |
| Sample | Pre-lyo | T0 | months | Pre-lyo | T0 | months |
| RSV B | 90 | 82 | 101 | 9 | 16 | 24 |
| RSV A + RSV B | 100 | 81 | 116 | 9 | 12 | 11 |

^{a}Relative to standard

The above data show that the aqueous immunogenic compositions comprising RSV protein F trimer in the prefusion conformation of subtype A and/or B disclosed herein can be lyophilized. For example, a composition comprising 9% sucrose could be lyophilized in 96 hours while maintaining prefusion content and limiting the increase in aggregation. Another composition comprising sucrose and mannitol could be lyophilized in 55 hours while maintaining acceptable prefusion content and level of aggregation. It is advantageous to reduce the lyophilization time for production purposes, in particular for large scale production.

As demonstrated in Examples 2 to 5, aqueous compositions comprising a RSV F protein trimer in prefusion conformation and sucrose and sodium chloride are advantageous from a stability perspective, in particular regarding the prefusion content and the percentage of aggregation. The data of Example 6 suggest that these compositions can be even further optimized by including another sugar such as mannitol so that the composition can be lyophilized in short period of time without compromising the stability of the product.

### Example 7—Optimization of Aqueous Compositions Comprising RSV F Protein Trimer in Prefusion Conformation to Reduce Duration of Lyophilization while Limiting Loss of Prefusion Content and Increase of Aggregates

To optimize the choice and ratio of stabilizer/bulking agents to be used in the composition, 10 different matrices shown in Table 12 were selected and lyophilized. Developmental lyophilization cycles were applied to produce lyophilized compositions containing sucrose/mannitol or sucrose/glycine. The original lyophilization cycle disclosed in Table 7 (˜96 hours) was used to produce the control in this study. All test formulations were monitored for aggregation (% HMMS), and prefusion content by AM14 ELISA and AM22 Fab titration before and after lyophilization using the assays disclosed in Example 1.

TABLE 12
RSV F protein composition

| | Common | Sucrose | Mannitol | Glycine | NaCl |
|---|---|---|---|---|---|
| Formulation # | components | %(w/v) | %(w/v) | %(w/v) | (mM) |
| 1 | 240 μg/mL RSV | 3 | 6 | 0 | 50 |
| 2 | A and 240 | 3 | 6 | 0 | 0 |
| 3 | μg/mL RSV B, | 2 | 5.5 | 0 | 50 |
| 4 | 20 mM Tris at | 2 | 5.5 | 0 | 0 |
| 5 | pH 7.4, 0.02% | 1.5 | 0 | 3 | 25 |
| 6 | PS80 | 1.5 | 0 | 3 | 0 |
| 7 | | 1 | 0 | 2.5 | 25 |
| 8 | | 1 | 0 | 2.5 | 0 |
| 9 | | 2 | 4 | 0 | 50 |
| 10 (Gen 1 | | 9 | 0 | 0 | 50 |
| matrix, control) | | | | | |

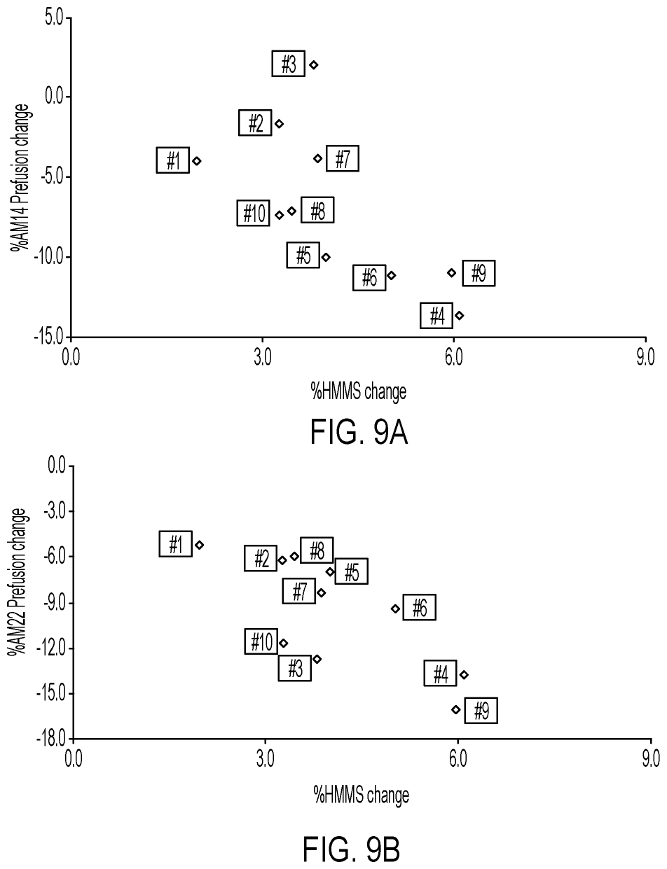

The pre- and post-lyophilization stability results are shown in FIG. 9. The change in aggregation (% HMMS) versus the change in % AM14 prefusion content were plotted in FIG. 9A. Similarly, the change in aggregation (% HMMS) versus the change in % AM22 prefusion content were plotted in FIG. 9B.

Compositions #1 to #9 could all be lyophilized in about 24 hours. For the compositions containing sucrose and mannitol, two compositions containing 3% sucrose/6% mannitol with or without NaCl (#1 and #2 in Table 12) demonstrated the best overall stability among all tested compositions upon lyophilization. Composition #4 (2% sucrose/5.5% mannitol) and composition #9 (2% sucrose/4% mannitol with NaCl) showed the largest drop in prefusion content and the largest increase in aggregation upon lyophilization indicating non-ideal sucrose/mannitol levels and/or ratios. In addition, data in FIG. 9 suggest that the presence of NaCl in the composition can potentially reduce the rate of aggregation in most composition upon lyophilization.

For composition containing sucrose and glycine, the one containing 1% sucrose/2.5% glycine with or without NaCl (#7 and #8 in Table 12) showed marginally better stability than the one containing 1.5% sucrose/3% glycine with or without NaCl (#5 and #6 in Table 12).

In view of the above, inclusion of sucrose and mannitol or sucrose and glycine in composition comprising RSV F protein trimer in the prefusion conformation is particularly advantageous to obtain compositions which can be lyophilized and where the loss of prefusion content and the increase of aggregation is limited. In particular, compositions where the ratio of sucrose to mannitol or sucrose to glycine is between 1 to 1 and 1 to 4, preferably 1 to 2 to 1 to 4, more preferably 1 to 2 provide compositions that can be lyophilized in 24H and where the loss of prefusion content and the increase of aggregation is acceptable.

### Example 8—Development of a Lyophilization Process for RSV F Protein Trimer in the Prefusion Conformation

The objective of the lyophilization development was to develop a target lyophilization cycle by optimizing the conditions for each stage of the lyophilization cycle including freezing, annealing, and drying to produce a visually elegant cake with acceptable product quality. After initial development to obtain the target lyophilization cycle, the robustness of the lyophilization cycle was assessed by challenging the target cycle parameters and evaluating the impact to product quality. The critical quality attributes evaluated during lyophilization process development included residual moisture content, HMMS and prefusion content. Additionally, lyophilization of a mannitol-sucrose formulation can result in the presence of multiple polymorphic forms of mannitol, therefore presence of Mannitol Hemihydrate (MHH) was also monitored, as this phase can have a negative impact on product stability over time.

The major steps in the lyophilization process include the freezing, annealing, drying via sublimation, and drying via desorption of residual moisture. Several parameters must be considered throughout each of the steps including shelf temperature, ramp and hold times, chamber pressure during drying, and product temperature throughout the cycle. Freezing hold temperatures affect the structural characteristics of the frozen matrix. Inclusion of an annealing step reduces vial to vial heterogeneity typically induced during freezing by promoting crystallization and homogeneity of ice crystal size, and maximizing the crystallization of mannitol, which can ultimately minimize differences in cake appearance and reduce drying time. The cycle time, shelf temperature and chamber pressure used during the first ramp of drying directly affect the temperature of the frozen matrix, the rate of sublimation and the structure and appearance of the dried cake. The overall cycle time and shelf temperature utilized during the second ramp of drying primarily impacts the residual moisture level in the dried cake. Selection of the appropriate parameter values for each step collectively enables a lyophilization cycle that produces a dried cake in vial having a desirable appearance of an elegant cake with a low level of residual moisture to support the stabilization of the drug product.

Following thermal analysis of the drug product formulation, initial parameters for the lab-scale lyophilization cycle were selected based on historical process development experience with semi-crystalline formulations (see Table 13 below).

TABLE 13
Target Lyophilization Cycle

| | | Ramp | | | Chamber |
|---|---|---|---|---|---|
| | | Rate | Ramp | Hold | Pressure |
| | Temperature | (° C./ | Time | Time | (mTorr/ |
| Segment | (° C.) | min) | (min) | (min) | μbar) |
| Freezing | −50 | 1.0 | 70 | 60 | NA |
| Annealing | −10 | 0.5 | 80 | 210 | NA |
| Refreezing | −50 | 0.5 | 80 | 60 | NA |
| Vacuum Initiation | −50 | NA | NA | NA | 200 mTorr |
| | | | | | (267 μbar) |
| First Dry Step | 0 | 0.5 | 100 | 0 | 200 mTorr |
| | | | | | (267 μbar) |
| Second Dry Step | 50 | 0.2 | 250 | 515 | 200 mTorr |
| | | | | | (267 μbar) |
| Stoppering | NA | NA | NA | NA | 600,000 mTorr |
| | | | | | (800 mbar) |

Abbreviations:
NA = not applicable

Aggressive cycle conditions were then created to confirm the critical process parameters and define the upper and lower boundaries to be challenged to assess process robustness. The RSV

F protein composition (see Table 14) was lyophilized using the five aggressive cycles described in Table 15 to confirm the critical process parameters and define the upper and lower boundaries to be challenged in the process robustness studies. The cycles assessed the lyophilization parameters of freezing ramp rate, annealing ramp rate and temperature, and drying ramp rate and temperature by challenging the parameter values of the target lyophilization cycle. The cumulative aggressive cycle combined the slow freezing and annealing ramp rates with a high freezing temperature, annealing temperature and drying temperature. Individual cycles were executed as listed in Table 15 and varied either the freezing ramp rate, annealing temperature, annealing ramp rate, or drying temperature and ramp rate while keeping all other parameters consistent with the target cycle values.

TABLE 14
RSV A and B composition pre- and post- lyophilisation

| | RSV F protein | Composition Post- |
|---|---|---|
| | composition to | Reconstitution |
| | be lyophilised | with Sterile Water |
| Ingredient | (mg/mL or g/L) | Diluent (mg/mL) |
| RSV A | 0.32 | 0.24 |
| RSV B | 0.32 | 0.24 |
| Tromethamine^{b} | 0.30^{d} | 0.23^{e} |
| Tris-HCl^{c} | 2.76^{d} | 2.07^{e} |
| Sucrose | 30 | 22.5 |
| Mannitol | 60 | 45.0 |
| Polysorbate 80 | 0.20 | 0.15 |
| NaCl | 2.92^{f} | 2.19^{g} |
| Water for Injection | q.s to 1 mL or L | q.s to 1 mL |

^{b}Also known as Trometamol and Tris Base
^{c}Also known as Tris (hydroxymethyl) aminomethane hydrochloride (Tris-HCl), Tromethamine HCl, and Trometamol HCl
^{d}Tromethamine + Tris-Hydrochloride composition is equivalent to 20 mM Tris
^{e}Tromethamine + Tris-Hydrochloride composition is equivalent to 15 mM Tris
^{f}Equivalent to 50 mM NaCl
^{g}Equivalent to 37.5 mM NaCl

TABLE 15
Critical Process Parameter Assessment Cycles

| | | Freezing | | Annealing | | 2nd |
|---|---|---|---|---|---|---|
| | Freezing | Ramp | Annealing | Ramp | Drying | Drying |
| Cycle | Temp. | Rate | Temp | Rate | Ramp | Temp. |
| Description | (° C.) | (° C./min) | (° C.) | (° C./min) | Rate (° C./min) | (° C.) |
| Target | −50 | 1 | −10 | 0.5 | 0.5 (1^{st} Drying); | 50 |
| | | | | | 0.2 (2^{nd} Drying) | |
| Cumulative | −45 | 0.25 | −5 | 0.2 | 0.1 (1^{st} Drying); | 55 |
| Aggressive | | | | | 0.1 (2^{nd} Drying) | |
| Freezing | −50 | 0.25 | −10 | 0.5 | 0.5 (1^{st} Drying); | 50 |
| Ramp | | | | | 0.2 (2^{nd} Drying) | |
| Aggressive | | | | | | |
| Annealing | −50 | 1 | −5 | 0.5 | 0.5 (1^{st} Drying); | 50 |
| Temp | | | | | 0.2 (2^{nd} Drying) | |
| Aggressive | | | | | | |
| Annealing | −50 | 1 | −10 | 0.2 | 0.5 (1^{st} Drying); | 50 |
| Ramp | | | | | 0.2 (2^{nd} Drying) | |
| Aggressive | | | | | | |
| Drying | −50 | 1 | −10 | 0.5 | 0.5 (1^{st} Drying); | 55 |
| Temp + Ramp | | | | | 0.1 (2^{nd} Drying) | |
| Aggressive | | | | | | |

All experiments were conducted with a LyoStar3 Lyophilizer® from FTS System Inc. The aggressive cycles assessed the impact of freezing ramp rate, annealing ramp rate and temperature, and drying ramp rate and temperature on moisture content, prefusion content, HMMS and presence of MHH in comparison to the lyophilized composition produced using the target cycle. The results are summarized in Table 16.

The impact of the cumulative aggressive cycle to the critical quality attributes indicated a minor impact while HMMS is within typical method variability for SE-HPLC, a drop in prefusion content relative to the pre-lyophilized composition and the lyophilized composition obtained using the target cycle is observed.

To identify the critical parameters that contributed to the observed results of the cumulative aggressive cycle, an evaluation of the results from the cycles that individually varied the parameters for freezing ramp rate, annealing temperature, annealing ramp rate, and the drying temp and ramp was performed. The results indicate freezing ramp rate had the largest impact to prefusion content and was similar to the value observed for the cumulative aggressive cycle. These results suggest the freezing ramp rate is the parameter that most significantly impacts the prefusion content of the lyophilized composition as the prefusion content for the annealing temperature, annealing ramp rate, and drying parameters aggressive cycles are within method variability compared to the lyophilized composition using the target cycle.

Regarding moisture content and presence of MHH, the target cycle, cumulative aggressive cycle, annealing temperature and freezing ramp aggressive cycles were evaluated, as these cycles had the highest risk for moisture content and MHH presence. All cycles produced a lyophilized product with low moisture content at 0.2% to 0.4% by weight and no MHH was evident by Powder X-Ray Diffraction.

TABLE 16
Results: Critical Process Parameter Assessment

| | | | | SE-HPLC | |
|---|---|---|---|---|---|
| | Protein | Residual | Prefusion | % Trimer | |
| | Conc. | Moisture | Content | % HMMS | MHH |
| Cycle Description | (mg/mL) | (%) | (%)^{a} | % LMMS | Present |
| Pre-lyo composition | 0.625 | N/A | 77 | 98.4 | N/A |
| | | | | 0.9 | |
| | | | | 0.7 | |
| Target^{b} | 0.486 | NMT 0.2 | 80 | 96.3 | No |
| | | | | 2.7 | |
| | | | | NMT 1.6 | |
| Cumulative Agressive | 0.4 | NMT 0.2 | 64 | 96.9^{c} | NT |
| (“High-Slow”) | | | | 2.3^{c} | |
| | | | | 0.8^{c} | |
| Freezing Ramp | 0.463 | 0.3 | 62 | 98.3 | No |
| Aggressive | | | | 1.3 | |
| | | | | NMT 0.4 | |
| Annealing Temp | 0.465 | 0.3^{d} | 70 | 97.4 | No |
| Aggressive | | | | 2.0 | |
| | | | | 0.5 | |
| Annealing Ramp | 0.465 | 0.3 | 81 | 96.7 | NT |
| Aggressive | | | | 2.7 | |
| | | | | 0.6 | |
| Drying | 0.469 | 0.3 | 67 | 97.9 | NT |
| Temp + Ramp Aggressive | | | | 1.7 | |
| | | | | NMT 0.4 | |

^{a}Prefusion Content values determined by AM14 Fab titration by SEC as disclosed in Example 1
^{b}T0
^{c}Average of n = 2 results
^{d}Reported highest value of 2 samples
Abbreviations:
MHH = mannitol hemihydrate;
N/A = not applicable;
NT = Not Tested;
HMMS = high molecular weight species;
LMMS = low molecular weight species;
NMT = not more than

In a further experiment, the composition of Table 17 was lyophilized using the cycles described in Table 18 to assess robustness of the target lyophilization cycle. The cycles assessed the lyophilization parameters of freezing ramp rate, annealing ramp rate and temperature, and drying ramp rate Based on development experience from the drying optimization cycles and the critical process parameter assessment cycles along with historical development experience with lyophilization of semi-crystalline formulations, three cycles were created that expanded the target value parameters for temperature, ramp rate and chamber pressure. A “Low-Fast” (low temperature, low chamber pressure, fast ramp rate) cycle and two “High-Slow” cycles (high temperature, high chamber pressure, slow ramp rate) were executed using the parameters listed in Table 18. The two “High-Slow” cycles differ only by freezing ramp rate (0.25° C./min and 0.4° C./min) and were created as a result of the critical process parameter assessment that identified freezing ramp rate as a critical parameter impacting prefusion content in the preceding experiment.

TABLE 17

| | Pre-lyo | Post-Lyo Quantity | Composition Post- |
|---|---|---|---|
| | composition | per vial for 0.51 mL | Reconstitution with Sterile |
| Ingredient | (mg/mL or g/L) | Fill Volume (mg) | Water Diluent (mg/mL) |
| RSV A | 0.16 | 0.08 | 0.12 |
| RSV B | 0.16 | 0.08 | 0.12 |
| Tromethamine^{b} | 0.30^{d} | 0.15 | 0.23^{e} |
| Tris-HCl | 2.76^{d} | 1.41 | 2.07^{e} |
| Sucrose | 30 | 15 | 22.5 |
| Mannitol | 60 | 31 | 45.0 |
| Polysorbate 80 | 0.20 | 0.10 | 0.15 |
| Sodium Chloride | 2.92^{f} | 1.49 | 2.199 |
| Water for Injection | q.s to 1 mL or L | q.s to 0.51 mL | q.s to 1 mL |

^{b}Also known as Trometamol and Tris Base
^{c} Also known as Tris (hydroxymethyl) aminomethane hydrochloride (Tris-HCl), Tromethamine HCl, and Trometamol HCl
^{d}Tromethamine + Tris-Hydrochloride composition is equivalent to 20 mM Tris
^{e}Tromethamine + Tris-Hydrochloride composition is equivalent to 15 mM Tris
^{f}Equivalent to 50 mM NaCl
^{g}Equivalent to 37.5 mM NaCl

TABLE 18
Lyophilization Process Robustness Study Cycles

| | | High- | High- | Low- |
|---|---|---|---|---|
| Parameter | Target | Slow 1 | Slow 2 | Fast |
| Freezing Temp (° C.) | −50 | −45 | −45 | −55 |
| Freezing Ramp Rate (° C./min) | 1 | 0.25 | 0.4 | 1.5 |
| Annealing Temp (° C.) | −10 | −5 | −5 | −15 |
| Annealing Ramp Rate (° C./min) | 0.5 | 0.2 | 0.2 | 1.5 |
| Annealing Hold Time (h) | 4.5 | 4.5 | 4.5 | 4.5 |
| Re-freezing Temp (° C.) | −50 | −45 | −45 | −55 |
| Re-freezing Ramp Rate (° C./min) | 0.5 | 0.5 | 0.5 | 0.5 |
| Re-freezing Hold (h) | 1 | 1 | 1 | 1 |
| First Dry Ramp Rate (° C./min) | 0.5 | 0.1 | 0.1 | 1.0 |
| Drying Chamber Pressure | 200/267 | 250/334 | 250/334 | 150/200 |
| (mTorr/μbar) | | | | |
| First Dry Hold Time (h) | N/A | N/A | N/A | N/A |
| First Dry Hold Temp (° C.) | 0 | +5 | +5 | −5 |
| Second Dry Ramp Rate (° C./min) | 0.2 | 0.1 | 0.1 | 1.0 |
| Second Dry Temp (° C.) | 50 | 55 | 55 | 45 |
| Second Dry Temp Hold Time (h) | 8.6 | 8.6 | 8.6 | 8.6 |
| Drying Chamber Pressure | 200/267 | 250/334 | 250/334 | 150/200 |
| (mTorr/μbar) | | | | |

Prefusion content as measured by AM14 Fab titration by SEC is presented in Table 19 for all cycles for up to 8 months at 2-8° C., 25° C., and 40° C. The results from SEC-Fab Titration for % Prefusion Content indicate that a drop in prefusion content is observed upon lyophilization, as historically observed, but the amount of loss of prefusion content for High-Slow 2, Low-Fast and Target is similar for each cycle within method variability. All cycles demonstrated minimal loss in prefusion content from T0 to T8M timepoints across all conditions, except for the High-Slow 1 cycle at 40° C. It is noted that significant variability is observed for all cycles at the 1 month and 3 months timepoint. In general, all timepoints at each condition for the High-Slow 1 cycle demonstrate a lower prefusion content after 1 month when compared to the other cycles with some values falling outside of the acceptance criteria. As this cycle demonstrated the slowest freezing rate, it correlates to the previous findings in the critical parameter assessment development that the freezing rate of 0.25° C./min results in a greater loss in prefusion content when compared to the target cycle.

TABLE 19
Prefusion Content by AM14 Fab titration by SEC

| Cycle ID | Timepoint | Temperature | | Prefusion Content (%)^{a} |
|---|---|---|---|---|
| Pre-lyo Prep 1 | T0 | N/A | | 85 |
| | | | | 84 |
| Pre-lyo Prep 2 | T0 | N/A | | 81 |
| | | | | 82 |
| High-Slow 1 | T0 | N/A | | 64 |
| | | | | 68 |
| | 1 M | 2-8° | C. | 52 |
| | | | | 44 |
| | 3 M | | | 49 |
| | 8 M | | | 69 |
| | 1 M | 25° | C. | 51 |
| | | | | 65 |
| | 3 M | | | 53 |
| | 8 M | | | 63 |
| | 1 M | 40° | C. | 51 |
| | | | | 46 |
| | 3 M | | | 52 |
| | 8 M | | | 53 |
| High-Slow 2 | T0 | N/A | | 61 |
| | | | | 73 |
| | 1 M | 2-8° | C. | 68 |
| | | | | 65 |
| | 3 M | | | 62 |
| | 8 M | | | 72 |
| | 1 M | 25° | C. | 72 |
| | | | | 59 |
| | 3 M | | | 74 |
| | 8 M | | | 71 |
| | 1 M | 40° | C. | 52 |
| | | | | 68 |
| | 3 M | | | 59 |
| | 8 M | | | 61 |
| Low-Fast | T0 | N/A | | 76 |
| | | | | 82 |
| | 1 M | 2-8° | C. | 53 |
| | | | | 53 |
| | 3 M | | | 56 |
| | | | | 59 |
| | 8 M | | | 73 |
| | 1 M | 25° | C. | 63 |
| | | | | 71 |
| | 3 M | | | 66 |
| | | | | 68 |
| | 8 M | | | 76 |
| | 1 M | 40° | C. | 65 |
| | | | | 70 |
| | 3 M | | | 70 |
| | | | | 66 |
| | 8 M | | | 63 |
| Target | T0 | N/A | | 68 |
| | | | | 77 |
| | 1 M | 2-8° | C. | 59 |
| | | | | 69 |
| | 3 M | | | 69 |
| | | | | 70 |
| | 8 M | | | 73 |
| | 1 M | 25° | C. | 65 |
| | | | | 65 |
| | 3 M | | | 71 |
| | 8 M | | | 75 |
| | 1 M | 40° | C. | 64 |
| | | | | 66 |
| | 3 M | | | 72 |
| | 8 M | | | 67 |

^{a}Reported as an average of 2 replicates
Abbreviations:
M = month;
N/A = not applicable

### Size Exclusion Chromatography of Liquid RSV Composition (Pre-Lyo) and Lyo RSV Composition (Reconstituted)

SE HPLC results are presented in Table 20 for all cycles for up to 8 months at 2-8° C., 25° C., and 40° C. The results from SE-HPLC indicate minimal increase in HMMS for the samples produced by the high-slow 2, low-fast and target cycles from T0 to T8M (8 months) timepoints at 2-8° C. and 25° C. All timepoints for each condition for the High-Slow 1 cycle demonstrated a higher HMMS content along with a higher rate of increase in HMMS at 25° C. and 40° C. when compared to the other cycles. As this cycle demonstrated the slowest freezing rate, it correlates to the previous development history that a slower freezing rate can result in an increase in HMMS.

TABLE 20
Trimer, LMMS, and HMMS by SE-HPLC of Lyo composition

| | Time | | | HMMS | LMMS | Trimer |
|---|---|---|---|---|---|---|
| Cycle ID | point | Temp. | | (%) | (%) | (%) |
| Pre-lyo Prep 1 | T0 | N/A | | 2.4 | 0.9 | 96.7 |
| Pre-lyo Prep 2 | T0 | N/A | | 2.8 | 1.0 | 96.2 |
| High-Slow 1 | T0 | N/A | | 3.2 | 1.1 | 95.6 |
| | 1 M | 2-8° | C. | 4.7 | 1.9 | 93.5 |
| | | | | 4.0 | 1.5 | 94.5 |
| | 3 M | | | 4.3 | NMT 0.8 | 95 |
| | 8 M | | | 3.5 | 1.3 | 95.1 |
| | 1 M | 25° | C. | 6.3 | 1.8 | 91.9 |
| | | | | 5.5 | 1.9 | 92.6 |
| | 3 M | | | 4.4 | NMT 0.8 | 94.9 |
| | 8 M | | | 4.8 | 1.5 | 93.6 |
| | 1 M | 40° | C. | 4.6 | 1.1 | 94.3 |
| | | | | 4.0 | 1.0 | 95 |
| | 3 M | | | 5.9 | NMT 0.8 | 93.3 |
| | 8 M | | | 19.3 | 2.1 | 78.5 |
| High-Slow 2 | T0 | N/A | | 2.6 | 1.1 | 96.3 |
| | 1 M | 2-8° | C. | 3.4 | 1.0 | 95.5 |
| | | | | 2.9 | 1.0 | 96.1 |
| | 3 M | | | 4.4 | NMT 0.8 | 94.9 |
| | 8 M | | | 3.1 | 1.2 | 95.7 |
| | 1 M | 25° | C. | 3.5 | 1.0 | 95.5 |
| | | | | 3.3 | 0.9 | 95.8 |
| | 3 M | | | 4.2 | NMT 0.8 | 95.2 |
| | 8 M | | | 3.3 | 1.3 | 95.4 |
| | 1 M | 40° | C. | 3.1 | 1.7 | 95.2 |
| | | | | 2.8 | 1.6 | 95.6 |
| | 3 M | | | 4.6 | NMT 0.8 | 94.7 |
| | 8 M | | | 13.2 | 1.7 | 85.1 |
| Low-Fast | T0 | N/A | | 2.5 | 1.0 | 96.5 |
| | 1 M | 2-8° | C. | 2.2 | 1.3 | 96.5 |
| | | | | 2.2 | 1.4 | 96.4 |
| | 3 M | | | 3.1 | NMT 0.8 | 96.2 |
| | 8 M | | | 2.9 | 1.4 | 95.7 |
| | 1 M | 25° | C. | 2.3 | 1.2 | 96.5 |
| | | | | 2.3 | 1.4 | 96.3 |
| | 3 M | | | 3.2 | NMT 0.8 | 96.2 |
| | 8 M | | | 3.2 | 1.3 | 95.5 |
| | 1 M | 40° | C. | 2.3 | 1.3 | 96.4 |
| | | | | 2.6 | 1.3 | 96.1 |
| | 3 M | | | 4.6 | NMT 0.8 | 94.8 |
| | 8 M | | | 20.9 | 2.2 | 77 |
| Target | T0^{a} | N/A | | 2.1 | 1.0 | 96.9 |
| | 1 M | 2-8° | C. | 2.5 | 1.4 | 96.2 |
| | | | | 2.4 | 1.1 | 96.5 |
| | 3 M | | | 4.0 | NMT 0.8 | 95.4 |
| | 8 M | | | 3.0 | 1.3 | 95.8 |
| | 1 M | 25° | C. | 3.2 | 1.0 | 95.7 |
| | | | | 2.9 | 1.1 | 96 |
| | 3 M | | | 3.3 | NMT 0.8 | 96.3 |
| | 8 M | | | 3.5 | 1.3 | 95.3 |
| | 1 M | 40° | C. | 2.9 | 1.7 | 95.4 |
| | | | | NT | NT | NT |
| | | | | 3.9 | NMT 0.8 | 95.4 |
| | 8 M | | | 9.8 | 1.8 | 88.5 |

Abbreviations:
M = month;
N/A = not applicable;
NMT = not more than;
HMMS = high molecular mass species;
LMMS = low molecular mass species

The data of Tables 19 and 20 confirmed that the freezing ramp rate is a critical parameter of a method for lyophilizing an aqueous immunogenic composition comprising an RSV F protein trimer in the profusion conformation. In particular, it was observed that a freezing ramp rate of 0.2° C./min results in a substantial loss of prefusion content.

### LISTING OF RAW SEQUENCES

SEQ ID NO: 1: amino acid sequence of a construct of F1 polypeptide of RSV F protein of
subtype A:
FLGFLLGVGSACASGIAVSKVLHLEGEVNKIKSALLSTNKAVVSLSNGVSVLTIKVLDLKNYIDKQ
LLPIVNKQSCSISNIETVIEFQQKNNRLLEITREFSVNAGVTTPVSTYMLTNSELLSLINDMPITND
QKKLMSSNVQIVRQQSYSIMSIIKEEVLAYVVQLPLYGVIDTPCWKLHTSPLCTTNTKEGSNICL
TRTDRGWYCDNAGSVSFFPQAETCKVQSNRVFCDTMNSLTLPSEVNLCNIDIFNPKYDCKIMT
SKTDVSSSVITSLGAIVSCYGKTKCTASNKNRGIIKTFSNGCDYVSNKGVDTVSVGNTLYYVNK
QEGKSLYVKGEPIINFYDPLVFPSSEFDASISQVNEKINQSLAFIRKSDELLSAIGGYIPEAPRDG
QAYVRKDGEWVLLSTFL
SEQ ID NO: 2: amino acid sequence of a construct of F2 polypeptide of RSV F protein of
subtype A:
QNITEEFYQSTCSAVSKGYLSALRTGWYTSVITIELSNIKENKCNGTDAKVKLIKQELDKYKNAV
TELQLLMQSTPACNSRARR
SEQ ID NO: 3: amino acid sequence of a construct of F1 polypeptide of RSV F protein of
subtype B
FLGFLLGVGSACASGIAVSKVLHLEGEVNKIKNALLSTNKAVVSLSNGVSVLTIKVLDLKNYINNQ
LLPIVNQQSCRISNIETVIEFQQKNSRLLEITREFSVNAGVTTPLSTYMLTNSELLSLINDMPITND
QKKLMSSNVQIVRQQSYSIMSIIKEEVLAYVVQLPIYGVIDTPCWKLHTSPLCTTNIKEGSNICLT
RTDRGWYCDNAGSVSFFPQADTCKVQSNRVFCDTMNSLTLPSEVSLCNTDIFNSKYDCKIMT
SKTDISSSVITSLGAIVSCYGKTKCTASNKNRGIIKTFSNGCDYVSNKGVDTVSVGNTLYYVNKL
EGKNLYVKGEPIINYYDPLVFPSSEFDASISQVNEKINQSLAFIRRSDELLSAIGGYIPEAPRDGQ
AYVRKDGEWVLLSTFL
SEQ ID NO: 4: amino acid sequence a construct of F2 polypeptide of RSV F protein of
subtype B
QNITEEFYQSTCSAVSRGYFSALRTGWYTSVITIELSNIKETKCNGTDTKVKLIKQELDKYKNAV
TELQLLMQNTPACNNRARR
SEQ ID NO: 5. Amino Acid Sequence of the Full Length F0 of Native RSV A2 (GenBank GI:
138251; Swiss Prot P03420)
MELLILKANAITTILTAVTFCFASGQNITEEFYQSTCSAVSKGYLSALRTGWYTSVITIELSNIKEN
KCNGTDAKVKLIKQELDKYKNAVTELQLLMQSTPPTNNRARRELPRFMNYTLNNAKKTNVTLS
KKRKRRFLGFLLGVGSAIASGVAVSKVLHLEGEVNKIKSALLSTNKAVVSLSNGVSVLTSKVLDL
KNYIDKQLLPIVNKQSCSISNIETVIEFQQKNNRLLEITREFSVNAGVTTPVSTYMLTNSELLSLIN
DMPITNDQKKLMSNNVQIVRQQSYSIMSIIKEEVLAYVVQLPLYGVIDTPCWKLHTSPLCTTNTK
EGSNICLTRTDRGWYCDNAGSVSFFPQAETCKVQSNRVFCDTMNSLTLPSEINLCNVDIFNPK
YDCKIMTSKTDVSSSVITSLGAIVSCYGKTKCTASNKNRGIIKTFSNGCDYVSNKGMDTVSVGN
TLYYVNKQEGKSLYVKGEPIINFYDPLVFPSDEFDASISQVNEKINQSLAFIRKSDELLHNVNAG
KSTTNIMITTIIIVIIVILLSLIAVGLLLYCKARSTPVTLSKDQLSGINNIAFSN
SEQ ID NO: 6. Amino Acid Sequence of the Full Length F0 of Native RSV B (18537 strain;
GenBank GI: 138250; Swiss Prot P13843)
MELLIHRSSAIFLTLAVNALYLTSSQNITEEFYQSTCSAVSRGYFSALRTGWYTSVITIELSNIKET
KCNGTDTKVKLIKQELDKYKNAVTELQLLMQNTPAANNRARREAPQYMNYTINTTKNLNVSISK
KRKRRFLGFLLGVGSAIASGIAVSKVLHLEGEVNKIKNALLSTNKAVVSLSNGVSVLTSKVLDLK
NYINNRLLPIVNQQSCRISNIETVIEFQQMNSRLLEITREFSVNAGVTTPLSTYMLTNSELLSLIND
MPITNDQKKLMSSNVQIVRQQSYSIMSIIKEEVLAYVVQLPIYGVIDTPCWKLHTSPLCTTNIKEG
SNICLTRTDRGWYCDNAGSVSFFPQADTCKVQSNRVFCDTMNSLTLPSEVSLCNTDIFNSKYD
CKIMTSKTDISSSVITSLGAIVSCYGKTKCTASNKNRGIIKTFSNGCDYVSNKGVDTVSVGNTLY
YVNKLEGKNLYVKGEPIINYYDPLVFPSDEFDASISQVNEKINQSLAFIRRSDELLHNVNTGKST
TNIMITTIIIVIIVVLLSLIAIGLLLYCKAKNTPVTLSKDQLSGINNIAFSK
SEQ ID NO: 7: Amino acid Sequence of the T4 Fibritin Foldon:
GYIPEAPRDGQAYVRKDGEWVLLSTFL
SEQ ID NO: 8: Amino Acid Sequence of Heavy Chain Variable Domain of Antibody D25:
QVQLVQSGAEVKKPGSSVMVSCQASGGPLRNYIINWLRQAPGQGPEWMGGIIPVLGTVHYAP
KFQGRVTITADESTDTAYIHLISLRSEDTAMYYCATETALVVSTTYLPHYFDNWGQGTLVTVSS
SEQ ID NO: 9: Amino Acid Sequence of Light Chain Variable Domain of Antibody D25:
DIQMTQSPSSLSAAVGDRVTITCQASQDIVNYLNWYQQKPGKAPKLLIYVASNLETGVPSRFSG
SGSGTDFSLTISSLQPEDVATYYCQQYDNLPLTFGGGTKVEIKR
SEQ ID NO: 10: Amino Acid Sequence of Heavy Chain Variable Domain of Antibody AM14:
EVQLVESGGGVVQPGRSLRLSCAASGFSFSHYAMHWVRQAPGKGLEWVAVISYDGENTYYA
DSVKGRFSISRDNSKNTVSLQMNSLRPEDTALYYCARDRIVDDYYYYGMDVWGQGATVTVSS
SEQ ID NO: 11: Amino Acid Sequence of Light Chain Variable Domain of Antibody AM14:
DIQMTQSPSSLSASVGDRVTITCQASQDIKKYLNWYHQKPGKVPELLMHDASNLETGVPSRFS
GRGSGTDFTLTISSLQPEDIGTYYCQQYDNLPPLTFGGGTKVEIKRTV
SEQ ID NO: 12: Amino Acid Sequence of Heavy Chain Variable Domain of Antibody AM22
QVQLVQSGAEVKKPGATVKVSCKISGHTLIKLSIHWVRQAPGKGLEWMGGYEGEVDEIFYAQK
FQHRLTVIADTATDTVYMELGRLTSDDTAVYFCGTLGVTVTEAGLGIDDYWGQGTLVTVSS
SEQ ID NO: 13: Amino Acid Sequence of Light Chain Variable Domain of Antibody AM22
EIVLTQSPGTLSLSPGERATLSCRASQIVSRNHLAWYQQKPGQAPRLLIFGASSRATGIPVRFS
GSGSGTDFTLTINGLAPEDFAVYYCLSSDSSIFTFGPGTKVDFK

1. An aqueous immunogenic composition comprising
(i) a first RSV F protein trimer in the prefusion conformation;
(ii) sodium chloride at a concentration of between about 20 mM and about 100 mM;
(iii) glycine at a concentration of between about 10 mg/mL and about 100 mg/mL or sucrose is at a concentration of between about 10 mg/mL and about 70 mg/mL and mannitol at a concentration of between about 10 mg/mL and about 70 mg/mL; and
(iv) a buffer;
wherein the pH of said composition is between about 7 and about 8.

2.-6. (canceled)

7. The aqueous immunogenic composition according to claim 1, wherein the ratio of sucrose to mannitol is between 1 to 1 and 1 to 5.

8. The aqueous immunogenic composition according to claim 1, wherein the composition further comprises a surfactant.

9. The aqueous immunogenic composition according to claim 8, wherein the surfactant is polysorbate 80.

10. The aqueous immunogenic composition according to claim 1, wherein the buffer is Tris (tris(hydroxymethyl)aminomethane).

11. The aqueous immunogenic composition according to claim 1, wherein the composition comprises
(i) a first RSV F protein trimer in the prefusion conformation;
(ii) sodium chloride at a concentration of between about 35 mM and about 65 mM;
(iii) sucrose at a concentration of between about 20 mg/mL and about 40 mg/mL and mannitol at a concentration of between about 45 mg/mL and about 75 mg/mL;
(iv) a Tris buffer at a concentration of between about 15 mM and about 25 mM;
(v) polysorbate 80 at a concentration of between about 0.1 and about 0.3 mg/mL;
wherein the pH of the composition is between 7.1 and 7.7.

12. The aqueous immunogenic composition according to claim 1, wherein the composition comprises
(i) a first RSV F protein trimer in the prefusion conformation;
(ii) sodium chloride at a concentration of about 50 mM;
(iii) sucrose at a concentration of about 30 mg/mL and mannitol at a concentration of about 60 mg/mL;
(iv) a Tris buffer at a concentration of about 20 mM;
(v) polysorbate 80 at a concentration of about 0.2 mg/mL;
wherein the pH of the composition is about 7.4.

13. The aqueous immunogenic composition according to claim 1, wherein the first RSV F protein is a F protein of subtype A.

14. The aqueous immunogenic composition according to claim 13, wherein the first RSV F protein comprises a combination of mutations relative to the corresponding wild-type RSV F protein, wherein the combination of mutations is selected from the group consisting of:
(1) combination of 103C, 148C, 190I, and 486S;
(2) combination of 54H, 55C, 188C, and 486S;
(3) combination of 54H, 103C, 148C, 190I, 296I, and 486S;
(4) combination of 54H, 55C, 142C, 188C, 296I, and 371C;
(5) combination of 55C, 188C, and 486S;
(6) combination of 54H, 55C, 188C, and 190I;
(7) combination of 55C, 188C, 190I, and 486S;
(8) combination of 54H, 55C, 188C, 190I, and 486S;
(9) combination of 155C, 190I, 290C, and 486S;
(10) combination of 54H, 55C, 142C, 188C, 296I, 371C, 486S, 487Q, and 489S;
(11) combination of 54H, 155C, 190I, 290C, and 296I, and
(12) combination of 155C, 190F, 290C, and 207L.

15. The aqueous immunogenic composition according to claim 13, wherein the first RSV F protein comprises a combination of mutations relative to the corresponding wild-type RSV F protein, wherein the combination of mutations is selected from the group consisting of:
(1) combination of 215P and 486N,
(2) combination of 66E, 215P, and 486N,
(3) combination of 66E, 76V, 215P, and 486N, and,
(4) combination of 66E, 671, 76V, 215P, and 486N.

16. The aqueous immunogenic composition according to claim 1, wherein the first RSV F protein comprises a trimerization domain.

17. The aqueous immunogenic composition according to claim 1, wherein the composition further comprises a second RSV F protein trimer in the prefusion conformation.

18. The aqueous immunogenic composition according to claim 17, wherein the second RSV F protein is a F protein of subtype B.

19. The aqueous immunogenic composition according to claim 18, wherein the second RSV F protein comprises a combination of mutations relative to the corresponding wild-type RSV F protein, wherein the combination of mutations is selected from the group consisting of:
(1) combination of 103C, 148C, 190I, and 486S;
(2) combination of 54H, 55C, 188C, 486S;
(3) combination of 54H, 103C, 148C, 190I, 296I, and 486S;
(4) combination of 54H, 55C, 142C, 188C, 296I, and 371C;
(5) combination of 55C, 188C, and 486S;
(6) combination of 54H, 55C, 188C, and 190I;
(7) combination of 55C, 188C, 190I, and 486S;
(8) combination of 54H, 55C, 188C, 190I, and 486S;
(9) combination of 155C, 190I, 290C, and 486S;
(10) combination of 54H, 55C, 142C, 188C, 296I, 371C, 486S, 487Q, and 489S;
(11) combination of 54H, 155C, 190I, 290C, and 296I, and
(12) combination of 155C, 190F, 290C, and 207L.

20. The aqueous immunogenic composition according to claim 17, wherein the second RSV F protein comprises a combination of mutations relative to the corresponding wild-type RSV F protein, wherein the combination of mutations is selected from the group consisting of:
(1) combination of 215P and 486N,
(2) combination of 66E, 215P, and 486N,
(3) combination of 66E, 76V, 215P, and 486N, and,
(4) combination of 66E, 671, 76V, 215P, and 486N.

21. The aqueous immunogenic composition according to claim 17, wherein the second RSV F protein comprises a trimerization domain.

22. A method for storing an aqueous immunogenic composition according to claim 1 comprising an RSV F protein trimer in the prefusion conformation comprising storing the composition at a temperature of between about 15° C. and about 30° C.

23. (canceled)

24. The method according to claim 22, wherein the RSV F protein is a F protein of subtype A or subtype B or both subtype A and subtype B.

25. (canceled)

26. (canceled)

27. A method for lyophilizing an aqueous immunogenic composition according to claim 1 comprising the step of freezing the composition wherein said step comprises reducing the temperature to a freezing temperature comprised between −40° C. and −60° C. and at a freezing ramp rate of at least about 0.3° C./min.

28. (canceled)

29. A lyophilized immunogenic composition obtained by the method according to claim 27.

30. (canceled)

31. A kit comprising:
(i) a lyophilized composition, and
(ii) a diluent for reconstituting the lyophilized composition;
wherein the reconstitution of the lyophilized composition with the diluent results in an aqueous immunogenic composition according to claim 1.

32.-37. (canceled)

38. The aqueous immunogenic composition according to claim 2, wherein the concentration ratio of sucrose to mannitol is between 1 to 2 and 1 to 4.
